(19) United States
(12) Patent Application Publication
(10) Pub. No.: US 2024/0142457 A1
(43) Pub. Date: May 2, 2024

(54) USE OF CASD1 AS A BIOMARKER OF A CANCER EXPRESSING THE O-ACETYLATED-GD2 GANGLIOSIDE

(71) Applicants: OGD2 PHARMA, Nantes (FR); UNIVERSITE DE LILLE, Lille (FR); CENTRE NATIONAL DE LA RECHERCHE SCIENTIFIQUE, Paris (FR)

(72) Inventors: Sophie GROUX-DEGROOTE, Villeneuve d'Ascq (FR); Philippe DELANNOY, Villeneuve d'Ascq (FR); Sumeyye CAVDARLI, Nantes (FR); Jean-Marc LE DOUSSAL, Nantes (FR); Angélina KASPROWICZ, Nantes (FR)

(73) Assignees: OGD2 PHARMA, Nantes (FR); UNIVERSITE DE LILLE, Lille (FR); CENTRE NATIONAL DE LA RECHERCHE SCIENTIFIQUE, Paris (FR)

(21) Appl. No.: 18/568,521

(22) PCT Filed: Jun. 10, 2022
(86) PCT No.: PCT/EP2022/065896

§ 371 Date: Dec. 8, 2023

(30) Foreign Application Priority Data
Jun. 10, 2021 (EP) 21305797.9

(51) Int. Cl. G01N 33/574; C12Q 1/6886

(52) U.S. Cl. CPC G01N 33/57484; C12Q 1/6886; C12Q 2600/158; G01N 2333/91051

(57) ABSTRACT

The use of CASD1 as a biomarker of a cancer expressing the O-acetylated-GD2 ganglioside. Also, a method of diagnosing a cancer expressing the O-acetylated-GD2 ganglioside, a method of selecting a subject suffering from a cancer expressing the O-acetylated-GD2 ganglioside for treatment targeting the cancer, or a method of monitoring the response of a subject suffering from a cancer expressing the O-acetylated-GD2 ganglioside to a treatment targeting the cancer.

### FIELD OF INVENTION

The present invention relates to the use of CASD1 as a biomarker of a cancer expressing the O-acetylated-GD2 ganglioside. The present invention further concerns a method of diagnosing a cancer expressing the O-acetylated-GD2 ganglioside, a method of selecting a subject suffering from a cancer expressing the O-acetylated-GD2 ganglioside for treatment targeting said cancer, or a method of monitoring the response of a subject suffering from a cancer expressing the O-acetylated-GD2 ganglioside to a treatment targeting said cancer.

### BACKGROUND OF INVENTION

Changes in cell surface glycosylation that affect both membrane glycolipids and glycoproteins occur during malignant transformation. Different cancer-associated glycans have been so far characterized as tumor associated carbohydrate antigens (TACA) and are involved in the exacerbation of tumor aggressiveness. In that light, complex gangliosides such as GD2 and GD3 have been characterized as oncofetal markers of melanoma and neuroblastoma. Besides, GD2 is also highly expressed in breast cancer patients with aggressive cancer subtypes. The biosynthesis of GD3 and GD2 is controlled by two glycosyltransferases, GD3 synthase (ST8SIA I, GD3S) and GD2 synthase (B4GALNT1, GD2S), respectively. Basically, gangliosides are acidic glycosphingolipids carrying one or more sialic acid residues in their carbohydrate moiety and are mainly located in lipid rafts at the outer leaflet of the plasma membrane. They are found in different cell types as a mixture of di-, tri-, tetra- saccharide structures, which confers to gangliosides a high structural heterogeneity. Complex gangliosides from b- and c-series with two or more sialic acid residues linked to lactosyl-ceramide are usually absent from normal adult tissues except the nervous system but are re-expressed in tumors from neuro-ectoderm origin where they exhibit a pro-tumoral action, enhancing tumor aggressiveness mainly through cis- and trans- interactions with tyrosine kinase receptors and the microenvironment. The inventors have previously shown that GD2 interacts with c-Met tyrosine kinase receptor in MDA-MB-231 breast cancer cells and induces the activation of PI3K/Akt and MEK/ERK signaling pathways. Considering its expression and pro-tumoral activity in tumors from neuro-ectoderm origin, GD2 was extensively studied as target antigen for immunotherapy. In 2015, Dinutuximab (Unituxin™) monoclonal antibody (mAb) has been approved by the Food Drug Administration for the treatment of pediatric high-risk neuroblastoma. However, the anti-GD2 mAb treatment caused severe side effects due to the expression of GD2 in healthy peripheral nerve fibers

It was previously demonstrated that a modified form of GD2—the O-acetylated GD2 ganglioside (OAcGD2)—has, in contrast to GD2, a safer expression pattern, with no or very limited expression on normal tissues and typically absence of OAcGD2 expression in the peripheral nerve fibers, pituitary gland or human brain cells, OAcGD2 being exclusively expressed in cancer tissues. A mouse therapeutic antibody (8B6) targeting the OAcGD2 was generated, as well as a human-mouse chimeric antibody, named c8B6. It was shown that the anti-OAcGD2 c8B6 mAb induced in vitro mitochondrial cell death and cell cycle arrest in a mouse model of neuroblastoma, and decreased tumor growth without inducing allodynia in vivo. Moreover, the administration of the mouse therapeutic antibody 8B6 targeting the OAcGD2 is not associated with any neurotoxicity, especially due to the absence of expression of this cancer antigen on healthy cells, notably on peripheral nerve fibers. The human-mouse chimeric antibody c8B6 shows no cross-reaction, neither with GD2, nor with others gangliosides and shows the absence of OAcGD2 antigen expression in the normal brain tissue. In animal models, anti-OAcGD2 chimeric antibodies display similar anti-tumor activity than anti-GD2 monoclonal antibodies (mAbs), while avoiding their toxicity, indicating that OAcGD2 is a better tumor-associated antigen than GD2 and that anti-OAcGD2 mAbs are best-in-class antibodies capable to reduce the uncomfortable side effects commonly associated with anti-GD2 mAb therapies and improve quality of life of patients.

The O-acetylated GD2 ganglioside is expressed in cancerous tissues of various cancer types. Anti-OAcGD2 mAbs could be highly benefic for patients suffering from cancers expressing the O-acetylated-GD2 ganglioside. However, the quantification of complex gangliosides, such as GD2 and its the O-acetylated form (OAcGD2), in general as well as in patient cells or tissues, remains challenging.

Therefore, there is still a need for new method enabling the identification of patients suffering from cancers expressing the O-acetylated-GD2 ganglioside, who could benefit from an anti-cancer treatment comprising anti-OAcGD2 mAbs. In this context, the identification of new biomarkers of cancers expressing the O-acetylated-GD2 ganglioside would be highly useful.

The biosynthesis of GD2 is very well described, but the mechanism of its O-acetylation remains currently unclear.

Ganglioside biosynthesis occurs in a stepwise manner by the sequential addition of glucose, galactose, N-acetylgalactosamine and sialic acid residues on the ceramide moiety. GD2 is synthesized by the transfer of one N-acetylgalactosamine residue onto GD3. The inventors previously analyzed the expression of O-acetylated and non-O-acetylated gangliosides in different cancer cell lines and identified OAcGD2 expression in breast cancer, melanoma and neuroblastoma cells. MALDI-MS analysis showed that O-acetylation occurred either on the sub-terminal or the terminal sialic acid residue of the carbohydrate moiety. Sialic acids are a family of 9-carbon monosaccharides derived from neuraminic acid (Neu5Ac) that can be acetylated on the OH group of carbon-4, -7, -8 or -9. The inventors have determined the precise position of O-acetyl substitution on sialic acid residue in breast cancer gangliosides and shown that gangliosides expressed by breast cancer cells are mainly acetylated on the carbon 9, forming Neu5,9Ac₂, suggesting that breast cancer cells mainly express 9-OAcGD2. Two OAcGD2 isomers were identified by MS/MS fragmentation in breast cancer cells, with the O-acetyl group either on the terminal or internal sialic acid residue.

O-acetylation of gangliosides takes place in the Golgi apparatus in a cell- and development-dependent manner Different levels of regulation including substrates availability, Golgi-ER transporter, and the balance between sialyl-O-acetyltransferase (SOAT) and sialyl-O-acetylesterase (SIAE) activities, control this process. All the attempts made for the biochemical isolation of mammalian SOATs were unsuccessful and over decades, the genetic basis of mammalian SOATs remained elusive. Recently, CASD1 (Cas1 domain containing 1) was identified as a putative human SOAT. CASD1 shares sequence similarity with Cas1 (capsule synthesis 1) of the fungal pathogen Cryptococcus neoformans, which catalyzes the transfer of O-acetyl groups at the C6 position of mannose residues of the cryptococcal capsular polysaccharide glucuronoxylomannan.

The inventors investigated the role of CASD1 in GD2 O-acetylation in engineered CHO and SUM159PT breast cancer cell lines. CASD1 expression was modulated in SUM159PT cells using plasmid transfection for overexpression and siRNA strategies for gene silencing. The inventors showed that OAcGD2 expression was reduced in SUM159PT transiently depleted for CASD1 expression. In parallel, OAcGD2 expression was increased in SUM159PT cells transiently overexpressing CASD1. The role of CASD1 in OAcGD2 synthesis was dissected in CHO cells. Co-expression of GD3S and GD2S induced the formation of 9-O-acetylated GD2 in CHO wild type but not in CHOΔCasd1 cells.

Thus, the inventors surprisingly showed that CASD1 is essential for the biosynthesis of the O-acetylated-GD2 ganglioside. Moreover, the inventors surprisingly showed that high expression level of CASD1 correlated with poor survival in many cancer types.

Therefore, CASD1 may be used as a biomarker of the expression of the O-acetylated-GD2 ganglioside, as well as a biomarker of cancers expressing the O-acetylated-GD2 ganglioside, notably as a prognostic biomarker in these cancers.

### SUMMARY

A first aspect of the invention relates to an in vitro method of selecting a subject suffering from a cancer expressing the O-acetylated-GD2 ganglioside for treatment targeting said cancer, said method comprising the steps of:
a1) measuring CASD1 expression level as a biomarker in a biological sample of the subject;
a1′) optionally, measuring the expression level of at least one biomarker selected from the group consisting of OAcGD2, GD2, GD2S, GD3S, CERK, PIK3C2A, PDK3, MERTK and NME3, in a biological sample of the subject; and
b) based on the levels measured at step a1), and optionally at step a1′), selecting said subject to undergo treatment targeting said cancer expressing the O-acetylated-GD2 ganglioside.

A second aspect of the invention relates to an in vitro method of monitoring the response of a subject suffering from a cancer expressing the O-acetylated-GD2 ganglioside to a treatment targeting said cancer, said method comprising the steps of:
a1) measuring CASD1 expression level as a biomarker in a biological sample of the subject;
a1′) optionally, measuring the expression level of at least one biomarker selected from the group consisting of OAcGD2, GD2, GD2S, GD3S, CERK, PIK3C2A, PDK3, MERTK and NME3, in a biological sample of the subject; and
b) based on the levels measured at step a1), and optionally at step a1′), monitoring the response of said subject to said treatment.

A third aspect of the invention relates to an in vitro method of diagnosing a cancer expressing the O-acetylated-GD2 ganglioside in a subject, said method comprising the steps of:
a1) measuring CASD1 expression level as a biomarker in a biological sample of the subject;
a1′) optionally, measuring the expression level of at least one biomarker selected from the group consisting of OAcGD2, GD2, GD2S, GD3S, CERK, PIK3C2A, PDK3, MERTK and NME3, in a biological sample of the subject; and
b) based on the levels measured at step a1), and optionally at step a1′), diagnosing a cancer expressing the O-acetylated-GD2 ganglioside in said subject.

According to some embodiments, the in vitro methods of the invention further comprise a step a2) of comparing the expression level measured at step a1), and/or optionally at step a1′), with a threshold value.

According to some embodiments, the subject is selected to undergo a treatment targeting said cancer expressing the O-acetylated-GD2 ganglioside, or is diagnosed as suffering from a cancer expressing the O-acetylated-GD2 ganglioside, if the expression level measured at step a1), and/or optionally at step a1′), is higher than the threshold value.

According to some embodiments, the biological sample is selected from the group consisting of a blood sample, a serum sample, a plasma sample, a urine sample, a tissue sample from a biopsy and a cell sample from a biopsy.

According to some embodiments, the expression level measured at step a1), and/or optionally at step a1′), is measured at the DNA or RNA level, preferably by RT-PCR, RT-qPCR, Northern Blot, hybridization techniques, microarrays or sequencing.

According to some embodiments, the expression level measured at step a1), and/or optionally at step a1′), is measured at the protein level, preferably by FACS, immunohistochemistry, mass spectrometry, western blot associated with cell fractionation, enzyme-linked immunosorbent assay (ELISA), sandwich ELISA, fluorescent-linked immunosorbent assay (FLISA), enzyme immunoassay (EIA), radioimmunoassay (RIA) or image analysis.

According to some embodiments, the treatment comprises an antibody that binds to the O-acetylated-GD2 ganglioside.

Another aspect of the invention relates to the use of CASD1 as a biomarker of a cancer expressing the O-acetylated-GD2 ganglioside.

According to some embodiments, the cancer expressing the O-acetylated-GD2 ganglioside is characterized by the presence of cells expressing the O-acetylated-GD2 ganglioside at their cell surface in the subject.

According to some embodiments, the cancer expressing the O-acetylated-GD2 ganglioside is selected from the group consisting of neuroblastoma, glioma (including glioblastoma), retinoblastoma, Ewing's family of tumors, sarcoma (including rhabdomyosarcoma, osteosarcoma, leiomyosarcoma, liposarcoma, and fibrosarcoma), lung cancer (including small cell lung cancer), breast cancer, melanoma (including uveal melanoma), metastatic renal carcinoma, head and neck cancer, hematological cancers (including leukemia, Hodgkin lymphoma, non-Hodgkin lymphoma and myeloma), colorectal cancer, pancreatic cancer, prostate cancer, liver cancer, bladder cancer, gastric/stomach cancer, cervical cancer, endometrial cancer, neuroendocrine cancer, esophageal cancer, ovarian cancer, skin cancer, kidney cancer, soft tissue sarcoma, adrenal cancer, thymic cancer (including thymoma), testicular cancer and thyroid cancer.

Another aspect of the invention relates to an inhibitor of CASD1 for use in the treatment of a cancer expressing the O-acetylated-GD2 ganglioside.

Still another aspect of the invention pertains to an inhibitor of a biomarker selected from the group consisting of CERK, PIK3C2A, PDK3, MERTK, NME3 and EZH2, in combination with a therapy targeting the O-acetylated-GD2 ganglioside, for use in the treatment of a cancer expressing the O-acetylated-GD2 ganglioside.

### DEFINITIONS

The term “administering” means either directly administering a compound or composition, or administering a prodrug, derivative or analog which will form an equivalent amount of the active compound or substance within the body. Exemplary routes of administration include, but are not limited to, injection (such as subcutaneous, intramuscular, intradermal, intraperitoneal, and intravenous), oral, sublingual, rectal, transdermal, mucosal, intranasal, vaginal and inhalation routes.

The term “antigen” refers to a compound, composition, or substance that can stimulate the production of antibodies or a T cell response in an animal, including compositions that are injected or absorbed into an animal An antigen reacts with the products of specific humoral or cellular immunity, including those induced by heterologous immunogens. The term “antigen” includes all related antigenic epitopes. “Epitope” or “antigenic determinant” refers to a site on an antigen to which B and/or T cells respond. Epitopes can be formed both from contiguous amino acids or noncontiguous amino acids juxtaposed by tertiary folding of a protein. Epitopes formed from contiguous amino acids are typically retained on exposure to denaturing solvents whereas epitopes formed by tertiary folding are typically lost on treatment with denaturing solvents. An epitope typically includes at least 3, and more usually, at least 5, about 9, or about 8-10 amino acids in a unique spatial conformation. Methods of determining spatial conformation of epitopes include, for example, x-ray crystallography and 2-dimensional nuclear magnetic resonance.

The term “antigen-binding fragment” refers to a part or region of an antibody, which comprises fewer amino acid residues than the whole antibody. An “antigen-binding fragment” binds antigen and/or competes with the whole antibody from which it was derived for antigen binding. Antibody-binding fragments encompasses, without any limitation, single chain antibodies, Fv, dsFv, Fab, Fab′, Fab′-SH, F(ab)′2, scFv, Fd, VHH, defucosylated antibodies, diabodies, triabodies and tetrabodies.

The term “decrease” refers to reducing the quality, amount, or strength of something.

The term “isolated” or “non-naturally occurring” with reference to a biological component (such as a nucleic acid molecule, protein, organelle or cells), refers to a biological component altered or removed from the natural state. For example, a nucleic acid or a peptide naturally present in a living animal is not “isolated,” but the same nucleic acid or peptide partially or completely separated from the coexisting materials of its natural state is “isolated”. An isolated nucleic acid or peptide can exist in substantially purified form, or can exist in a non-native environment such as, for example, a host cell. Typically, a preparation of isolated nucleic acid or peptide contains the nucleic acid or peptide at least about 80% pure, at least about 85% pure, at least about 90% pure, at least about 95% pure, greater than 95% pure, greater than about 96% pure, greater than about 97% pure, greater than about 98% pure, or greater than about 99% pure. Nucleic acids and proteins that are “non-naturally occurring” or have been “isolated” include nucleic acids and proteins purified by standard purification methods. The term also embraces nucleic acids and proteins prepared by recombinant expression in a host cell as well as chemically synthesized nucleic acids. An “isolated polypeptide” is one that has been identified and separated and/or recovered from a component of its natural environment.

The term “mutation” refers to any difference in a nucleic acid or polypeptide sequence from a normal, consensus or “wild type” sequence. A mutant is any protein or nucleic acid sequence comprising a mutation. In addition, a cell or an organism with a mutation may also be referred to as a mutant. Some types of coding sequence mutations include point mutations (differences in individual nucleotides or amino acids); silent mutations (differences in nucleotides that do not result in an amino acid changes); deletions (differences in which one or more nucleotides or amino acids are missing, up to and including a deletion of the entire coding sequence of a gene); frameshift mutations (differences in which deletion of a number of nucleotides indivisible by 3 results in an alteration of the amino acid sequence. A mutation that results in a difference in an amino acid may also be called an amino acid substitution mutation. Amino acid substitution mutations may be described by the amino acid change relative to wild type at a particular position in the amino acid sequence.

The terms “protein”, “peptide”, “polypeptide”, and “amino acid sequence” are used interchangeably herein to refer to polymers of amino acid residues of any length. The polymer can be linear or branched, it may comprise modified amino acids or amino acid analogs, and it may be interrupted by chemical moieties other than amino acids. The terms also encompass an amino acid polymer that has been modified naturally or by intervention; for example disulfide bond formation, glycosylation, lipidation, acetylation, phosphorylation, or any other manipulation or modification, such as conjugation with a labeling, radioactive or bioactive component.

The term “sample” or “biological sample” refers to a biological specimen obtained from a subject, such as a cell, fluid of tissue sample. In some cases, biological samples contain genomic DNA, RNA (including mRNA and microRNA), protein, or combinations thereof. Examples of samples include, but are not limited to, saliva, blood, serum, plasma, platelets, urine, fecal water, spinal fluid (such as cerebrospinal fluid (CSF)), tissue biopsy, surgical specimen, cells (such as PBMCs, white blood cells, lymphocytes, or other cells of the immune system) and autopsy material.

The terms “subject”, “individual,” and “patient” are used interchangeably herein, and refer to an animal, for example a mammal, primate or human, and include all mammals, such as e.g., non-human primate, (particularly higher primates), cattle, sheep, dog, rodent (e.g., mouse or rat), guinea pig, goat, pig, cat, rabbit, cow, horse. In particular, these terms refer to a human.

As used herein, the term “treatment” refers to an intervention that ameliorates a sign or symptom of a disease or pathological condition. The terms “treating” or “treatment” or “alleviation” also refer to therapeutic treatment, excluding prophylactic or preventative measures; wherein the object is to slow down (lessen) the targeted pathologic condition or disorder. Those in need of treatment include those already with the disorder as well those suspected to have the disorder. A subject is successfully “treated” for the targeted pathologic condition or disorder if, after receiving a therapeutic amount of the treatment, said subject shows observable and/or measurable reduction in or absence of one or more of the symptoms associated with the specific disease or condition, reduced morbidity and mortality, and/or improvement in quality-of-life issues. The above parameters for assessing successful treatment and improvement in the disease are readily measurable by routine procedures familiar to a physician. As used herein, the terms “treatment”, “treat” and “treating,” with reference to a disease, pathological condition or symptom, further refers to any observable beneficial effect of the treatment. The beneficial effect can be evidenced, for example, by a delayed onset of clinical symptoms of the disease in a susceptible subject, a reduction in severity of some or all clinical symptoms of the disease, a slower progression of the disease, a reduction in the number of relapses of the disease, an improvement in the overall health or well-being of the subject, or by other parameters well known in the art that are specific to the particular disease. A therapeutic treatment is a treatment administered to a subject after signs and symptoms of the disease have developed.

The expression “method of treating cancer expressing the O-acetylated-GD2 ganglioside” refer to curing, reversing, attenuating, alleviating, minimizing, suppressing or halting the deleterious effects of a cancer expressing the O-acetylated-GD2 ganglioside or cancer expressing the O-acetylated-GD2 ganglioside progression or attenuating the progression of a cancer expressing the O-acetylated-GD2 ganglioside. Preferably, such treatment also leads to the regression of tumor growth or metastasis spread, i.e., the decrease in size of a measurable tumor. Most preferably, such treatment leads to the complete regression of the tumor.

The term “therapeutic” refers to a treatment administered to a subject who exhibit early or established signs of a disease. The term “curative” refers to a treatment administered to a subject suffering from a disease for the purpose of curing the disease, i.e., of making any sign of the disease disappear or becoming undetectable.

The term “therapeutically effective amount” refers to an amount effective, at dosages and for periods of time necessary, to achieve a desired therapeutic or biological effect.

### DETAILED DESCRIPTION

CASD1 typically refers to the protein referenced as Q96PB1 in the UniProtKB/Swiss-Prot database on Jun. 2, 2021.

Alternative names for CASD1 include “CAS1 domain-containing protein 1” and “N-acetylneuraminate 9-O-acetyltransferase”, as non-limiting examples. As used herein, the expressions “CASD1” and “CAS1 domain-containing protein 1” and “N-acetylneuraminate 9-O-acetyltransferase” are used indifferently.

In the NCBI databases (https://www.ncbi.nlm.nih.gov), the reference CASD1 gene sequence corresponds to NCBI Gene ID: 64921, as updated as of Jun. 8, 2021. The reference CASD1 human protein sequence corresponds to SEQ ID NO: 1. In the context of the invention, CASD1 refers to any sequence corresponding to SEQ ID NO: 1 in other species.

The human CASD1 gene consists of 18 exons on chromosome 7q21.3. The major transcript encompasses 3942 nucleotides and encodes a 797 amino-acid protein composed of an N-terminal serine-glycine-asparagine-histidine (SGNH) hydrolase-fold domain that harbors a catalytic triad and a C-terminal multipass transmembrane domain. CASD1 is localized in the Golgi apparatus with its SGNH domain facing the Golgi lumen.

GD2 typically refers to a disialoganglioside whose structure is shown on FIG. 7A.

Alternative names for GD2 include “GD2 disialoganglioside” and “G2 ganglioside”, as non-limiting examples. The expressions “GD2”, “GD2 disialoganglioside” and “G2 ganglioside” are herein used indifferently.

GD2 typically refers to a disialoganglioside notably expressed on tumors of neuroectodermal origin, including human neuroblastoma and melanoma.

OAcGD2 typically refers to an O-acetylated form of the disialoganglioside GD2. Two exemplary structures of OAcGD2 are shown on FIGS. 7B and 7C.

Alternative names for OAcGD2 include “O-acetylated-GD2 ganglioside”, “O-acetyl GD2 ganglioside” and “O-acetyl GD2”, as non-limiting examples. The expressions “OAcGD2”, “O-acetylated-GD2 ganglioside”, “O-acetyl GD2 ganglioside” and “O-acetyl GD2” are herein used indifferently.

OAcGD2 typically refers to the O-acetylated derivative of GD2 ganglioside (OAcGD2) and is expressed in cancer tissues.

GD2 synthase typically refers to the protein referenced as Q00973 in the UniProtKB/Swiss-Prot database on Jun. 2, 2021.

Alternative names for GD2 synthase include “GD2S”, “Beta-1,4 N-acetyl-galactosaminyltransferase 1”, “(N-acetylneuraminyl)-galactosylglucosylceramide”, “GalNAc-T” and “B4GALNT1”, as non-limiting examples. The expressions “GD2 synthase”, “GD2S”, “Beta-1,4 N-acetyl-galactosaminyltransferase 1”, “(N-acetylneuraminyl)-galactosylglucosylceramide”, “GalNAc-T” and “B4GALNT1” are herein used indifferently.

Typically, GD2 synthase is an enzyme involved in the biosynthesis of gangliosides GM2, GD2, GT2 and GA2 from GM3, GD3, GT3 and GA3, respectively.

The reference B4GALNT1 gene sequence corresponds to NCBI Gene ID: 2583, as updated as of Jun. 8, 2021. The reference GD2 synthase human protein sequence corresponds to SEQ ID NO: 2. In the context of the invention, GD2 synthase refers to any sequence corresponding to SEQ ID NO: 2 in other species.

GD3 synthase typically refers to the protein referenced as Q92185 in the UniProtKB/Swiss-Prot database on Jun. 2, 2021.

Alternative names for GD3 synthase include “GD3S”, “Alpha-N-acetylneuraminide alpha-2,8-sialyltransferase”, “Alpha-2,8-sialyltransferase 8A” “SIAT8-A” and “ST8SIA I”, as non-limiting examples. The expressions “GD3 synthase”, “GD3S”, “Alpha-N-acetylneuraminide alpha-2,8-sialyltransferase”, “Alpha-2,8-sialyltransferase 8A” “SIAT8-A” and “ST8SIA I” are herein used indifferently.

Typically, GD3 synthase catalyzes the addition of sialic acid in alpha 2,8-linkage to the sialic acid moiety of the ganglioside GM3 to form ganglioside GD3.

The reference ST8SIA I gene sequence corresponds to NCBI Gene ID: 6489, as updated as of Jun. 8, 2021. The reference GD3 synthase human protein sequence corresponds to SEQ ID NO: 3. In the context of the invention, GD3 synthase refers to any sequence corresponding to SEQ ID NO: 3 in other species.

CERK typically refers to the protein referenced as Q8TCT0 in the UniProtKB/Swiss-Prot database on Jun. 2, 2021.

Alternative names for CERK include “Ceramide kinase”, “acylsphingosine kinase”, “lipid kinase 4” and “LK4”, as non-limiting examples. The expressions “CERK” and “Ceramide kinase”, “acylsphingosine kinase”, “lipid kinase 4” and “LK4” are herein used indifferently.

Typically, CERK is an enzyme that catalyzes the phosphorylation of ceramide to form ceramide 1-phosphate.

The reference CERK gene sequence corresponds to NCBI Gene ID: 64781, as updated as of Jun. 8, 2021. The reference CERK human protein sequence corresponds to SEQ ID NO: 4. In the context of the invention, CERK refers to any sequence corresponding to SEQ ID NO: 4 in other species.

PIK3C2A typically refers to the protein referenced as O00443 in the UniProtKB/Swiss-Prot database on Jun. 2, 2021.

Alternative names for PIK3C2A include “Phosphatidylinositol 4-phosphate 3-kinase C2 domain-containing subunit alpha”, “PI3K-C2-alpha”, “PtdIns-3-kinase C2 subunit alpha” and “Phosphoinositide 3-kinase-C2-alpha” as non-limiting examples. The expressions “PIK3C2A” and “Phosphatidylinositol 4-phosphate 3-kinase C2 domain-containing subunit alpha”, “PI3K-C2-alpha”, “PtdIns-3-kinase C2 subunit alpha” and “Phosphoinositide 3-kinase-C2-alpha” are herein used indifferently.

Typically, PIK3C2A generates phosphatidylinositol 3-phosphate (PtdIns3P) and phosphatidylinositol 3,4-bisphosphate (PtdIns(3,4)P2) that act as second messengers.

The reference PIK3C2A gene sequence corresponds to NCBI Gene ID: 5286, as updated as of Jun. 8, 2021. The reference PIK3C2A human protein sequence corresponds to SEQ ID NO: 5. In the context of the invention, PIK3C2A refers to any sequence corresponding to SEQ ID NO: 5 in other species.

PDK3 typically refers to the protein referenced as Q15120 in the UniProtKB/Swiss-Prot database on Jun. 2, 2021.

Alternative names for PDK3 include “[Pyruvate dehydrogenase (acetyl-transferring)] kinase isozyme 3” and “Pyruvate dehydrogenase kinase isoform 3”, as non-limiting examples. The expressions “PDK3” and “[Pyruvate dehydrogenase (acetyl-transferring)] kinase isozyme 3” and “Pyruvate dehydrogenase kinase isoform 3” are herein used indifferently.

Typically, PDK3 inhibits pyruvate dehydrogenase activity by phosphorylation of the El subunit PDHA1, and thereby regulates glucose metabolism and aerobic respiration.

The reference PDK3 gene sequence corresponds to NCBI Gene ID: 5165, as updated as of Jun. 8, 2021. The reference PDK3 human protein sequence corresponds to SEQ ID NO: 6. In the context of the invention, PDK3 refers to any sequence corresponding to SEQ ID NO: 6 in other species.

MERTK typically refers to the protein referenced as Q12866 in the UniProtKB/Swiss-Prot database on Jun. 2, 2021.

Alternative names for MERTK include “Tyrosine-protein kinase Mer”, “proto-oncogene c-Mer” and “receptor tyrosine kinase MerTK”, as non-limiting examples. The expressions “MERTK” and “Tyrosine-protein kinase Mer”, “proto-oncogene c-Mer” and “receptor tyrosine kinase MerTK” are herein used indifferently.

Typically, MERTK is a receptor tyrosine kinase that transduces signals from the extracellular matrix into the cytoplasm by binding to several ligands including LGALS3, TUB, TULP1 or GAS6.

The reference MERTK gene sequence corresponds to NCBI Gene ID: 10461, as updated as of Jun. 8, 2021. The reference MERTK human protein sequence corresponds to SEQ ID NO: 7. In the context of the invention, MERTK refers to any sequence corresponding to SEQ ID NO: 7 in other species.

NME3 typically refers to the protein referenced as Q13232 in the UniProtKB/Swiss-Prot database on Jun. 2, 2021.

Alternative names for NME3 include “Nucleoside diphosphate kinase 3”, “NDK 3”, “NDP kinase 3”, “DR-nm23”, “Nucleoside diphosphate kinase C”, “NDPKC” and “nm23-H3” as non-limiting example. The expressions “NME3” and “Nucleoside diphosphate kinase 3”, “NDK 3”, “NDP kinase 3”, “DR-nm23”, “Nucleoside diphosphate kinase C”, “NDPKC” and “nm23-H3” are herein used indifferently.

Typically, NME3 has a major role in the synthesis of nucleoside triphosphates other than ATP.

The reference NME3 gene sequence corresponds to NCBI Gene ID: 4832, as updated as of Jun. 8, 2021. The reference NME3 human protein sequence corresponds to SEQ ID NO: 8. In the context of the invention, NME3 refers to any sequence corresponding to SEQ ID NO: 8 in other species.

EZH2 typically refers to the protein referenced as NP_004447.2 in the NCBI databases on Jun. 8, 2022 (corresponding to the protein of sequence SEQ ID NO: 53), or any of the isoforms NP_694543.1, NP_001190176.1, NP_001190177.1 and NP_001190178.1.

Alternatives names for EZH2 include “Enhancer Of Zeste 2 Polycomb Repressive Complex 2 Subunit”, “ENX-1”, KMT6″, KMT6A″, “Histone-Lysine N-Methyltransferase EZH2”, “Lysine N-Methyltransferase 6”, “Enhancer Of Zeste Homolog 2”, “WVS”, “ENX1”, “WVS2”, and “EC 2.1.1.43”, which are herein used indifferently.

Typically, EZH2 is a histone-lysine N-methyltransferase enzyme that participates in histone methylation, for instance in histone H3 lysine 27 methylation, and transcriptional repression.

The reference EZH2 gene sequence corresponds to NCBI Gene ID 2146, as updated on Jun. 5, 2022. The reference EZH2 human protein sequence corresponds to SEQ ID NO: 53. In the context of the invention, EZH2 refers to any sequence corresponding to SEQ ID NO: 53 in other species.

In the context of the present invention, a “subject” refers to a warm-blooded animal, preferably a mammal. The term “mammal” refers here to any mammal, including humans, domestic and farm animals, and zoo, sports, or pet animals, such as dogs, cats, cattle, horses, sheep, pigs, goats, rabbits, rats, mice etc. Preferably, the mammal is a primate (such as a chimpanzee). More preferably, the subject is a human.

The subject according to the invention may be a male or a female, of any age. Thus, adults, children and newborn subjects, either male or female, are encompassed.

In some embodiments, a subject may be a “patient”, i.e., a subject who/which is monitored for the development of a disease; or is receiving, or is awaiting the receipt of, medical care; or was/is/will be the object of a medical procedure.

In some embodiments, in the context of the invention, the subject suffers from, and/or has been diagnosed as suffering from, a cancer expressing the O-acetylated-GD2 ganglioside.

As used herein, the term “biological sample” means a substance of biological origin, such as a cell, fluid of tissue specimen. Examples of biological samples include blood and components thereof such as serum, plasma, platelets, lymph, saliva, urine, fecal water, spinal fluid, and samples obtained by biopsy of a normal or abnormal (e.g., tumorous) organ or tissue, such as a tissue sample or a cell sample obtained from a biopsy.

Preferably, a biological sample according to the present invention is a blood sample, a serum sample, a plasma sample, a urine sample, a tissue sample from a biopsy or a cell sample from a biopsy. The biological sample according to the invention may be obtained from the subject by any appropriate means of sampling known from the skilled person.

Biological samples may contain genomic DNA, circulating free DNA, RNA (including mRNA, microRNA . . . ), proteins, or combinations thereof.

As used herein, the terms “cancer expressing the O-acetylated-GD2 ganglioside” or “cancer expressing OAcGD2” refer to a cancer comprising cells expressing the O-acetylated form of GD2 ganglioside. In particular, the O-acetylated form of GD2 ganglioside may be expressed on the cell surface of the cancer cells

In the context of the invention, the cancer expressing the O-acetylated-GD2 ganglioside is preferably characterized by the presence of cells expressing the O-acetylated-GD2 ganglioside at their cell surface in the subject.

Typically, said cells express more than 1,000 O-acetylated-GD2 ganglioside molecules on their cell surface, preferably more than 10,000, and more preferably more than 50,000 O-acetylated-GD2 ganglioside molecules on their cell surface. More generally, the term “cancer expressing the O-acetylated-GD2 ganglioside” refers to a cancer presenting more than 10% of cells expressing the O-acetylated-GD2 ganglioside, preferably more than 15%, and still more preferably more than 20%. Preferably, said cells are Cancer Stem Cells (CSCs).

Said cancer expressing the O-acetylated-GD2 ganglioside may for instance be a neuroblastoma, glioma (including glioblastoma), retinoblastoma, Ewing's family of tumors, sarcoma (including rhabdomyosarcoma, osteosarcoma, leiomyosarcoma, liposarcoma, and fibrosarcoma), lung cancer (including small cell lung cancer), breast cancer, melanoma (including uveal melanoma), metastatic renal carcinoma, head and neck cancer, hematological cancers (including leukemia, Hodgkin lymphoma, non-Hodgkin lymphoma and myeloma), colorectal cancer, pancreatic cancer, prostate cancer, liver cancer, bladder cancer, gastric/stomach cancer, cervical cancer, endometrial cancer, neuroendocrine cancer, esophageal cancer, ovarian cancer, skin cancer, kidney cancer, soft tissue sarcoma, adrenal cancer, testicular cancer, thymic cancer (including thymoma) or thyroid cancer.

In some embodiments, said cancer expressing the O-acetylated-GD2 ganglioside is selected from the group consisting of neuroblastoma, glioma (including glioblastoma), retinoblastoma, Ewing's family of tumors, sarcoma (including rhabdomyosarcoma, osteosarcoma, leiomyosarcoma, liposarcoma, and fibrosarcoma), lung cancer (including small cell lung cancer), breast cancer, melanoma (including uveal melanoma), metastatic renal carcinoma, head and neck cancer, hematological cancers (including leukemia, Hodgkin lymphoma, non-Hodgkin lymphoma and myeloma), colorectal cancer, pancreatic cancer, prostate cancer, liver cancer, bladder cancer, gastric/stomach cancer, cervical cancer, endometrial cancer, neuroendocrine cancer, esophageal cancer, ovarian cancer, skin cancer, kidney cancer, soft tissue sarcoma, adrenal cancer, testicular cancer, thymic cancer (including thymoma) and thyroid cancer.

In some embodiments, the cancer expressing the O-acetylated-GD2 ganglioside is adrenocortical carcinoma, cholangiocarcinoma, bladder urothelial carcinoma, gliomas, breast invasive carcinoma, cervical squamous cell carcinoma and endocervical adenocarcinoma, colon and rectum adenocarcinoma, esophageal carcinoma, uveal melanoma, head and neck squamous cell carcinoma, acute myeloid leukemia, kidney PAN cancer, liver hepatocellular carcinoma, lung adenocarcinoma, lung squamous cell carcinoma, ovarian serous cystadenocarcinoma, pancreatic adenocarcinoma, prostate adenocarcinoma, skin cutaneous melanoma, stomach adenocarcinoma, testicular germ cell tumors, thymoma, thyroid carcinoma, uterine corpus endometrial carcinoma, pheochromocytoma and paraganglioma or sarcoma.

In some embodiments, the cancer expressing the O-acetylated-GD2 ganglioside is breast cancer.

The inventors surprisingly showed that CASD1 is essential for the biosynthesis of the O-acetylated-GD2 ganglioside.

Thus, the present invention firstly relates to the use of CASD1 as a biomarker of a cancer expressing the O-acetylated-GD2 ganglioside.

CASD1 may advantageously be used as a biomarker of a cancer expressing the O-acetylated-GD2 ganglioside in combination with at least one biomarker selected from the group consisting of OAcGD2, GD2, GD2S, GD3S, CERK, PIK3C2A, PDK3, MERTK and NME3.

Thus, in some embodiments, the invention relates to the use of CASD1, and at least one marker selected from the group consisting of OAcGD2, GD2, GD2S, GD3S, CERK, PIK3C2A, PDK3, MERTK and NME3, as biomarkers of a cancer expressing the O-acetylated-GD2 ganglioside.

The present invention further concerns an in vitro method of diagnosing a cancer expressing the O-acetylated-GD2 ganglioside in a subject, said method comprising the steps of:
a1) measuring CASD1 expression level as a biomarker in a biological sample of the subject; and
b) based on the level measured at step a1), diagnosing a cancer expressing the O-acetylated-GD2 ganglioside in said subject.

In some embodiments, the invention relates to an in vitro method of diagnosing a cancer expressing the O-acetylated-GD2 ganglioside in a subject, said method comprising the steps of:
a1) measuring CASD1 expression level as a biomarker in a biological sample of the subject;
a1′) optionally, measuring the expression level of at least one biomarker selected from the group consisting of OAcGD2, GD2, GD2S, GD3S, CERK, PIK3C2A, PDK3, MERTK and NME3, in a biological sample of the subject; and
b) based on the levels measured at step a1), and optionally at step a1′), diagnosing a cancer expressing the O-acetylated-GD2 ganglioside in said subject.

The present invention also concerns an in vitro method of selecting a subject suffering from a cancer expressing the O-acetylated-GD2 ganglioside for treatment targeting said cancer, said method comprising the steps of:
a1) measuring CASD1 expression level as a biomarker in a biological sample of the subject; and
b) based on the level measured at step a1), selecting said subject to undergo treatment targeting said cancer expressing the O-acetylated-GD2 ganglioside.

In some embodiments, the invention relates to an in vitro method of selecting a subject suffering from a cancer expressing the O-acetylated-GD2 ganglioside for treatment targeting said cancer, said method comprising the steps of:
a1) measuring CASD1 expression level as a biomarker in a biological sample of the subject;
a1′) optionally, measuring the expression level of at least one biomarker selected from the group consisting of OAcGD2, GD2, GD2S, GD3S, CERK, PIK3C2A, PDK3, MERTK and NME3, in a biological sample of the subject; and
b) based on the levels measured at step a1), and optionally at step a1′), selecting said subject to undergo treatment targeting said cancer expressing the O-acetylated-GD2 ganglioside.

As used herein, the term “expression” may refer alternatively to the presence of circulating free DNA, to the transcription of CASD1 gene (i.e., expression of the RNA) or to the translation of CASD1 (i.e., expression of the protein), or to the presence of the CASD1 protein within a cell.

Methods for determining the expression level are well-known from the skilled artisan, and include, without limitation, determining the transcriptome (in an embodiment wherein expression relates to transcription of CASD1 gene) or proteome (in an embodiment wherein expression relates to translation of CASD1) of a cell.

In some embodiments, the expression of CASD1 is assessed at the DNA level. For instance, the expression of circulating tumor DNA (ctDNA) may be assessed.

In another embodiment, the expression of CASD1 is assessed at the RNA level. Methods for assessing the transcription level of a gene are well known in the art. Examples of such methods include, but are not limited to, qPCR, RT-PCR, RT-qPCR, Northern Blot, hybridization techniques such as, for example, use of microarrays, and combination thereof including but not limited to, hybridization of amplicons obtained by RT-PCR, sequencing such as, for example, next-generation DNA sequencing (NGS) or RNA-seq (also known as “Whole Transcriptome Shotgun Sequencing”) and the like.

Examples of PCR or qPCR primers that may be used for assessing the expression of CASD1 include, but are not limited to, the following couples of primers: Forward primer 1:
5′-GCTCGGGATCCGCGGCTCTGGCCTACAACCTG-3′ (SEQ ID NO: 9) and Reverse primer 1:
5′-GCTCGCTCGAGATGTTTTGATTTATCTTGAATGGATG-3′ (SEQ ID NO: 10), or
Forward primer 2: 5′-ATGTTCACAACGCCACGG-3′ (SEQ ID NO: 27) and Reverse primer 2: 5′-CAGGAACCATCCACAGGC-3′ (SEQ ID NO: 28), or
Forward primer 3: 5′-GTGGATTTTCTGTGGCATCC-3′ (SEQ ID NO: 31) and Reverse primer 3: 5′-AAGCGCTTCACTGCTACCAT-3′ (SEQ ID NO: 32).

In some embodiments, the expression of CASD1 is assessed at the protein level. Methods for determining a protein level in a sample are well-known in the art. Examples of such methods include, but are not limited to, mass spectrometry, immunohistochemistry, Multiplex methods (Luminex), western blot, enzyme-linked immunosorbent assay (ELISA), sandwich ELISA, fluorescent-linked immunosorbent assay (FLISA), enzyme immunoassay (EIA), radioimmunoassay (RIA), flow cytometry (FACS) and the like.

In some embodiments, determining the expression level of CASD1 specifically corresponds to the detection and quantification of the CASD1 protein within a cell. Methods for analyzing the presence of a protein in a cell are well-known to the skilled artisan and include, without limitation, FACS analysis, immunohistochemistry, mass spectrometry, western blot associated with cell fractionation, enzyme-linked immunosorbent assay (ELISA), sandwich ELISA, fluorescent-linked immunosorbent assay (FLISA), enzyme immunoassay (EIA), radioimmunoassay (RIA) or image analysis, for example high content analysis and the like.

The detection or quantification of CASD1 may be done by using an anti-CASD1 antibody, such as e.g., the C7orf12 anti-CASD1 rabbit polyclonal antibody.

In a particular embodiment, the method of diagnosing a cancer expressing the O-acetylated-GD2 ganglioside, the method of selecting a subject suffering from a cancer expressing the O-acetylated-GD2 ganglioside for treatment targeting said cancer, or the method of monitoring the response of a subject suffering from a cancer expressing the O-acetylated-GD2 ganglioside to a treatment targeting said cancer, further comprises a step a2) of comparing the expression level at step a1), and/or optionally at step a1′), with a threshold value.

Preferably, the threshold value corresponds to a normal expression level of the biomarker of interest; such as e.g., normal CASD1 expression level for step a1), or normal expression level of the at least one biomarker selected from the group consisting of OAcGD2, GD2, GD2S, GD3S, CERK, PIK3C2A, PDK3, MERTK and NME3 for step a1′).

As intended herein a “normal expression level” of a biomarker, e.g., CASD1, means that the expression level of the biomarker, e.g., CASD1, in the biological sample is within the norm cut-off values for that gene or protein. The norm is dependent on the biological sample type and on the method used for measuring the biomarker expression level, e.g., CASD1 expression level, in the biological sample. In particular, the threshold value may be the biomarker expression level, e.g., CASD1 expression level, that gives a negative predictive value and a positive predictive value superior to 10%, 20%, 30%, 40%, 50%, 60%, 70% or 80%, preferably superior to 85%, more preferably superior to 90%, even more preferably superior to 95% in the targeted population.

As used herein, the term “targeted population” refers to a population constituted of subjects who share certain biological parameters such as e.g., gender, age group, or certain environmental parameters such as e.g., geographical region.

In some embodiments, the threshold value is the biomarker expression level, e.g., CASD1 expression level, measured in a population of healthy subject.

Preferably, in the methods of the invention, it is further determined whether the measured expression level is increased or decreased compared to the threshold value according to the invention. Still preferably, in the methods of the invention, it is further determined the level of increase or decrease of the measured expression level compared to the threshold value according to the invention.

As used herein, the expression “level of increase” means the percentage of increase of the measured expression level compared to the threshold value according to the invention or the number of folds of increase of the measured expression level compared to the threshold value according to the invention.

Preferably, when the measured expression level is increased compared to the threshold value, the measured expression level is significantly higher than the threshold value.

Also preferably, when the measured expression level is decreased compared to the threshold value, the measured expression level is significantly lower than the threshold value.

The inventors demonstrated that the increase of CASD1 expression level enabled diagnosing a cancer expressing the O-acetylated-GD2 ganglioside.

In some embodiments, the subject is diagnosed as suffering from a cancer expressing the O-acetylated-GD2 ganglioside, if the expression level measured at step a1), and/or optionally at step a1′), is higher than the threshold value.

Advantageously, CASD1 may be combined with a at least one biomarker selected from the group consisting of OAcGD2, GD2, GD2S, GD3S, CERK, PIK3C2A, PDK3, MERTK and NME3, as biomarkers of a cancer expressing the O-acetylated-GD2 ganglioside in the subject. Then, an increase of the expression levels of the at least two biomarkers in a biological sample of a subject compared to the threshold values enabled diagnosing a cancer expressing the O-acetylated-GD2 ganglioside.

Accordingly, in the methods for diagnosing a cancer expressing the O-acetylated-GD2 ganglioside according to the invention, an expression level measured at step a1), and optionally at step a1′), which is higher than the threshold value is indicative of the presence of a cancer expressing the O-acetylated-GD2 ganglioside in the subject.

Also, in the methods for diagnosing a cancer expressing the O-acetylated-GD2 ganglioside, an expression level measured at step a1), and optionally at step a1′), which is lower than the threshold value is preferably indicative of an absence of cancer expressing the O-acetylated-GD2 ganglioside in the subject.

Preferably, in the methods for selecting a subject suffering from a cancer expressing the O-acetylated-GD2 ganglioside for treatment targeting said cancer according to the invention, the subject is selected to undergo treatment targeting said cancer expressing the O-acetylated-GD2 ganglioside if the expression level measured at step a1), and optionally at step a1′), is higher than the threshold value.

In a particular embodiment, the method of diagnosing a cancer expressing the O-acetylated-GD2 ganglioside in a subject further comprises a step c) of submitting the subject to a treatment targeting a cancer expressing the O-acetylated-GD2 ganglioside, if a cancer expressing the O-acetylated-GD2 ganglioside has been diagnosed in step b).

In a particular embodiment, the method of selecting a subject suffering from a cancer expressing the O-acetylated-GD2 ganglioside for treatment targeting said cancer, further comprises a step c) of submitting the subject to a treatment targeting a cancer expressing the O-acetylated-GD2 ganglioside, if said subject is selected to undergo treatment targeting said cancer expressing the O-acetylated-GD2 ganglioside in step b).

According to another aspect, the invention relates to a method of diagnosing a cancer expressing the O-acetylated-GD2 ganglioside in a subject and treating said subject, said method comprising the steps of:
a1) measuring CASD1 expression level as a biomarker in a biological sample of the subject;
b) based on the level measured at step a1), diagnosing a cancer expressing the O-acetylated-GD2 ganglioside in said subject; and
c) administering a treatment targeting the cancer expressing the O-acetylated-GD2 ganglioside to said subject.

In some embodiments, the invention relates to a method of diagnosing a cancer expressing the O-acetylated-GD2 ganglioside in a subject and treating said subject, said method comprising the steps of:
a1) measuring CASD1 expression level as a biomarker in a biological sample of the subject;
a1′) optionally, measuring the expression level of at least one biomarker selected from the group consisting of OAcGD2, GD2, GD2S, GD3S, CERK, PIK3C2A, PDK3, MERTK and NME3, in a biological sample of the subject;
b) based on the level measured at step a1), and optionally at step a1′), diagnosing a cancer expressing the O-acetylated-GD2 ganglioside in said subject; and
c) administering a treatment targeting the cancer expressing the O-acetylated-GD2 ganglioside to said subject.

The inventors demonstrated that CASD1 expression level in the biological sample of a subject may be useful in the diagnosis of a cancer expressing the O-acetylated-GD2 ganglioside. Subjects who have been diagnosed as suffering from a cancer expressing the O-acetylated-GD2 ganglioside may further be selected for treatment targeting said cancer. They may also further benefit from an appropriate monitoring of their response to said treatment targeting cancer.

Accordingly, another aspect of the present invention is a method of monitoring the response of a subject suffering from a cancer expressing the O-acetylated-GD2 ganglioside to a treatment targeting said cancer, said method comprising the steps of:
a1) measuring CASD1 expression level as a biomarker in a biological sample of the subject; and
b) based on the level measured at step a1), monitoring the response of said subject to said treatment.

In some embodiments, said method of monitoring the response of a subject suffering from a cancer expressing the O-acetylated-GD2 ganglioside to a treatment targeting said cancer, said method comprising the steps of:
a1) measuring CASD1 expression level as a biomarker in a biological sample of the subject;
a1′) optionally, measuring the expression level of at least one biomarker selected from the group consisting of OAcGD2, GD2, GD2S, GD3S, CERK, PIK3C2A, PDK3, MERTK and NME3, in a biological sample of the subject; and
b) based on the level measured at step a1), and optionally at step a1′), monitoring the response of said subject to said treatment.

The expression “monitoring the response of a subject to a treatment targeting a cancer expressing the O-acetylated-GD2 ganglioside” may for instance mean adapting the treatment. Preferably, “monitoring the response of a subject to a treatment targeting a cancer expressing the O-acetylated-GD2 ganglioside” means changing the drug used to treat the subject, or increasing or reducing the dose, the administration frequency, or changing the administration route of the treatment.

Another aspect of the present invention is a method of monitoring the progression of a cancer expressing the O-acetylated-GD2 ganglioside in a subject, said method comprising the steps of:
a1) measuring CASD1 expression level as a biomarker in a biological sample of the subject; and
b) based on the level measured at step a1), monitoring the progression of said cancer expressing the O-acetylated-GD2 ganglioside in said subject.

In some embodiments, said method of monitoring the progression of a cancer expressing the O-acetylated-GD2 ganglioside in a subject comprises the steps of:
a1) measuring CASD1 expression level as a biomarker in a biological sample of the subject;
a1′) optionally, measuring the expression level of at least one biomarker selected from the group consisting of OAcGD2, GD2, GD2S, GD3S, CERK, PIK3C2A, PDK3, MERTK and NME3, in a biological sample of the subject; and
b) based on the level measured at step a1), and optionally at step a1′), monitoring the progression of said cancer expressing the O-acetylated-GD2 ganglioside in said subject.

When the method is used to monitor the progression of a cancer or to monitor the response of a subject to a treatment, it is repeated at least at two different points in time (e.g., before and after onset of a treatment).

Accordingly, the invention also relates to an in vitro method for monitoring the response of the subject to a treatment comprising the steps of:
a) measuring CASD1 expression level as a biomarker in a biological sample of the subject, before onset of said treatment; and
b) measuring CASD1 expression level as a biomarker in a biological sample of the subject, after onset of said treatment;
wherein a decrease in CASD1 expression level in the course of time indicates that said treatment is efficient for treating the subject.

The invention also relates to an in vitro method for monitoring the response of the subject to a treatment comprising the steps of:
a) measuring CASD1 expression level as a biomarker, and optionally the expression level of at least one biomarker selected from the group consisting of OAcGD2, GD2, GD2S, GD3S, CERK, PIK3C2A, PDK3, MERTK and NME3, in a biological sample of the subject, before onset of said treatment; and
b) measuring CASD1 expression level as a biomarker, and optionally the expression level of at least one biomarker selected from the group consisting of OAcGD2, GD2, GD2S, GD3S, CERK, PIK3C2A, PDK3, MERTK and NME3, in a biological sample of the subject, after onset of said treatment;
wherein a decrease in CASD1 expression level, and optionally in the expression level of at least one biomarker selected from the group consisting of OAcGD2, GD2, GD2S, GD3S, CERK, PIK3C2A, PDK3, MERTK and NME3, in the course of time indicates that said treatment is efficient for treating the subject.

The invention also relates to an in vitro method for monitoring the progression of a cancer expressing the O-acetylated-GD2 ganglioside comprising the steps of:
a) measuring CASD1 expression level as a biomarker in a biological sample of the subject, when monitoring is started; and
b) measuring CASD1 expression level as a biomarker in a biological sample of the subject, at a certain point in time;
wherein a decrease in CASD1 expression level in the course of time indicates a favorable cancer progression.

The invention also relates to an in vitro method for monitoring the progression of a cancer expressing the O-acetylated-GD2 ganglioside comprising the steps of:
a) measuring CASD1 expression level as a biomarker, and optionally the expression level of at least one biomarker selected from the group consisting of OAcGD2, GD2, GD2S, GD3S, CERK, PIK3C2A, PDK3, MERTK and NME3, in a biological sample of the subject, when monitoring is started; and
b) measuring CASD1 expression level as a biomarker, and optionally the expression level of at least one biomarker selected from the group consisting of OAcGD2, GD2, GD2S, GD3S, CERK, PIK3C2A, PDK3, MERTK and NME3, in a biological sample of the subject, at a certain point in time;
wherein a decrease in CASD1 expression level, and optionally in the expression level of at least one biomarker selected from the group consisting of OAcGD2, GD2, GD2S, GD3S, CERK, PIK3C2A, PDK3, MERTK and NME3, in the course of time indicates a favorable cancer progression.

The monitoring of disease progression or treatment efficiency is typically performed by determining a biomarker expression level, e.g., CASD1 expression level, at different points in time, for instance at 2-week, 1-month, 2-month, 3-month intervals, etc.

A “decrease in a biomarker expression level”, e.g., a “decrease in CASD1 expression level” is evaluated by comparing said biomarker expression level, e.g., CASD1 expression level, when monitoring is started with said biomarker expression level, e.g., CASD1 expression level, at any point in time. Said decrease is preferably statistically significant. A statistically significant decrease can for example correspond to a decrease of at least 5%, 10%, 15%, 25%, 30%, 40% or 50%.

For example, the efficacy of the treatment may be evaluated by measuring a biomarker expression level, e.g., CASD1 expression level, in a “treated” subject before and after treatment and, if it is reduced by at least 5%, 10%, 20%, 30%, 40%, 50%, or 60%, more preferably by at least about 70%, even more preferably by at least 75%, 80%, 85%, 90%, 95%, 98% or 99%, or even more (99.5%, 99.8%, 99.9% or 100%), then the treatment is considered as effective.

In some embodiments, the method of selecting a subject suffering from a cancer expressing the O-acetylated-GD2 ganglioside for treatment targeting said cancer further comprises the following steps:
a1′) measuring the expression level of at least one biomarker selected from the group consisting of OAcGD2, GD2, GD2S, GD3S, CERK, PIK3C2A, PDK3, MERTK and NME3, in a biological sample of the subject; and
b′) based on the levels measured at step a1) and step a1′), selecting said subject to undergo treatment targeting said cancer expressing the O-acetylated-GD2 ganglioside.

In some embodiments, the method of monitoring the response of a subject suffering from a cancer expressing the O-acetylated-GD2 ganglioside, to a treatment targeting said cancer, further comprises the following steps:
a1′) measuring the expression level of at least one biomarker selected from the group consisting of OAcGD2, GD2, GD2S, GD3S, CERK, PIK3C2A, PDK3, MERTK and NME3, in a biological sample of the subject; and
b′) based on the levels measured at step a1) and step a1′), monitoring the response of said subject to said treatment.

In some embodiments, the method of diagnosing a cancer expressing the O-acetylated-GD2 ganglioside in a subject further comprises the following steps:
a1′) measuring the expression level of at least one biomarker selected from the group consisting of OAcGD2, GD2, GD2S, GD3S, CERK, PIK3C2A, PDK3, MERTK and NME3, in a biological sample of the subject; and
b′) based on the levels measured at step a1) and step a1′), diagnosing a cancer expressing the O-acetylated-GD2 ganglioside in said subject.

In some embodiments, the method of selecting a subject suffering from a cancer expressing the O-acetylated-GD2 ganglioside for treatment targeting said cancer comprises the following steps:
a1) measuring CASD1 expression level as a biomarker in a biological sample of the subject;
a1′) optionally, measuring the expression level of at least one biomarker selected from the group consisting of OAcGD2, GD2, GD2S, GD3S, CERK, PIK3C2A, PDK3, MERTK and NME3, in a biological sample of the subject; and
b′) based on the level measured at step a1), and optionally at step a1′), selecting said subject to undergo treatment targeting said cancer expressing the O-acetylated-GD2 ganglioside.

In some embodiments, the method of monitoring the response of a subject suffering from a cancer expressing the O-acetylated-GD2 ganglioside, to a treatment targeting said cancer, comprises the following steps:
a1) measuring CASD1 expression level as a biomarker in a biological sample of the subject;
a1′) optionally, measuring the expression level of at least one biomarker selected from the group consisting of OAcGD2, GD2, GD2S, GD3S, CERK, PIK3C2A, PDK3, MERTK and NME3, in a biological sample of the subject; and
b′) based on the level measured at step a1), and optionally at step a1′), monitoring the response of said subject to said treatment.

In some embodiments, the method of diagnosing a cancer expressing the O-acetylated-GD2 ganglioside in a subject comprises the following steps:
a1) measuring CASD1 expression level as a biomarker in a biological sample of the subject;
a1′) optionally, measuring the expression level of at least one biomarker selected from the group consisting of OAcGD2, GD2, GD2S, GD3S, CERK, PIK3C2A, PDK3, MERTK and NME3, in a biological sample of the subject; and
b′) based on the level measured at step a1), and optionally at step a1′), diagnosing a cancer expressing the O-acetylated-GD2 ganglioside in said subject.

Thus, in some embodiments, the method of selecting a subject suffering from a cancer expressing the O-acetylated-GD2 ganglioside for treatment targeting said cancer, the method of monitoring the response of a subject suffering from a cancer expressing the O-acetylated-GD2 ganglioside to a treatment targeting said cancer, or the method of diagnosing a cancer expressing the O-acetylated-GD2 ganglioside in a subject, comprises the steps of:
a1) measuring CASD1 expression level as a biomarker in a biological sample of the subject;
a1′) measuring the expression level of at least one biomarker selected from the group consisting of OAcGD2, GD2, GD2S, GD3S, CERK, PIK3C2A, PDK3, MERTK and NME3, in a biological sample of the subject; and
b′) based on the levels measured at step a1) and at step a1′), diagnosing a cancer expressing the O-acetylated-GD2 ganglioside in said subject.

Thus, in some embodiments, the method of selecting a subject suffering from a cancer expressing the O-acetylated-GD2 ganglioside for treatment targeting said cancer, the method of monitoring the response of a subject suffering from a cancer expressing the O-acetylated-GD2 ganglioside to a treatment targeting said cancer, or the method of diagnosing a cancer expressing the O-acetylated-GD2 ganglioside in a subject, comprises the steps of:
a1) measuring CASD1 and OAcGD2 expression levels in a biological sample of the subject; or
a1) measuring CASD1 and GD2 expression levels in a biological sample of the subject; or
a1) measuring CASD1 and GD2S expression levels in a biological sample of the subject; or
a1) measuring CASD1 and GD3S expression levels in a biological sample of the subject; or
a1) measuring CASD1 and CERK expression levels in a biological sample of the subject; or
a1) measuring CASD1 and PIK3C2A expression levels in a biological sample of the subject; or
a1) measuring CASD1 and PDK3 expression levels in a biological sample of the subject; or
a1) measuring CASD1 and MERTK expression levels in a biological sample of the subject; or
a1) measuring CASD1 and NME3 expression levels in a biological sample of the subject; and
b) based on the level measured at step a1), selecting said subject to undergo a treatment targeting said cancer expressing the O-acetylated-GD2 ganglioside, monitoring the response of said subject to said treatment, or diagnosing a cancer expressing the O-acetylated-GD2 ganglioside in said subject.

In some embodiments, the method of selecting a subject suffering from a cancer expressing the O-acetylated-GD2 ganglioside for treatment targeting said cancer, the method of monitoring the response of a subject suffering from a cancer expressing the O-acetylated-GD2 ganglioside to a treatment targeting said cancer, or the method of diagnosing a cancer expressing the O-acetylated-GD2 ganglioside in a subject, comprises the steps of:
a1) measuring the expression levels of CASD1 and OAcGD2, and optionally of at least one biomarker selected from the group consisting of GD2, GD2S, GD3S, CERK, PIK3C2A, PDK3, MERTK and NME3, in a biological sample of the subject; and
b) based on the levels measured at step a1), selecting said subject to undergo a treatment targeting said cancer expressing the O-acetylated-GD2 ganglioside, monitoring the response of said subject to said treatment, or diagnosing a cancer expressing the O-acetylated-GD2 ganglioside in said subject.

In some embodiments, the method of selecting a subject suffering from a cancer expressing the O-acetylated-GD2 ganglioside for treatment targeting said cancer, the method of monitoring the response of a subject suffering from a cancer expressing the O-acetylated-GD2 ganglioside to a treatment targeting said cancer, or the method of diagnosing a cancer expressing the O-acetylated-GD2 ganglioside in a subject, comprises the steps of:
a1) measuring the expression levels of CASD1 and GD2, and optionally of at least one biomarker selected from the group consisting of OAcGD2, GD2S, GD3S, CERK, PIK3C2A, PDK3, MERTK and NME3, in a biological sample of the subject; and
b) based on the levels measured at step a1), selecting said subject to undergo a treatment targeting said cancer expressing the O-acetylated-GD2 ganglioside, monitoring the response of said subject to said treatment, or diagnosing a cancer expressing the O-acetylated-GD2 ganglioside in said subject.

In some embodiments, the method of selecting a subject suffering from a cancer expressing the O-acetylated-GD2 ganglioside for treatment targeting said cancer, the method of monitoring the response of a subject suffering from a cancer expressing the O-acetylated-GD2 ganglioside to a treatment targeting said cancer, or the method of diagnosing a cancer expressing the O-acetylated-GD2 ganglioside in a subject, comprises the steps of:
a1) measuring the expression levels of CASD1 and GD2S, and optionally of at least one biomarker selected from the group consisting of OAcGD2, GD2, GD2S, GD3S, CERK, PIK3C2A, PDK3, MERTK and NME3, in a biological sample of the subject; and
b) based on the levels measured at step a1), selecting said subject to undergo a treatment targeting said cancer expressing the O-acetylated-GD2 ganglioside, monitoring the response of said subject to said treatment, or diagnosing a cancer expressing the O-acetylated-GD2 ganglioside in said subject.

In some embodiments, the method of selecting a subject suffering from a cancer expressing the O-acetylated-GD2 ganglioside for treatment targeting said cancer, the method of monitoring the response of a subject suffering from a cancer expressing the O-acetylated-GD2 ganglioside to a treatment targeting said cancer, or the method of diagnosing a cancer expressing the O-acetylated-GD2 ganglioside in a subject, comprises the steps of:
a1) measuring the expression levels of CASD1 and GD3S, and optionally of at least one biomarker selected from the group consisting of OAcGD2, GD2, GD2S, CERK, PIK3C2A, PDK3, MERTK and NME3, in a biological sample of the subject; and
b) based on the levels measured at step a1), selecting said subject to undergo a treatment targeting said cancer expressing the O-acetylated-GD2 ganglioside, monitoring the response of said subject to said treatment, or diagnosing a cancer expressing the O-acetylated-GD2 ganglioside in said subject.

In some embodiments, the method of selecting a subject suffering from a cancer expressing the O-acetylated-GD2 ganglioside for treatment targeting said cancer, the method of monitoring the response of a subject suffering from a cancer expressing the O-acetylated-GD2 ganglioside to a treatment targeting said cancer, or the method of diagnosing a cancer expressing the O-acetylated-GD2 ganglioside in a subject, comprises the steps of:
a1) measuring the expression levels of CASD1 and CERK, and optionally of at least one biomarker selected from the group consisting of OAcGD2, GD2, GD2S, GD3S, PIK3C2A, PDK3, MERTK and NME3, in a biological sample of the subject; and
b) based on the levels measured at step a1), selecting said subject to undergo a treatment targeting said cancer expressing the O-acetylated-GD2 ganglioside, monitoring the response of said subject to said treatment, or diagnosing a cancer expressing the O-acetylated-GD2 ganglioside in said subject.

In some embodiments, the method of selecting a subject suffering from a cancer expressing the O-acetylated-GD2 ganglioside for treatment targeting said cancer, the method of monitoring the response of a subject suffering from a cancer expressing the O-acetylated-GD2 ganglioside to a treatment targeting said cancer, or the method of diagnosing a cancer expressing the O-acetylated-GD2 ganglioside in a subject, comprises the steps of:
a1) measuring the expression levels of CASD1 and PIK3C2A, and optionally of at least one biomarker selected from the group consisting of OAcGD2, GD2, GD2S, GD3S, CERK, PDK3, MERTK and NME3, in a biological sample of the subject; and
b) based on the levels measured at step a1), selecting said subject to undergo a treatment targeting said cancer expressing the O-acetylated-GD2 ganglioside, monitoring the response of said subject to said treatment, or diagnosing a cancer expressing the O-acetylated-GD2 ganglioside in said subject.

In some embodiments, the method of selecting a subject suffering from a cancer expressing the O-acetylated-GD2 ganglioside for treatment targeting said cancer, the method of monitoring the response of a subject suffering from a cancer expressing the O-acetylated-GD2 ganglioside to a treatment targeting said cancer, or the method of diagnosing a cancer expressing the O-acetylated-GD2 ganglioside in a subject, comprises the steps of:
a1) measuring the expression levels of CASD1 and PDK3, and optionally of at least one biomarker selected from the group consisting of OAcGD2, GD2, GD2S, GD3S, CERK, PIK3C2A, MERTK and NME3, in a biological sample of the subject; and
b) based on the levels measured at step a1), selecting said subject to undergo a treatment targeting said cancer expressing the O-acetylated-GD2 ganglioside, monitoring the response of said subject to said treatment, or diagnosing a cancer expressing the O-acetylated-GD2 ganglioside in said subject.

In some embodiments, the method of selecting a subject suffering from a cancer expressing the O-acetylated-GD2 ganglioside for treatment targeting said cancer, the method of monitoring the response of a subject suffering from a cancer expressing the O-acetylated-GD2 ganglioside to a treatment targeting said cancer, or the method of diagnosing a cancer expressing the O-acetylated-GD2 ganglioside in a subject, comprises the steps of:
a1) measuring the expression levels of CASD1 and MERTK, and optionally of at least one biomarker selected from the group consisting of OAcGD2, GD2, GD2S, GD3S, CERK, PIK3C2A, PDK3, and NME3, in a biological sample of the subject; and
b) based on the levels measured at step a1), selecting said subject to undergo a treatment targeting said cancer expressing the O-acetylated-GD2 ganglioside, monitoring the response of said subject to said treatment, or diagnosing a cancer expressing the O-acetylated-GD2 ganglioside in said subject.

In some embodiments, the method of selecting a subject suffering from a cancer expressing the O-acetylated-GD2 ganglioside for treatment targeting said cancer, the method of monitoring the response of a subject suffering from a cancer expressing the O-acetylated-GD2 ganglioside to a treatment targeting said cancer, or the method of diagnosing a cancer expressing the O-acetylated-GD2 ganglioside in a subject, comprises the steps of:
a1) measuring the expression levels of CASD1 and NME3, and optionally of at least one biomarker selected from the group consisting of OAcGD2, GD2, GD2S, GD3S, CERK, PIK3C2A, PDK3 and MERTK, in a biological sample of the subject; and
b) based on the levels measured at step a1), selecting said subject to undergo a treatment targeting said cancer expressing the O-acetylated-GD2 ganglioside, monitoring the response of said subject to said treatment, or diagnosing a cancer expressing the O-acetylated-GD2 ganglioside in said subject.

In some embodiments, the method of diagnosing a cancer expressing the O-acetylated-GD2 ganglioside in a subject further comprises a step c) of submitting the subject to a treatment targeting a cancer expressing the O-acetylated-GD2 ganglioside, if a cancer expressing the O-acetylated-GD2 ganglioside has been diagnosed in step b).

In some embodiments, the method of selecting a subject suffering from a cancer expressing the O-acetylated-GD2 ganglioside for treatment targeting said cancer, further comprises a step c) of submitting the subject to a treatment targeting a cancer expressing the O-acetylated-GD2 ganglioside, if said subject is selected to undergo treatment targeting said cancer expressing the O-acetylated-GD2 ganglioside in step b).

In some embodiments, the invention relates to a method of diagnosing a cancer expressing the O-acetylated-GD2 ganglioside in a subject and treating said subject, said method comprising the steps of:
a1) measuring CASD1 expression level as a biomarker in a biological sample of the subject;
a1′) optionally, measuring the expression level of at least one biomarker selected from the group consisting of OAcGD2, GD2, GD2S, GD3S, CERK, PIK3C2A, PDK3, MERTK and NME3, in a biological sample of the subject;
b) based on the level measured at step a1), and optionally at step a1′), diagnosing a cancer expressing the O-acetylated-GD2 ganglioside in said subject; and
c) administering a treatment targeting the cancer expressing the O-acetylated-GD2 ganglioside to said subject.

The expression levels of CASD1 and of the at least one biomarker selected from the group consisting of OAcGD2, GD2, GD2S, GD3S, CERK, PIK3C2A, PDK3, MERTK and NME3 may be assessed in a same biological sample or in different biological samples of the subject.

The expression level of CASD1 and/or of the at least one biomarker selected from the group consisting of OAcGD2, GD2, GD2S, GD3S, CERK, PIK3C2A, PDK3, MERTK and NME3 may be assessed at the DNA level, at the RNA level or at the protein level, by the technics described hereinabove for CASD1.

The expression levels of CASD1 and of the at least one biomarker selected from the group consisting of OAcGD2, GD2, GD2S, GD3S, CERK, PIK3C2A, PDK3, MERTK and NME3 may be assessed by a same technique or by a different technique.

The expression levels of CASD1 and of the at least one biomarker selected from the group consisting of OAcGD2, GD2, GD2S, GD3S, CERK, PIK3C2A, PDK3, MERTK and NME3 may be assessed simultaneously and sequentially.

The detection or quantification of GD2S may be done by using an anti-GD2S antibody, such as e.g., the rabbit anti-B4GALNT1 polyclonal antibody (PA5-52636, Invitrogen) or the rabbit B4GALNT1/GM2 synthase polyclonal antibody (BS-12706R, Bioss). The detection or quantification of GD2S may also be done by PCR, preferably by qPCR or RT-qPCR, for instance by using primers such as the following couple of primers: Forward primer: 5′-CAGCGCTCTAGTCACGATTGC-3′ (SEQ ID NO: 33) and Reverse primer: 5′-CCACGGTAACCGTTGGGTAG-3′ (SEQ ID NO: 34).

The detection or quantification of GD3S may be done by using an anti-GD3S antibody, such as e.g., the rabbit anti-ST8SIA1 polyclonal antibody (PAB21836, Abnova). The detection or quantification of GD3S may also be done by PCR, preferably by qPCR or RT-qPCR, for instance by using primers such as the following couple of primers:

Forward primer 1:
(SEQ ID NO: 17)
5′GCTAAGCTTCGAATGGGTAAGCCTATCCCTAACCCTCTCCTCGGTCTC
GATTCTACGGGTACCAGCCCCTGCGGGCGGGC-3′
and
Reverse primer 1:
(SEQ ID NO: 18)
5′-GCTGCGGCCGCCTAGGAAGTGGGCTGGAGTG-3′,
or
Forward primer 2:
(SEQ ID NO: 35)
5′-GCGATGCAATCTCCCTCCT-3′
and
Reverse primer 2:
(SEQ ID NO: 36)
5′-TTCCCGAATTATGCTGGGAT-3′

The detection or quantification of CERK may be done by using an anti-CERK antibody, such as e.g., the rabbit anti-CERK polyclonal antibody (SAB2701099, Sigma Aldrich), or the rabbit anti-human Ceramide Kinase/CERK polyclonal antibody (LS-A4556, LSBio).

The detection or quantification of PIK3C2A may be done by using an anti- PIK3C2A antibody, such as e.g., the mouse anti-PIK3C2A monoclonal antibody Clone OTI2C2 (MA5-26506, Invitrogen) or the mouse anti-human PIK3C2A monoclonal antibody Clone 3E7 (LS-B6117, LSBio).

The detection or quantification of PDK3 may be done by using an anti-PDK3 antibody, such as e.g., the rabbit anti-PDK3 polyclonal antibody (PA5-76332, Invitrogen), or the rabbit anti-PDK3 polyclonal antibody (ab154549, Abcam), or the rabbit anti-PDK3 polyclonal antibody (HPA046583, Atlas antibodies).

The detection or quantification of MERTK may be done by using an anti- MERTK antibody, such as e.g., the rabbit recombinant anti-MERTK monoclonal antibody Clone Y323 (ab52968, Abcam).

The detection or quantification of NME3 may be done by using an anti-NME3 antibody, such as e.g., the rabbit recombinant anti-NME3 antibody Clone EPR13117 (ab181257, Abcam), or the rabbit anti-human NME3 polyclonal antibody (epitope aa51-81) (LS-C328074, LSBio).

In some embodiments, CASD1, GD2, GD2S, GD3S, CERK, PIK3C2A, PDK3, MERTK and NME3 may be used as biomarkers of the OAcGD2 expression.

In other embodiments, GD2S, GD3S, CERK, PIK3C2A, PDK3, MERTK and NME3 may be used as biomarkers of the GD2 expression.

A “treatment targeting a cancer expressing the O-acetylated-GD2 ganglioside” may for instance be an increased surveillance of said cancer, or a drug treatment.

A “treatment targeting a cancer expressing the O-acetylated-GD2 ganglioside” is preferably a drug treatment.

As used herein, “drug treatment” or “drug treatment targeting a cancer expressing the O-acetylated-GD2 ganglioside” may for instance refer to treatment with a therapy targeting the O-acetylated-GD2 ganglioside, and/or an anti-cancer agent.

In some embodiments, the “therapy targeting the O-acetylated-GD2 ganglioside” is an immunotherapy, such as e.g., an antibody, or an antigen-binding fragment, that binds to the O-acetylated-GD2 ganglioside, or a chimeric antigen receptor (CAR) grafted onto an immune effector cell (such as e.g., a T cell) comprising an antigen-binding fragment that binds to the O-acetylated-GD2 ganglioside.

Preferably, the “treatment” or “treatment targeting a cancer expressing the O-acetylated-GD2 ganglioside” comprises an antibody that binds to the O-acetylated-GD2 ganglioside or an antigen-binding fragment thereof.

As used herein, antibody-binding fragments of an antibody include, without any limitation, single chain antibodies, Fv, dsFv, Fab, Fab′, Fab′-SH, F(ab)′2, scFv, Fd, VHH, defucosylated antibodies, diabodies, triabodies and tetrabodies.

Thus, in some embodiments, the “treatment targeting a cancer expressing the O-acetylated-GD2 ganglioside” is any antibody or any antigen-binding fragment recognizing, binding or targeting the O-acetylated-GD2 ganglioside.

Alternatively, the “treatment targeting a cancer expressing the O-acetylated-GD2 ganglioside” may also be or comprise an immunoconjugate comprising such an antibody or an antigen-binding fragment recognizing, binding or targeting the O-acetylated-GD2 ganglioside.

The “treatment targeting a cancer expressing the O-acetylated-GD2 ganglioside” may also be or comprise a multimeric antibody or a multimeric antigen-binding fragment that recognizes, binds, or targets the O-acetylated-GD2 ganglioside.

The “treatment” may also comprise a chimeric antigen receptor (CAR) grafted onto an immune effector cell, such as e.g., a T cell, comprising an antigen-binding fragment of an antibody that binds to the O-acetylated-GD2 ganglioside.

A non-limiting example of antibody that binds to the O-acetylated-GD2 ganglioside is the mouse therapeutic antibody 8B6.

In some embodiments, the antibody that binds to the O-acetylated-GD2 ganglioside has a sequence comprising:
a light chain LC-CDR1 of sequence QSLLKNNGNTFL (SEQ ID NO: 37), an LC-CDR2 of sequence KVS, an LC-CDR3 of sequence SQSTHIPYT (SEQ ID NO: 38); and
a heavy chain HC-CDR1 of sequence EFTFTDYY (SEQ ID NO: 39), an HC-CDR2 of sequence IRNRANGYTT (SEQ ID NO: 40), an HC-CDR3 of sequence ARVSNWAFDY (SEQ ID NO: 41).

Preferably, the antibody that binds to the O-acetylated-GD2 ganglioside is a chimeric antibody, more preferably a humanized antibody or a human antibody.

In some embodiments, the antibody that binds to the O-acetylated-GD2 may be a humanized antibody derived from the mouse antibody 8B6.

In some embodiments, the antibody that binds to the O-acetylated-GD2 ganglioside a humanized antibody having a sequence comprising:
a light chain variable region (VL) of sequence SEQ ID NO: 42; and
a heavy chain variable region (VH) of sequence SEQ ID NO: 43.

In some embodiments, the antibody that binds to the O-acetylated-GD2 ganglioside a humanized antibody having a sequence comprising:
a light chain variable region (VL) of sequence SEQ ID NO: 51; and
a heavy chain variable region (VH) of sequence SEQ ID NO: 52.

Non-limiting examples of humanized antibodies that bind to the O-acetylated-GD2 ganglioside for instance include humanized antibodies having a sequence comprising:
a heavy chain variable region (VH) sequence selected from the group consisting of SEQ ID NO: 44 (“VH49A”), SEQ ID NO: 45 (“VH72A”), SEQ ID NO: 46 (“VH49BHS”), SEQ ID NO: 47 (“VH72BHNPS”); and
a light chain variable region (VL) sequence selected from the group consisting of SEQ ID NO: 48 (“VL30A”), SEQ ID NO: 49 (“VL28A”), SEQ ID NO: 50 (“VL28Bs01/A2”).

In some embodiments, the “treatment targeting a cancer expressing the O-acetylated-GD2 ganglioside” comprises an anti-cancer agent, optionally in combination with a therapy targeting the O-acetylated-GD2 ganglioside (such as e.g., an antibody, or an antigen-binding fragment, that binds to the O-acetylated-GD2 ganglioside, or a chimeric antigen receptor (CAR) grafted onto an immune effector cell (such as e.g., a T cell) comprising an antigen-binding fragment that binds to the O-acetylated-GD2 ganglioside).

Thus, the “treatment targeting a cancer expressing the O-acetylated-GD2 ganglioside” may comprises an anti-cancer agent, a therapy targeting the O-acetylated-GD2 ganglioside, or a combination thereof.

The term “anti-cancer agent” refers to a chemical, physical or biological agent or compound with anti-proliferative, anti-oncogenic and/or carcinostatic properties which can be used to inhibit cell or tumor growth, proliferation and/or development. Preferably, the anti-cancer agent has an activity against a cancer expressing the O-acetylated-GD2 ganglioside activity.

Examples of anti-cancer agents include, without limitation, platinum coordination compounds (such as, e.g., cisplatin, carboplatin or oxalyplatin); taxane compounds (such as, e.g., paclitaxel or docetaxel); topoisomerase I inhibitors (such as, e.g., irinotecan, topotecan or camptothecin); topoisomerase II inhibitors (such as, e.g., etoposide or teniposide); vinca alkaloids (such as, e.g., vinblastine, vincristine or vinorelbine); anti-tumor nucleoside derivatives (such as, e.g., 5-fluorouracil, gemcitabine or capecitabine); alkylating agents (such as, e.g., nitrogen mustard or nitrosourea, imidazotetrazines, cyclophosphamide, chlorambucil, carmustine or lomustine); anti-tumor anthracycline derivatives (such has, e.g., daunorubicin, doxorubicin, idarubicin or mitoxantrone); anti-HER2 antibodies (such as, e.g., trastuzumab); estrogen receptor antagonists or selective estrogen receptor modulators (such as, e.g., tamoxifen, toremifene, droloxifene, faslodex or raloxifene); aromatase inhibitors (such as, e.g., exemestane, anastrozole, letrazole or vorozole); differentiating agents (such as, e.g., retinoids, vitamin D and retinoic acid metabolism blocking agents [RAMBA] such as accutane); DNA methyl transferase inhibitors (such as, e.g., azacytidine); histone methyl transferase inhibitors (such as, e.g., GSK126); kinase inhibitors (such as, e.g., flavoperidol, imatinib mesylate or gefitinib); farnesyltransferase inhibitors; HDAC inhibitors; anti-tumor antibodies, mitotic inhibitors, tyrosine kinase inhibitors, corticosteroids, hormones or hormone-like drugs, cytokines, nucleic acids (such as, e.g., double-stranded synthetic short RNA molecules (miRNAs) or synthetic DNA/RNA-like oligonucleotides (ASOs)); anti-tumor antibiotics (such as, e.g., anthracyclines), prenol lipids, anti-metabolites (such as, e.g., diazines) and transition metal salts.

When the treatment is used to treat a cancer expressing the O-acetylated-GD2 ganglioside selected from the group comprising or consisting of neuroblastoma, glioma, retinoblastoma, Ewing's family of tumors, sarcoma (i.e., rhabdomyosarcoma, osteosarcoma, leiomyosarcoma, liposarcoma, and fibrosarcoma), small cell lung cancer, breast cancer, melanoma, metastatic renal carcinoma, head and neck cancer and hematological cancers (i.e., leukemia, Hodgkin lymphoma, non-Hodgkin lymphoma and myeloma), the anti-cancer agent is preferably selected from the group consisting of cyclophosphamide, doxorubicin, topotecan, irinotecan, temozolomide (TMZ), retinoic acid (RA), 5-Fluorouracil (5-FU), fludarabine, carboplatin, cisplatin, or a mixture thereof.

When the treatment is used to treat neuroblastoma, the anti-cancer agent is preferably temozolomide, topotecan, irinotecan, fludarabine, cyclophosphamide, or a mixture thereof.

In some embodiments, the drug treatment comprises at least an antibody that binds to the O-acetylated-GD2 ganglioside and an anti-cancer agent, that may be used in combination or may be formulated in a combination. Preferably, combining the antibody recognizing the O-acetylated-GD2 ganglioside and the anti-cancer agent gives rise to an unexpected technical effect, i.e., synergy.

The expression “combination” refers to any preparation comprising at least two components. The different components of the combination, may be used simultaneously, semi-simultaneously, separately, sequentially or spaced out over a period of time so as to obtain the maximum efficacy of the combination.

For instance, they may be administered concurrently, i.e., simultaneously in time, or sequentially, i.e., one component is administered after the other one(s). After administration of the first component, the other component(s) can be administered substantially immediately thereafter or after an effective time period. The effective time period is the amount of time given for realization of maximum benefit from the administration of the components.

As a result, a combination is not limited to one obtained by physical association of the constituents, but may also be in the form of separate products permitting a separate administration, which can be simultaneous or spaced out over a period of time. Alternatively, the different components may be co-formulated.

In some embodiments, the drug treatment is to be administered to the subject in need thereof in a therapeutically effective amount.

The term “therapeutically effective amount”, as used herein, refers to an amount effective, at dosages and for periods of time necessary, to achieve a desired preventive and/or therapeutic result.

In the context of the invention, the term “therapeutically effective amount” is intended to encompass any amount that will achieve the desired therapeutic or biological effect. The therapeutic effect is dependent upon the cancer expressing the O-acetylated-GD2 ganglioside treated or the biological effect desired. As such, the therapeutic effect can be a decrease in the severity of symptoms associated with the cancer expressing the O-acetylated-GD2 ganglioside, and/or inhibition (partial or complete) of the progression of the cancer expressing the O-acetylated-GD2 ganglioside. The amount needed to elicit the therapeutic response can be determined based on the cancer type, the age, health, size and sex of the patient. Doses can be adjusted to the size of other mammals, in accordance with weight or square meter size.

It will be however understood that the total daily usage of the treatment will be decided by the attending physician within the scope of sound medical judgment. The specific therapeutically effective dose level for any particular patient will depend upon a variety of factors including the disease being treated and the severity of the disease; activity of the treatment employed; the age, body weight, general health, sex and diet of the subject; the time of administration, route of administration, and rate of excretion of the treatment employed; the duration of the treatment; and so on. For example, it is well within the skill of the art to start doses of the compound at levels lower than those required to achieve the desired therapeutic effect and to gradually increase the dosage until the desired effect is achieved.

The total dose required for each treatment may be administered by multiple doses or in a single dose.

Another aspect of the invention pertains to an inhibitor of CASD1 for use in the treatment of a cancer expressing the O-acetylated-GD2 ganglioside.

Also, the inventors surprisingly showed that the silencing of certain genes, or the inhibition of corresponding proteins, namely CERK, PIK3C2A, PDK3, MERTK NME3 and EZH2, resulted in an upregulation of O-acetylated-GD2 ganglioside expression.

The O-acetylated-GD2 ganglioside being a first-class target antigen for cancer immunotherapy, it may be desirable to upregulate O-acetylated-GD2 ganglioside expression while targeting said O-acetylated-GD2 ganglioside with a suitable therapy.

Therefore, the invention also concerns an inhibitor of a biomarker selected from the group consisting of CERK, PIK3C2A, PDK3, MERTK, NME3 and EZH2, in combination with a therapy targeting the O-acetylated-GD2 ganglioside, for use in the treatment of a cancer expressing the O-acetylated-GD2 ganglioside.

In particular, the “therapy targeting the O-acetylated-GD2 ganglioside” may be an immunotherapy, such as e.g., an antibody, or an antigen-binding fragment, that binds to the O-acetylated-GD2 ganglioside, or a chimeric antigen receptor (CAR) grafted onto an immune effector cell (such as e.g., a T cell) comprising an antigen-binding fragment that binds to the O-acetylated-GD2 ganglioside.

By “inhibitor” of a protein, it is meant a compound that inhibits the expression and/or the activity of said protein. Preferably, the inhibitors according to the invention are direct inhibitors that bind to their target genes, nucleic acids or proteins.

The inhibitor of a protein selected among CASD1, CERK, PIK3C2A, PDK3, MERTK, NME3 or EZH2 may be a small molecule, a chemical, an organic or inorganic compound, a peptide inhibitor, a peptidomimetic, an antibody or an antigen-binding fragment, a lipid, an antisense oligonucleotide targeting the gene, an interfering RNA directed against the mRNA or the pre-mRNA of said protein, an aptamer, or a ribozyme directed against the mRNA or the pre-mRNA of said protein.

The inhibitors of the expression and/or the activity of a protein are capable of reducing the expression of said protein, or the activity of said protein, by at least 10%, preferably by 30%, more preferably by at least 50%, and advantageously by at least 70%, 75%, 80%, 85%, 90%, 95%, or 100%.

The term “small molecule” or “small molecule inhibitor” refers to a molecule of less than 1,000 daltons in size, in particular of organic or inorganic compounds.

When the inhibitor is an antibody or an antigen-binding fragment, it preferably inhibits the activity of its target protein. The antibody or antigen-binding fragment may be a chimeric antibody, more preferably a humanized antibody or a human antibody. The term “humanized antibody” refers to an antibody produced in a non-human animal, which maintains its binding specificity to its target protein, but in which most non-human sequences have been replaced by the corresponding human sequences, in order to reduce its immunogenicity in human.

Antibody-binding fragments of an antibody include, without any limitation, single chain antibodies, Fv, dsFv, Fab, Fab′, Fab′-SH, F(ab)′2, scFv, Fd, VHH, defucosylated antibodies, diabodies, triabodies and tetrabodies.

The term “interfering RNA” refers to a double stranded RNA molecule capable of inhibiting in a sequence specific manner the expression of a target gene by causing the degradation of the mRNA thereof.

In order to be used in the mammalian cell, the interfering RNAs must possess a double stranded portion of less than 30 bp (base pairs) in order to avoid a non-specific interferon response induced by longer double stranded RNAs. These interfering RNAs, which are RNAs containing both the target sequence as well as the corresponding antisense sequence, include in particular the small interfering RNAs (“small interfering RNAs” or siRNAs), the short RNAs having the shape of a hairpin (“short hairpin RNAs” or shRNAs), which are then transformed by the cellular machinery into siRNAs, as well as the pre-miRNAs and miRNAs.

“Aptamers” are a class of molecule which represents an alternative to antibodies in term of molecular recognition. Aptamers are oligonucleotide or oligopeptide sequences that have the ability to recognize practically all categories of target molecules with a high affinity and high specificity.

The term “ribozyme” refers to an RNA molecule having an enzymatic activity that is capable of cleaving other distinct RNA molecules, with the cleavage being specific to a given target ribonucleotide sequence. In the present case, the target ribonucleotide sequence is included in the sequence of the mRNA or the pre-mRNA derived from the transcription of the gene encoding CASD1, CERK, PIK3C2A, PDK3, MERTK, NME3 or EZH2.

Non-limiting examples of CASD1 inhibitors include e.g., the C7orf12 anti-CASD1 rabbit polyclonal antibody.

Non-limiting examples of CERK inhibitors include e.g., the rabbit anti-CERK polyclonal antibody (SAB2701099, Sigma Aldrich), the rabbit anti-human Ceramide Kinase/CERK polyclonal antibody (LS-A4556, LSBio), or the small molecule NVP-231 (a reversible ceramide kinase inhibitor that competitively inhibits binding of ceramide to CERK).

Non-limiting examples of PI3KC2A inhibitors include e.g., the mouse anti-PIK3C2A monoclonal antibody Clone OTI2C2 (MA5-26506, Invitrogen), the mouse anti-human PIK3C2A monoclonal antibody Clone 3E7 (LS-B6117, LSBio), or the small molecule PIK-90 (PI 3-K inhibitor IX).

Non-limiting examples of PDK3 inhibitors include e.g., the rabbit anti-PDK3 polyclonal antibody (PA5-76332, Invitrogen), the rabbit anti-PDK3 polyclonal antibody (ab154549, Abcam), or the rabbit anti-PDK3 polyclonal antibody (HPA046583, Atlas antibodies), or the small molecule inhibitor Quercetin.

Non-limiting examples of MERTK inhibitors include e.g., the rabbit recombinant anti-MERTK monoclonal antibody Clone Y323 (ab52968, Abcam), or the small molecule inhibitor UNC1666.

Non-limiting examples of NME3 inhibitors include e.g., the rabbit recombinant anti-NME3 antibody Clone EPR13117 (ab181257, Abcam), or the rabbit anti-human NME3 polyclonal antibody (epitope aa51-81) (LS-C328074, LSBio).

Non-limiting examples of EZH2 inhibitors include e.g., the methyltransferase inhibitor GSK126 (GSK2816126A, GSK2816126).

The following items are also herein disclosed:

Item 1: An in vitro method of selecting a subject suffering from a cancer expressing the O-acetylated-GD2 ganglioside for treatment targeting said cancer, said method comprising the steps of:
a1) measuring CASD1 expression level as a biomarker in a biological sample of the subject; and
b) based on the level measured at step a1), selecting said subject to undergo treatment targeting said cancer expressing the O-acetylated-GD2 ganglioside.

Item 2: An in vitro method of monitoring the response of a subject suffering from a cancer expressing the O-acetylated-GD2 ganglioside to a treatment targeting said cancer, said method comprising the steps of:
a1) measuring CASD1 expression level as a biomarker in a biological sample of the subject; and
b) based on the level measured at step a1), monitoring the response of said subject to said treatment.

Item 3: An in vitro method of diagnosing a cancer expressing the O-acetylated-GD2 ganglioside in a subject, said method comprising the steps of:
a1) measuring CASD1 expression level as a biomarker in a biological sample of the subject; and
b) based on the level measured at step a1), diagnosing a cancer expressing the O-acetylated-GD2 ganglioside in said subject.

Item 4: The in vitro method according to any one of items 1 to 3, further comprising a step a2) of comparing the expression level measured at step a1) with a threshold value.

Item 5: The in vitro method according to item 4, wherein the subject is selected to undergo a treatment targeting said cancer expressing the O-acetylated-GD2 ganglioside, or is diagnosed as suffering from a cancer expressing the O-acetylated-GD2 ganglioside, if the expression level measured at step a1) is higher than the threshold value.

Item 6: The in vitro method according to any one of items 1 to 5, wherein the biological sample is selected from the group consisting of a blood sample, a serum sample, a plasma sample, a urine sample, a tissue sample from a biopsy and a cell sample from a biopsy.

Item 7: The in vitro method according to any one of items 1 to 6, wherein CASD1 expression level measured at step a1) is measured at the DNA or RNA level, preferably by RT-PCR, RT-qPCR, Northern Blot, hybridization techniques, microarrays or sequencing.

Item 8: The in vitro method according to any one of items 1 to 6, wherein CASD1 expression level measured at step a1) is measured at the protein level, preferably by FACS, immunohistochemistry, mass spectrometry, western blot associated with cell fractionation, enzyme-linked immunosorbent assay (ELISA), sandwich ELISA, fluorescent-linked immunosorbent assay (FLISA), enzyme immunoassay (EIA), radioimmunoassay (RIA) or image analysis.

Item 9: The in vitro method according to any one of items 1 to 8, said method further comprising the steps of:
a1′) measuring the expression level of at least one biomarker selected from the group consisting of OAcGD2, GD2, GD2S, GD3S, CERK, PIK3C2A, PDK3, MERTK and NME3, in a biological sample of the subject; and
b′) based on the levels measured at step a1) and step a1′), selecting said subject to undergo a treatment targeting said cancer expressing the O-acetylated-GD2 ganglioside, monitoring the response of said subject to said treatment, or diagnosing a cancer expressing the O-acetylated-GD2 ganglioside in said subject.

Item 10: The in vitro method according to any one of items 1-2 or 9, wherein said treatment comprises an antibody that binds to the O-acetylated-GD2 ganglioside.

Item 11: Use of CASD1 as a biomarker of a cancer expressing the O-acetylated-GD2 ganglioside.

Item 12: The in vitro method according to any one of items 1 to 10, or the use according to item 11, wherein said cancer expressing the O-acetylated-GD2 ganglioside is characterized by the presence of cells expressing the O-acetylated-GD2 ganglioside at their cell surface in the subject.

Item 13: The in vitro method according to any one of items 1 to 10, or the use according to item 11, wherein said cancer expressing the O-acetylated-GD2 ganglioside is selected from the group consisting of neuroblastoma, glioma (including glioblastoma), retinoblastoma, Ewing's family of tumors, sarcoma (including rhabdomyosarcoma, osteosarcoma, leiomyosarcoma, liposarcoma, and fibrosarcoma), lung cancer (including small cell lung cancer), breast cancer, melanoma (including uveal melanoma), metastatic renal carcinoma, head and neck cancer, hematological cancers (including leukemia, Hodgkin lymphoma, non-Hodgkin lymphoma and myeloma), colorectal cancer, pancreatic cancer, prostate cancer, liver cancer, bladder cancer, gastric cancer, cervical cancer, endometrial cancer, neuroendocrine cancer, esophageal cancer, ovarian cancer, skin cancer, kidney cancer, soft tissue sarcoma, adrenal cancer, testicular cancer and thyroid cancer.

Item 14: An inhibitor of CASD1 for use in the treatment of a cancer expressing the O-acetylated-GD2 ganglioside.

Item 15: An inhibitor of a biomarker selected from the group consisting of CERK, PIK3C2A, PDK3, MERTK and NME3, in combination with an inhibitor of the O-acetylated-GD2 ganglioside, for use in the treatment of a cancer expressing the O-acetylated-GD2 ganglioside.

### BRIEF DESCRIPTION OF THE DRAWINGS

FIG. 1 corresponds to photographs of Western-blot showing that CASD1 induces the O-acetylation of GD2 and GD3 in CHO cells. Total gangliosides were extracted from CHO-WT or CHOΔCasd1 cells transfected with empty vector (mock) or a plasmid encoding the indicated synthases. Gangliosides were separated by thin-layer chromatography and stained with the indicated antibodies. Pure gangliosides and their in vitro generated 9-O-acetylated forms were used as standards (left panel). Please note that the OAcGD2 standard contains residual amounts of GD2. FIG. 1A: CASD1-dependent formation of 9-OAcGD2. FIG. 1B: CASD1-dependent formation of 9-O-Ac-GD3.

FIG. 2 is a graph showing CASD1 expression in neuro-ectoderm derived cancer cells. CASD1 mRNA expression was determined by qPCR in breast cancer cell lines. SK-MEL-28 melanoma cell line and LAN-1 neuroblastoma cell line were used as controls. Results were normalized to the expression of HPRT (hypoxanthine phosphoribosyl transferase) mRNA. Each bar represents the mean±SD of n=3 experiments.

FIG. 3 is a set of graphs showing the reduced OAcGD2 expression in SUM159PT cells depleted for CASD1 expression using siRNA strategy. qPCR quantification of GD2S (FIG. 3A) and CASD1 (FIG. 3B) expression in transiently transfected and control SUM159PT cells (n=3). Results were normalized to the expression of HPRT mRNA. Quantification of the mean fluorescence intensity of GD2 (FIG. 3C) and OAcGD2 (FIG. 3D) expression using immunocytochemistry and confocal microscopy in SUM159PT cells (n=3). Statistical difference using unpaired t-test: *p<0.5; ****p<0.0001; ns: not significant.

FIG. 4 is a set of graphs showing increased OAcGD2 expression in CASD1 overexpressing SUM159PT cells using plasmid transfection (CADS1+). qPCR quantification of GD2S (FIG. 4A) and CASD1 (FIG. 4B) genes in transiently transfected SUM159PT cells (n=3). Results were normalized to the expression of HPRT mRNA. Quantification of mean fluorescence intensity of GD2 (FIG. 4C) and OAcGD2 (FIG. 4D) expression by immunochemistry and confocal microscopy in breast cancer cells (n=3). Statistical difference using unpaired t-test: **** p<0.0001; ns: non-significant.

FIG. 5 is a set of graphs showing expression of CASD1 mRNA and quantification of OAcGD2 and GD2 expression in SUM159PT CASD1+clones. FIG. 5A is a graph showing RT-qPCR quantification of CASD1 gene expression in stably transfected and control SUM159PT cells (n=3). Results were normalized to HPRT mRNA expression. FIG. 5B is a graph representing the quantification of mean fluorescence intensity of GD2. Statistical difference using unpaired t-test: **** p<0.0001. FIG. 5C is a graph representing the quantification of mean fluorescence intensity of OAcGD2. Statistical difference using unpaired t-test: **** p<0.0001.

FIG. 6 is a set of graphs demonstrating the biological properties of SUM159PT CASD1+clones. The growth of control and SUM159PT CASD1+#19 and #26 clones was assessed after 0 h, 24 h, 48 h, 72 h and 96 h using MTS reagent (Promega) in media containing 5% (FIG. 6A), 1% (FIG. 6B) or 0% (FIG. 6C) of fetal calf serum (FCS). The migration (FIG. 6D) and invasion (FIG. 6E) capabilities of control and SUM159PT CASD1+clones #19 and #26 were assessed after 48 h by Transwell assay in serum free media. Statistical difference using one-way anova: **** p<0.0001; ** p<0.002; * p<0.02.

FIG. 7 is a set of chemical structures representing GD2 and two illustrative examples of OAcGD2 gangliosides. FIG. 7A represents the chemical structure of GD2 (Neu5Acα2-8Neu5Acα2-3[GalNAcβ1-4]LacCer). FIG. 7B represents the chemical structure of a first example of a 9-O-acetylated GD2 isomer (Neu5,9Ac₂α2-8Neu5Acα2-3[GalNAcβ1-4]LacCer). FIG. 7C represents the chemical structure of a second example of a 9-O-acetylated GD2 isomer (Neu5Acα2-8Neu5,9Ac₂α2-3[GalNAcβ1-4]LacCer). FIG. 7B and 7C are examples of possible GD2 O-acetylation isomers. However, GD2 may also be O-acetylated at other positions within the molecule.

FIG. 8A-O is a set of forest plots showing the hazard ratio and 95% confidence intervals in patients having high and low expression levels of CASD1, CERK, PIK3C2A, B4GALTN1, ST8SIA1 genes and combinations thereof, in TCGA cohorts. Hazard ratio was calculated in populations computationally identified as having high or low expression of the gene of interest, based on individual signature expression in TCGA datasets by SurvExpress and tcgasurvival optimized algorithm. The datasets analyzed were Sarcoma (SARC); Pheochromocytoma and Paraganglioma (PCPG); Uterine Corpus Endometrial Carcinoma (UCEC); Thyroid carcinoma (THCA); Thymoma (THYM); Testicular Germ Cell Tumors (TGCT); Stomach adenocarcinoma (STAD); Skin Cutaneous Melanoma (SKCM); Prostate adenocarcinoma (PRAD); Pancreatic adenocarcinoma (PAAD); Ovarian serous cystadenocarcinoma (OV); Lung squamous cell carcinoma (LUSC); Lung adenocarcinoma (LUAD); Liver hepatocellular carcinoma (LIHC); Kidney PAN cancer (KIPAN); Acute Myeloid Leukemia (LAML); Head and Neck squamous cell carcinoma (HNSC); Uveal Melanoma (UVM); Esophageal carcinoma (ESCA); Colon and Rectum adenocarcinoma (COADREAD); Cervical squamous cell carcinoma and endocervical adenocarcinoma (CESC); Breast invasive carcinoma (BRCA); Gliomas (GBM and LGG); Bladder Urothelial Carcinoma (BLCA); Cholangiocarcinoma (CHOL); Adrenocortical carcinoma (ACC). P-value is indicated on the graph (*** p<0.001, ** p<0.01, * p<0.05). Statistically significant hazard ratios are indicated in black, while non-significant hazard ratios are in dark grey. FIG. 8A represents B4GALNT1, FIG. 8B represents ST8SIA, FIG. 8C represents CASD1, FIG. 8D represents CASD1/B4GALNT1, FIG. 8E represents CASD1/ST8SIA, FIG. 8F represents CASD1/B4GALNT1/ST8SIA, FIG. 8G represents CERK, FIG. 8H represents CERK/B4GALNT1, FIG. 8I represents CERK/ST8SIA, FIG. 8J represents CERK/B4GALNT1/ST8SIA, FIG. 8K represents PIK3C2A, FIG. 8L represents PIK3C2A/B4GALNT1, FIG. 8M represents PIK3C2A/ST8SIA, FIG. 8N represents PIK3C2A/B4GALNT1/ST8SIA, FIG. 8O represents CASD1, FIG. 8P represents CASD1/CERK, FIG. 8Q represents CASD1/PIK3C2A.

FIG. 9 is a bar plot showing the quantification of the mean fluorescence intensity of O-acetylated GD2 ganglioside in breast cancer cell line MDA-MB-231 GD3S+treated with a CERK inhibitor for 2, 4, 6, 8, 20, 24 or 48 hours and stained by immunocytochemistry with an anti-OAcGD2 antibody. Mean fluorescence intensity was measured by confocal microscopy.

FIG. 10 is a bar plot showing the migration capacity of the breast cancer cell line MDA-MB-231 GD3S+treated with a CERK inhibitor. Cells were placed in a Transwell in presence or absence of CERK inhibitor and counted after 24 hours of treatment.

FIG. 11 is a bar plot showing the quantitative real-time PCR quantification of CERK mRNA expression in transiently transfected breast cancer cell line MDA-MB231 GD3S+. MDA-MB-231 GD3S+cells were transfected with either a control siRNA (siControl), or a siRNA targeting CERK (siCERK2 or siCERK4). Results were normalized to the expression of HPRT mRNA.

FIG. 12A-D are representative confocal microscopy photographs of the analysis of OAcGD2 expression in transiently transfected breast cancer cell line MDA-MB-231 GD3S+. Cells were stained by immunocytochemistry using an anti-OAcGD2 antibody. FIG. 12A shows cells transfected with a control siRNA (siControl). FIG. 12B shows cells transfected with a siRNA targeting CASD1 (siCASD1). FIG. 12C shows cells transfected with a siRNA targeting CERK (siCERK2). FIG. 12D shows cells transfected with a siRNA targeting CERK (siCERK4).

FIG. 13 is a bar plot showing the quantification of the mean fluorescence intensity of O-acetylated GD2 ganglioside in transiently transfected breast cancer cell line MDA-MB-231 GD3S+. MDA-MB-231 GD3S+cells were transiently transfected either with a control siRNA (siControl), a siRNA targeting CASD1 (siCASD1), or one of two different siRNA targeting CERK (siCERK2 or siCERK4). Cells were stained by immunocytochemistry using an anti-OAcGD2 antibody. Mean fluorescence intensity was quantified by confocal microscopy.

FIG. 14 is a bar plot showing the migration capacity of transiently transfected breast cancer cell line MDA-MB-231 GD3S+. MDA-MB-231 GD3S+cells were transiently transfected either with a control siRNA (siControl), a siRNA targeting CASD1 (siCASD1), or one of two different siRNA targeting CERK (siCERK2 or siCERK4). Cells were placed in a Transwell and counted after 24 hours of treatment.

TABLE OF SEQUENCES

| SEQ | | |
|---|---|---|
| ID | | |
| NO | Sequence function | Sequence |
| 1 | CASD1 human protein | MAALAYNLGKREINHYFSVRSAKVLALVAVLLLAACHLASRRYRGNDS |
| | | CEYLLSSGRFLGEKVWQPHSCMMHKYKISEAKNCLVDKHIAFIGDSRIRQ |
| | | LFYSFVKIINPQFKEEGNKHENIPFEDKTASVKVDFLWHPEVNGSMKQCI |
| | | KVWTEDSIAKPHVIVAGAATWSIKIHNGSSEALSQYKMNITSIAPLLEKLA |
| | | KTSDVYWVLQDPVYEDLLSENRKMITNEKIDAYNEAAVSILNSSTRNSKS |
| | | NVKMFSVSKLIAQETIMESLDGLHLPESSRETTAMILMNVYCNKILKPVD |
| | | GSCCQPRPPVTLIQKLAACFFTLSIIGYLIFYIIHRNAHRKNKPCTDLESGEE |
| | | KKNIINTPVSSLEILLQSFCKLGLIMAYFYMCDRANLFMKENKFYTHSSFF |
| | | IPIIYILVLGVFYNENTKETKVLNREQTDEWKGWMQLVILIYHISGASTFL |
| | | PVYMHIRVLVAAYLFQTGYGHFSYFWIKGDFGIYRVCQVLFRLNFLVVV |
| | | LCIVMDRPYQFYYFVPLVTVWFMVIYVTLALWPQIIQKKANGNCFWHFG |
| | | LLLKLGFLLLFICFLAYSQGAFEKIFSLWPLSKCFELKGNVYEWWFRWRL |
| | | DRYVVFHGMLFAFIYLALQKRQILSEGKGEPLFSNKISNFLLFISVVSFLTY |
| | | SIWASSCKNKAECNELHPSVSVVQILAFILIRNIPGYARSVYSSFFAWFGKI |
| | | SLELFICQYHIWLAADTRGILVLIPGNPMLNIIVSTFIFVCVAHEISQITNDL |
| | | AQIIIPKDNSSLLKRLACIAAFFCGLLILSSIQDKSKH |
| 2 | GD2 synthase human | MWLGRRALCALVLLLACASLGLLYASTRDAPGLRLPLAPWAPPQSPRRP |
| | protein | ELPDLAPEPRYAHIPVRIKEQVVGLLAWNNCSCESSGGGLPLPFQKQVRA |
| | | IDLTKAFDPAELRAASATREQEFQAFLSRSQSPADQLLIAPANSPLQYPLQ |
| | | GVEVQPLRSILVPGLSLQAASGQEVYQVNLTASLGTWDVAGEVTGVTLT |
| | | GEGQADLTLVSPGLDQLNRQLQLVTYSSRSYQTNTADTVRFSTEGHEAA |
| | | FTIRIRHPPNPRLYPPGSLPQGAQYNISALVTIATKTFLRYDRLRALITSIRR |
| | | FYPTVTVVIADDSDKPERVSGPYVEHYLMPFGKGWFAGRNLAVSQVTTK |
| | | YVLWVDDDFVFTARTRLERLVDVLERTPLDLVGGAVREISGFATTYRQL |
| | | LSVEPGAPGLGNCLRQRRGFHHELVGFPGCVVTDGVVNFFLARTDKVRE |
| | | VGFDPRLSRVAHLEFFLDGLGSLRVGSCSDVVVDHASKLKLPWTSRDAG |
| | | AETYARYRYPGSLDESQMAKHRLLFFKHRLQCMTSQ |
| 3 | GD3 synthase human | MSPCGRARRQTSRGAMAVLAWKFPRTRLPMGASALCVVVLCWLYIFPV |
| | protein | YRLPNEKEIVQGVLQQGTAWRRNQTAARAFRKQMEDCCDPAHLFAMT |
| | | KMNSPMGKSMWYDGEFLYSFTIDNSTYSLFPQATPFQLPLKKCAVVGNG |
| | | GILKKSGCGRQIDEANFVMRCNLPPLSSEYTKDVGSKSQLVTANPSIIRQR |
| | | FQNLLWSRKTFVDNMKIYNHSYIYMPAFSMKTGTEPSLRVYYTLSDVGA |
| | | NQTVLFANPNFLRSIGKFWKSRGIHAKRLSTGLFLVSAALGLCEEVAIYG |
| | | FWPFSVNMHEQPISHHYYDNVLPFSGFHAMPEEFLQLWYLHKIGALRMQ |
| | | LDPCEDTSLQPTS |
| 4 | CERK human protein | MGATGAAEPLQSVLWVKQQRCAVSLEPARALLRWWRSPGPGAGAPGA |
| | | DACSVPVSEIIAVEETDVHGKHQGSGKWQKMEKPYAFTVHCVKRARRH |
| | | RWKWAQVTFWCPEEQLCHLWLQTLREMLEKLTSRPKHLLVFINPFGGK |
| | | GQGKRIYERKVAPLFTLASITTDIIVTEHANQAKETLYEINIDKYDGIVCV |
| | | GGDGMFSEVLHGLIGRTQRSAGVDQNHPRAVLVPSSLRIGIIPAGSTDCV |
| | | CYSTVGTSDAETSALHIVVGDSLAMDVSSVHHNSTLLRYSVSLLGYGFY |
| | | GDIIKDSEKKRWLGLARYDFSGLKTFLSHHCYEGTVSFLPAQHTVGSPRD |
| | | RKPCRAGCFVCRQSKQQLEEEQKKALYGLEAAEDVEEWQVVCGKFLAI |
| | | NATNMSCACRRSPRGLSPAAHLGDGSSDLILIRKCSRFNFLRFLIRHTNQQ |
| | | DQFDFTFVEVYRVKKFQFTSKHMEDEDSDLKEGGKKRFGHICSSHPSCC |
| | | CTVSNSSWNCDGEVLHSPAIEVRVHCQLVRLFARGIEENPKPDSHS |
| 5 | PIK3C2A human | MAQISSNSGFKECPSSHPEPTRAKDVDKEEALQMEAEALAKLQKDRQVT |
| | protein | DNQRGFELSSSTRKKAQVYNKQDYDLMVFPESDSQKRALDIDVEKLTQA |
| | | ELEKLLLDDSFETKKTPVLPVTPILSPSFSAQLYFRPTIQRGQWPPGLPGPS |
| | | TYALPSIYPSTYSKQAAFQNGFNPRMPTFPSTEPIYLSLPGQSPYFSYPLTP |
| | | ATPFHPQGSLPIYRPVVSTDMAKLFDKIASTSEFLKNGKARTDLEITDSKV |
| | | SNLQVSPKSEDISKFDWLDLDPLSKPKVDNVEVLDHEEEKNVSSLLAKDP |
| | | WDAVLLEERSTANCHLERKVNGKSLSVATVTRSQSLNIRTTQLAKAQGH |
| | | ISQKDPNGTSSLPTGSSLLQEVEVQNEEMAAFCRSITKLKTKFPYTNHRTN |
| | | PGYLLSPVTAQRNICGENASVKVSIDIEGFQLPVTFTCDVSSTVEIIIMQAL |
| | | CWVHDDLNQVDVGSYVLKVCGQEEVLQNNHCLGSHEHIQNCRKWDTEI |
| | | RLQLLTFSAMCQNLARTAEDDETPVDLNKHLYQIEKPCKEAMTRHPVEE |
| | | LLDSYHNQVELALQIENQHRAVDQVIKAVRKICSALDGVETLAITESVKK |
| | | LKRAVNLPRSKTADVTSLFGGEDTSRSSTRGSLNPENPVQVSINQLTAAIY |
| | | DLLRLHANSGRSPTDCAQSSKSVKEAWTTTEQLQFTIFAAHGISSNWVSN |
| | | YEKYYLICSLSHNGKDLFKPIQSKKVGTYKNFFYLIKWDELIIFPIQISQLP |
| | | LESVLHLTLFGILNQSSGSSPDSNKQRKGPEALGKVSLPLFDFKRFLTCGT |
| | | KLLYLWTSSHTNSVPGTVTKKGYVMERIVLQVDFPSPAFDIIYTTPQVDR |
| | | SIIQQHNLETLENDIKGKLLDILHKDSSLGLSKEDKAFLWEKRYYCFKHP |
| | | NCLPKILASAPNWKWVNLAKTYSLLHQWPALYPLIALELLDSKFADQEV |
| | | RSLAVTWIEAISDDELTDLLPQFVQALKYEIYLNSSLVQFLLSRALGNIQIA |
| | | HNLYWLLKDALHDVQFSTRYEHVLGALLSVGGKRLREELLKQTKLVQL |
| | | LGGVAEKVRQASGSARQVVLQRSMERVQSFFQKNKCRLPLKPSLVAKEL |
| | | NIKSCSFFSSNAVPLKVTMVNADPMGEEINVMFKVGEDLRQDMLALQMI |
| | | KIMDKIWLKEGLDLRMVIFKCLSTGRDRGMVELVPASDTLRKIQVEYGV |
| | | TGSFKDKPLAEWLRKYNPSEEEYEKASENFIYSCAGCCVATYVLGICDRH |
| | | NDNIMLRSTGHMFHIDFGKFLGHAQMFGSFKRDRAPFVLTSDMAYVING |
| | | GEKPTIRFQLFVDLCCQAYNLIRKQTNLFLNLLSLMIPSGLPELTSIQDLKY |
| | | VRDALQPQTTDAEATIFFTRLIESSLGSIATKFNFFIHNLAQLRFSGLPSND |
| | | EPILSFSPKTYSFRQDGRIKEVSVFTYHKKYNPDKHYIYVVRILREGQIEPS |
| | | FVFRTFDEFQELHNKLSIIFPLWKLPGFPNRMVLGRTHIKDVAAKRKIELN |
| | | SYLQSLMNASTDVAECDLVCTFFHPLLRDEKAEGIARSADAGSFSPTPGQ |
| | | IGGAVKLSISYRNGTLFIMVMHIKDLVTEDGADPNPYVKTYLLPDNHKTS |
| | | KRKTKISRKTRNPTFNEMLVYSGYSKETLRQRELQLSVLSAESLRENFFL |
| | | GGVTLPLKDFNLSKETVKWYQLTAATYL |
| 6 | PDK3 human protein | MRLFRWLLKQPVPKQIERYSRFSPSPLSIKQFLDFGRDNACEKTSYMFLR |
| | | KELPVRLANTMREVNLLPDNLLNRPSVGLVQSWYMQSFLELLEYENKSP |
| | | EDPQVLDNFLQVLIKVRNRHNDVVPTMAQGVIEYKEKFGFDPFISTNIQY |
| | | FLDRFYTNRISFRMLINQHTLLFGGDTNPVHPKHIGSIDPTCNVADVVKD |
| | | AYETAKMLCEQYYLVAPELEVEEFNAKAPDKPIQVVYVPSHLFHMLFEL |
| | | FKNSMRATVELYEDRKEGYPAVKTLVTLGKEDLSIKISDLGGGVPLRKID |
| | | RLFNYMYSTAPRPSLEPTRAAPLAGFGYGLPISRLYARYFQGDLKLYSME |
| | | GVGTDAVIYLKALSSESFERLPVFNKSAWRHYKTTPEADDWSNPSSEPRD |
| | | ASKYKAKQ |
| 7 | MERTK human | MGPAPLPLLLGLFLPALWRRAITEAREEAKPYPLFPGPFPGSLQTDHTPLL |
| | protein | SLPHASGYQPALMFSPTQPGRPHTGNVAIPQVTSVESKPLPPLAFKHTVG |
| | | HIILSEHKGVKFNCSISVPNIYQDTTISWWKDGKELLGAHHAITQFYPDDE |
| | | VTAIIASFSITSVQRSDNGSYICKMKINNEEIVSDPIYIEVQGLPHFTKQPES |
| | | MNVTRNTAFNLTCQAVGPPEPVNIFWVQNSSRVNEQPEKSPSVLTVPGLT |
| | | EMAVFSCEAHNDKGLTVSKGVQINIKAIPSPPTEVSIRNSTAHSILISWVPG |
| | | FDGYSPFRNCSIQVKEADPLSNGSVMIFNTSALPHLYQIKQLQALANYSIG |
| | | VSCMNEIGWSAVSPWILASTTEGAPSVAPLNVTVFLNESSDNVDIRWMK |
| | | PPTKQQDGELVGYRISHVWQSAGISKELLEEVGQNGSRARISVQVHNAT |
| | | CTVRIAAVTRGGVGPFSDPVKIFIPAHGWVDYAPSSTPAPGNADPVLIIFG |
| | | CFCGFILIGLILYISLAIRKRVQETKFGNAFTEEDSELVVNYIAKKSFCRRAI |
| | | ELTLHSLGVSEELQNKLEDVVIDRNLLILGKILGEGEFGSVMEGNLKQED |
| | | GTSLKVAVKTMKLDNSSQREIEEFLSEAACMKDFSHPNVIRLLGVCIEMS |
| | | SQGIPKPMVILPFMKYGDLHTYLLYSRLETGPKHIPLQTLLKFMVDIALG |
| | | MEYLSNRNFLHRDLAARNCMLRDDMTVCVADFGLSKKIYSGDYYRQGR |
| | | IAKMPVKWIAIESLADRVYTSKSDVWAFGVTMWEIATRGMTPYPGVQN |
| | | HEMYDYLLHGHRLKQPEDCLDELYEIMYSCWRTDPLDRPTFSVLRLQLE |
| | | KLLESLPDVRNQADVIYVNTQLLESSEGLAQGSTLAPLDLNIDPDSIIASCT |
| | | PRAAISVVTAEVHDSKPHEGRYILNGGSEEWEDLTSAPSAAVTAEKNSVL |
| | | PGERLVRNGVSWSHSSMLPLGSSLPDELLFADDSSEGSEVLM |
| 8 | NME3 human protein | MICLVLTIFANLFPAACTGAHERTFLAVKPDGVQRRLVGEIVRRFERKGF |
| | | KLVALKLVQASEELLREHYAELRERPFYGRLVKYMASGPVVAMVWQGL |
| | | DVVRTSRALIGATNPADAPPGTIRGDFCIEVGKNLIHGSDSVESARREIAL |
| | | WFRADELLCWEDSAGHWLYE |
| 9 | CASD1 PCR primer | GCTCGGGATCCGCGGCTCTGGCCTACAACCTG |
| 10 | CASD1 PCR primer | GCTCGCTCGAGATGTTTTGATTTATCTTGAATGGATG |
| 11 | V5 epitope pre- | AGCTTCGAATGGGTAAGCCTATCCCTAACCCTCTCCTCGGTCTCGATT |
| | hybridized | CTACGG |
| | oligonucleotide | |
| 12 | V5 epitope pre- | GATCCCGTAGAATCGAGACCGAGGAGAGGGTTAGGGATAGGCTTACC |
| | hybridized | CATTCGA |
| | oligonucleotide | |
| 13 | Myc epitope pre- | TCGAGGAACAAAAACTCATCTCAGAAGAGGATCTGAATTAAT |
| | hybridized | |
| | oligonucleotide | |
| 14 | Myc epitope pre- | CTAGATTAATTCAGATCCTCTTCTGAGATGAGTTTTTGTTCC |
| | hybridized | |
| | oligonucleotide | |
| 15 | S94A mutagenesis | GCATTTATTGGAGATGCCAGAATTCGTCAATTG |
| | primer | |
| 16 | S94A mutagenesis | CAATTGACGAATTCTGGCATCTCCAATAAATGC |
| | primer | |
| 17 | ST8SIA1 PCR primer | GCTAAGCTTCGAATGGGTAAGCCTATCCCTAACCCTCTCCTCGGTCTC |
| | (G3DS) | GATTCTACGGGTACCAGCCCCTGCGGGCGGGC |
| 18 | ST8SIA1 PCR primer | GCTGCGGCCGCCTAGGAAGTGGGCTGGAGTG |
| | (G3DS) | |
| 19 | self-cleaving 2A | QCTNYALLKLAGDVESNPGP |
| | peptide of equine | |
| | rhinitis A virus | |
| 20 | GD3S/self-cleaving 2A | ATAGCGGCCGCATGAGCCCCTGCGG |
| | peptide/GD2S PCR | |
| | primer | |
| 21 | GD3S/self-cleaving 2A | GCTCTCTAGATCACTCGGCGGTCATGCAC |
| | peptide/GD2S PCR | |
| | primer | |
| 22 | exon 2-specific target | TTGCATTTATCGGAGATTCCAGG |
| | sequence | |
| 23 | Target region PCR | GCTGTGCCTAACAGTTTG |
| | primer | |
| 24 | Target region PCR | TGGCAAGTTTTTCCATGAG |
| | primer | |
| 25 | Target region PCR | TGAAGCAAAGAATTGCCTTGTAGA |
| | primer | |
| 26 | Target region PCR | CTTATTTCCTTCTTCTTTAAACTGGG |
| | primer | |
| 27 | CASD1 PCR primer | ATGTTCACAACGCCACGG |
| 28 | CASD1 PCR primer | CAGGAACCATCCACAGGC |
| 29 | NeuD PCR primer | CGCCGCGGATCCGAAAAAATAACCTTAAAATGC |
| 30 | NeuD PCR primer | GTCCGCTCGAGTTAAAATAGATTAAAAATTTTTTTTGATTTTAG |
| 31 | CASD1 PCR primer | GTGGATTTTCTGTGGCATCC |
| 32 | CASD1 PCR primer | AAGCGCTTCACTGCTACCAT |
| 33 | B4GALNT1 PCR | CAGCGCTCTAGTCACGATTGC |
| | primer (G2DS) | |
| 34 | B4GALNT1 PCR | CCACGGTAACCGTTGGGTAG |
| | primer (G2DS) | |
| 35 | ST8SIA1 PCR primer | GCGATGCAATCTCCCTCCT |
| | (G3DS) | |
| 36 | ST8SIA1 PCR primer | TTCCCGAATTATGCTGGGAT |
| | (G3DS) | |
| 37 | 8B6 mAb LC-CDR1 | QSLLKNNGNTFL |
| 38 | 8B6 mAb LC-CDR3 | SQSTHIPYT |
| 39 | 8B6 mAb HC-CDR1 | EFTFTDYY |
| 40 | 8B6 mAb HC-CDR2 | IRNRANGYTT |
| 41 | 8B6 mAb HC-CDR3 | ARVSNWAFDY |
| 42 | Humanized anti- | D/EV/IVMTQSPL/AS/TLP/SV/L/AS/TL/P/VGD/Q/EQ/P/RA/VS/TI/LS/TCRS/ |
| | OAcGD2 VL | ASQSL/VL/VKN/SN/QG/A/SN/Y/ST/N/SF/YLH/N/S/A/Y/GWY/FL/QQK/RPGQ/ |
| | consensus sequence | KS/A/VPK/Q/RL/R/VLIYK/G/LV/A/GSN/TRL/D/AS/TGV/IPD/A/ |
| | | SRFSGSGSGTY/DFTLK/TIS/NR/SV/LE/QA/PEDL/V/FG/AV/TYF/YCS/M/QQS/ |
| | | AT/YH/Q/NI/T/QP/SYTFGG/QGTKVEIK |
| 43 | Humanized anti- | E/QVQLV/LESGGGLVQ/KPGG/RSLRLSCA/TT/ASE/GFTFT/S/GDY/HYMT/ |
| | OAcGD2 VH | H/N/SWV/IRQAPGKGLEWL/VG/SF/YI/TRNR/K/SA/SN/SG/A/SY/GT/IT/ |
| | consensus sequence | IE/YYN/AP/A/DSVKGRFTISRDN/GS/AKS/NI/S/TL/T/AYLQMNSLR/K/QT/ |
| | | AEDTAV/I/LYYCA/TRVSNWA/YFDYWGQGTT/LL/VTVSS |
| 44 | Anti-OAcGD2 VH49A | EVQLVESGGGLVQPGRSLRLSCTTSEFTFTDYYMTWVRQAPGKGLEWL |
| | | GFIRNRANGYTTEYNPSVKGRFTISRDNSKSILYLQMNSLKTEDTAVYYC |
| | | ARVSNWAFDYWGQGTLVTVSS |
| 45 | Anti-OAcGD2 VH72A | EVQLVESGGGLVQPGGSLRLSCATSEFTFTDYYMTWVRQAPGKGLEWL |
| | | GFIRNRANGYTTEYNPSVKGRFTISRDNSKNSLYLQMNSLKTEDTAVYY |
| | | CARVSNWAFDYWGQGTLVTVSS |
| 46 | Anti-OAcGD2 | EVQLVESGGGLVQPGRSLRLSCTTSEFTFTDYYMTWVRQAPGKGLEWL |
| | VH49BHS | GFIRNKANGYTTEYNPSVKGRFTISRDNSKSILYLQMNSLKTEDTAVYYC |
| | | ARVSNWAFDYWGQGTLVTVSS |
| 47 | Anti-OAcGD2 | EVQLVESGGGLVQPGGSLRLSCATSEFTFSDYYMTWVRQAPGKGLEWL |
| | VH72BHNPS | GFIRNKANGYTTEYNPSVKGRFTISRDNSKNSLYLQMNSLKTEDTAVYY |
| | | CARVSNWAFDYWGQGTLVTVSS |
| 48 | Anti-OAcGD2 VL30A | DVVMTQSPLSLPVTLGQPASISCRSSQSLLKNNGNTFLHWYQQRPGQSPR |
| | | LLIYKVSNRLSGVPDRFSGSGSGTDFTLKISRVEAEDVGVYFCSQSTHIPY |
| | | TFGGGTKVEIK |
| 49 | Anti-OAcGD2 VL28A | DVVMTQSPLSLPVTPGEPASISCRSSQSLLKNNGNTFLHWYLQKPGQSPQ |
| | | LLIYKVSNRLSGVPDRFSGSGSGTDFTLKISRVEAEDVGVYFCSQSTHIPY |
| | | TFGQGTKVEIK |
| 50 | Anti-OAcGD2 | DVVMTQSPLSLPVTPGEPASISCRSSQSLLKSNANTFLHWYLQKPGQSPQ |
| | VL28Bs01/A2 | LLIYKVSNRLSGVPDRFSGSGSGTDFTLKISRVEAEDVGVYYCMQSTHIP |
| | | YTFGQGTKVEIK |
| 51 | Humanized anti- | DV/IVMTQSPLSLPVS/TL/PGD/Q/EQ/PASISCRSSQSLL/VKN/SNG/ANTF/ |
| | OAcGD2 VL | YLHWY/FL/QQK/RPGQSPK/Q/RLLIYKVSNRL/ASGVPDRFSGSGSGTY/ |
| | consensus sequence | DFTLKISRVEAEDL/VGVYF/YCS/MQSTHIPYTFGG/QGTKVEIK |
| 52 | Humanized anti- | EVQLVESGGGLVQPGG/RSLRLSCA/TT/ASE/GFTFT/S/GDY/HYMT/ |
| | OAcGD2 VH | SWVRQAPGKGLEWLGFIRNR/KAN/SG/S/AY/GTT/IE/YYN/AP/ |
| | consensus sequence | ASVKGRFTISRDNSKS/NI/SL/AYLQMNSLR/KTEDTAVYYCA/ |
| | | TRVSNWAFDYWGQGTT/LL/VTVSS |
| 53 | EZH2 human protein | MGQTGKKSEKGPVCWRKRVKSEYMRLRQLKRFRRADEVKSMFSSNRQ |
| | | KILERTEILNQEWKQRRIQPVHILTSVSSLRGTRECSVTSDLDFPTQVIPLK |
| | | TLNAVASVPIMYSWSPLQQNFMVEDETVLHNIPYMGDEVLDQDGTFIEE |
| | | LIKNYDGKVHGDRECGFINDEIFVELVNALGQYNDDDDDDDGDDPEERE |
| | | EKQKDLEDHRDDKESRPPRKFPSDKIFEAISSMFPDKGTAEELKEKYKEL |
| | | TEQQLPGALPPECTPNIDGPNAKSVQREQSLHSFHTLFCRRCFKYDCFLH |
| | | RKCNYSFHATPNTYKRKNTETALDNKPCGPQCYQHLEGAKEFAAALTA |
| | | ERIKTPPKRPGGRRRGRLPNNSSRPSTPTINVLESKDTDSDREAGTETGGE |
| | | NNDKEEEEKKDETSSSSEANSRCQTPIKMKPNIEPPENVEWSGAEASMFR |
| | | VLIGTYYDNFCAIARLIGTKTCRQVYEFRVKESSIIAPAPAEDVDTPPRKK |
| | | KRKHRLWAAHCRKIQLKKDGSSNHVYNYQPCDHPRQPCDSSCPCVIAQ |
| | | NFCEKFCQCSSECQNRFPGCRCKAQCNTKQCPCYLAVRECDPDLCLTCG |
| | | AADHWDSKNVSCKNCSIQRGSKKHLLLAPSDVAGWGIFIKDPVQKNEFIS |
| | | EYCGEIISQDEADRRGKVYDKYMCSFLFNLNNDFVVDATRKGNKIRFAN |
| | | HSVNPNCYAKVMMVNGDHRIGIFAKRAIQTGEELFFDYRYSQADALKY |
| | | VGIEREMEIP |
| 54 | HPRT PCR primer | GCCAGACTTTGTTGGATTTG |
| | (forward) | |
| 55 | HPRT PCR primer | CTCTCATCTTAGGCTTTGTATTTTG |
| | (reverse) | |

In the consensus sequences of SEQ ID NO: 42, 43, 51 and 52, “residuel/residue2” at a given position means that the residue at that position is either residuel or residue2. CDRs are represented in bold.

### EXAMPLES

The present invention is further illustrated by the following examples.

### Example 1: CASD1 is Essential for O-Acetylation of GD2

### Materials and Methods

### Antibodies

The anti-GD3 R24 mouse IgG3 was purchased from Abcam (Cambridge, MA, USA). The mouse IgM anti-9-OAcGD3 mAb M-T6004 was from Thermo Scientific (Waltham, USA). The anti-GD2 mAb 14.18 mouse IgG3/k and the anti-OAcGD2 mAb 8B6 mouse IgG3/k were produced in CHO cells by OGD2 Pharma (Nantes, France). The mouse IgG2a anti-GD2 mAb ME361 used for immune-TLC experiments was from Kerafast (Winston-Salem, USA). The secondary antibodies Alexa Fluor 488 donkey anti-mouse IgG and Alexa Fluor 546 donkey anti-rabbit IgG were purchased from Invitrogen (Cergy Pontoise, France).

### Mammalian Expression Plasmids

To generate a construct encoding full-length CASD1 with an N-terminal V5 and a C-terminal Myc epitope (V5-CASD1-Myc), the coding region of human CASD1 (accession no. NM_022900) was amplified by PCR using the primers 5′-GCTCGGGATCCGCGGCTCTGGCCTACAACCTG-3′ (SEQ ID NO: 9) and 5′-GCTCGCTCGAGATGTTTTGATTTATCTTGAATGGATG-3′ (SEQ ID NO: 10) containing BamHI and XhoI restriction sites (underlined), respectively, and the resulting PCR product was ligated into the corresponding restriction sites of the vector pcDNA3 (Invitrogen). Sequences encoding the epitope tags were inserted by adapter ligation. For the V5 epitope, the pre-hybridized oligonucleotide pair 5′-AGCTTCGAATGGGTAAGCCTATCCCTAACCCTCTCCTCGGTCTCGATTCTACGG-3′ (SEQ ID NO: 11) and 5′-GATCCCGTAGAATCGAGACCGAGGAGAGGGTTAGGGATAGGCTTACCCATTCG A-3′ (SEQ ID NO: 12) was ligated into the HindIII and BamHI sites of pcDNA3. For the Myc epitope, the pre-hybridized oligonucleotide pair 5′-TCGAGGAACAAAAACTCATCTCAGAAGAGGATCTGAATTAAT-3′ (SEQ ID NO: 13) and 5′-CTAGATTAATTCAGATCCTCTTCTGAGATGAGTTTTTGTTCC-3′ (SEQ ID NO: 14) was ligated into the XhoI and XbaI sites of pcDNA3, resulting in the plasmid pcDNA3-V5-CASD1(wt)-Myc.

Site-directed mutagenesis was performed by PCR using the QuikChange site-directed mutagenesis kit (Stratagene) and pcDNA3-V5-CASD1(wt)-Myc as template. To introduce the amino-acid exchange S94A, the mutagenesis primers 5′-GCATTTATTGGAGATGCCAGAATTCGTCAATTG-3′ (SEQ ID NO: 15) and 5′-CAATTGACGAATTCTGGCATCTCCAATAAATGC-3′ (SEQ ID NO: 16) were used, resulting in the plasmid pcDNA-V5-CASD1(S94A)-Myc.

For expression of the human sialyltransferase ST8SIA I, a full-length construct encoding an N-terminal V5 epitope was generated by amplification of the coding region of human ST8SIA1 (I.M.A.G.E. clone IRCMp5012B0613D, ImaGenes) with the primers 5′-GCTAAGCTTCGAATGGGTAAGCCTATCCCTAACCCTCTCCTCGGTCTCGATTCT ACGGGTACCAGCCCCTGCGGGCGGGC-3′ (SEQ ID NO: 17) and 5′-GCTGCGGCCGCCTAGGAAGTGGGCTGGAGTG-3′ (SEQ ID NO: 18) containing the sequence encoding the V5 epitope (bold), HindIII and NotI restriction sites (underlined). The resulting PCR fragment was ligated into the HindIII and NotI sites of the expression vector pcDNA3.1-zeo (Invitrogen). For efficient co-expression of GD3S and GD2S, the inventors generated a plasmid that carries the coding sequence of GD3S (accession no. NM_011374.2) without stop-codon fused to a sequence stretch that encodes the self-cleaving 2A peptide of equine rhinitis A virus (QCTNYALLKLAGDVESNPGP (SEQ ID NO: 19)) and the coding sequence of GD2S (accession no. NM_008080.5). The entire tripartite sequence was generated by gene synthesis (Eurofins MWG Operon), amplified by PCR using the primers 5′ -ATAGCGGCCGCATGAGCCCCTGCGG-3′ (SEQ ID NO: 20) and 5′ -GCTCTCTAGATCACTCGGCGGTCATGCAC-3′ (SEQ ID NO: 21), and the obtained PCR product was ligated into the Notl and Xbal restriction sites of the vector pcDNA3 (Invitrogen). The identity of the final construct was verified by sequencing.

### Mammalian Cell Culture

Cell culture reagents were purchased from Lonza (Verviers, Belgium). The human breast cancer cell SUM159PT was obtained by the American Tissue Culture Collection (ATCC, Rockville, MD, USA). Cells were routinely grown in monolayer culture and maintained at 37° C. in an atmosphere of 5% CO2. Chinese Hamster Ovary (CHO) cells were cultivated in Dulbecco's Modified Eagle's Medium (DMEM)/Ham's F12 1:1 (PAN-Biotech) supplemented with 5% fetal calf serum (FCS) (Sigma-Aldrich) and maintained at 37° C. and 5% CO2. SUM159PT cells were grown in DMEM/F12 (1:1) containing 5% heat-inactivated fetal calf serum (FCS), 2 mM L-glutamine, 1 μg/mL hydrocortisone and 5 μg/mL insulin.

### Transfection of CHO Cells

For transient transfections, CHO cells were cultivated in 10 cm dishes until they reached 70-80% confluency. A mixture of 12 μl PEI MAX (Polysciences) and 12 μg of plasmid DNA was prepared in 1.2 ml Opti-MEM (Gibco), incubated for 20 mM at room temperature and added drop-wise to a cell culture containing 12 ml of culture medium. After 6 h, transfections were stopped by removal of the transfection mixture and the addition of fresh culture medium. Transfections in 24-well plates were performed accordingly using a mixture of 0.5 μl PEI and 0.5 μg DNA in 50 μl of OptiMEM that was added to cells maintained in 500 μl of culture medium.

siRNA Transfection of SUM159PT Cells

Depletion of CASD1 was performed using siRNA strategy by a double transfection. The second transfection was performed 48 h after the first one using the same conditions. Cells were grown in six-well plates and transfections were performed with 2 μM of siRNA-targeting CASD1 (L-016926-01-0010, Horizon) or a scramble sequence and 8 μL RNAimax (#137781, Thermo-Fisher Scientific) in 1 mL of UltraMem (Lonza). After 5 h, transfection was stopped by adding 1 mL of DMEM/F12 media supplemented with 5% FCS. Cells were collected at 72 h for quantitative polymerase chain reaction (qPCR) and immunocytochemistry experiments.

shRNA Transfection of SUM159PT Cells

Stable depletion of CASD1 was performed using shRNA strategy. ShRNA encoding plasmids were from EZyvec (Loos, France). Cells were grown in six-well plates and transfection was performed with 500 ng of shRNA plasmid targeting-CASD1 (A236.1b) or a scramble sequence in 4 μL lipofectamine 2000 (Invitrogen). The selection of stable transfectants was performed by adding hygromycin at 500 μg/ml 48 h after transfection.

Transfection of SUM159PT Cells with CASD1 or GD3 Synthase-Encoding Expression Vector

Transfection of SUM159PT cells was performed with RNAimax transfection reagent (#137781, Thermo-Fisher Scientific). Cells were grown in six-well plates, washed twice with UltraMem and transfected with 2 μg of plasmid DNA and 4 μL of RNAimax in 1 mL of UltraMem (Lonza). After 5 h, transfection was stopped by adding 1 mL of DMEM/F12 media supplemented with 5% of FCS. For the selection of stable transfectants, 500 μg/mL of hygromycin was added per well 48 h post-transfection. Clones were isolated by limited dilution. Positive clones were selected by qPCR and immunocytochemistry-confocal microscopy experiments.

### CRISPR/Cas-Mediated Genome Editing

CHO cells carrying a selective Casd1 gene knockout (CHOΔCasd1) were generated by introducing a frameshift mutation in exon 2 of Casd1 by CRISPR/Cas9-mediated genome editing. Exon 2 of hamster Casd1 corresponds to exon 3 of human CASD1 and encodes the active site serine. A plasmid encoding a respective Casd1-specific guide RNA was generated on the basis of the bicistronic vector pX330-U6-Chimeric_BB-CBh-hSpCas9 (Addgene plasmid # 42230; http://n2t.net/addgene:42230; RRID:Addgene_42230). Following the protocol provided in Cong et al., 2013, Science 339, 819-23, the exon 2-specific target sequence 5′-TTGCATTTATCGGAGATTCCAGG-3′ (PAM sequence underlined) (SEQ ID NO: 22) was inserted into the Bbsl sites of the vector. The final plasmid allowed co-expression of the RNA-guided nuclease Cas9 from Streptococcus pyogenes and the Casd1-specific guide RNA. Transient transfections in CHO cells were performed in 24-well plates using 0.375 jag of the CRISPR/Cas9-plasmid and 0.125 μg of a reporter plasmid (pEGFP-C1, Clontech) that allowed expression of the enhanced green fluorescent protein (EGFP). After 24 h, cells were cloned by limiting dilution and colonies grown from EGFP-expressing single-cell clones were expanded and screened for frameshift mutations. This included amplification of the target region by PCR using two primer sets (5′-GCTGTGCCTAACAGTTTG-3′ (SEQ ID NO: 23)/5′-TGGCAAGTTTTTCCATGAG-3′ (SEQ ID NO: 24) and 5′-TGAAGCAAAGAATTGCCTTGTAGA-3′ (SEQ ID NO: 25)/5′-CTTATTTCCTTCTTCTTTAAACTGGG-3′ (SEQ ID NO: 26)) and sequencing of the obtained PCR product. CHO clones carrying homozygous or heterozygous frameshift mutations in exon 2 of Casd1 were subcloned by limiting dilution and re-analyzed. In this step, frameshift mutations were confirmed on the genomic level as described above and additionally verified on the transcript level by amplification of Casd1 transcripts by RT-PCR and analysis of the PCR products by sequencing. As gene-specific primers, the following multiple intron-spanning primer pair was used: 5′-ATGTTCACAACGCCACGG-3′ (exon 1) (SEQ ID NO: 27) and 5′-CAGGAACCATCCACAGGC-3′ (exon 8) (SEQ ID NO: 28). The CHOΔCasd1 clone used in this study contains a 2 bp insertion on one allele and a 4 bp deletion on the second allele. Both frameshift mutations occurred at the 5′-end of the triplet encoding Asp-60. This eliminated the triplet that encodes the catalytic residue Ser-61 and resulted in the formation of a premature stop codon in exon 2.

Production of the Sialyl-9-O-Acetyltransferase NeuD of Campylobacter jejuni

The coding sequence of NeuD (orf11) was amplified from genomic DNA of the Campylobacter jejuni (C. jejuni) strain MK104 (ATCC 43446) in a PCR reaction with the primers 5′-CGCCGCGGATCCGAAAAAATAACCTTAAAATGC-3′ (SEQ ID NO: 29) and 5′-GTCCGCTCGAGTTAAAATAGATTAAAAATTTTTTTTGATTTTAG-3′ (SEQ ID NO: 30). The obtained PCR product was ligated into the BamHI and XhoI sites of a pET32a (Novagen) vector that carries a sequence encoding the maltose binding protein (MBP), an (S)3(N)10-linker and a thrombin cleavage site (LVPRGS) that was inserted into the NdeI and Xhol sites, with the last two triplets encoding the most C-terminal amino acids of the cleavage site (GS) creating a unique BamHI restriction site. The identity of the resulting construct was confirmed by sequencing and the encoded MBP-NeuD fusion protein was expressed in E. coli BL21(DE3). Transformed cells were cultivated at 37° C. in Power Broth (AthenaES) until an optical density at 600 nm of 1.5 was reached. The expression was induced with 1mM isopropyl-β-D-thiogalactopyranoside (IPTG) and cultivation at 15° C. for 20 h. Cells were harvested and resuspended in binding buffer (20 mM Tris-HCl pH 7.4, 200 mM NaCl, 1 mM EDTA) containing 40 μg/ml bestatin, 1 μg/ml pepstatin and 1 mM PMSF, and were disrupted by sonication. Recombinant protein was purified on 1 ml MBPTrap HP columns (GE Healthcare) using 10 mM D-(+)-maltose in binding buffer for elution. Affinity purified protein was dialyzed against 50 mM MES pH 7.0 containing 100 mM NaCl (Slide-A-Lyzer, ThermoFisher, 3.5 kDa cutoff) and concentrated using an Amicon Ultra-4 centrifugal filter device (Merck Millipore, 50 kDa cutoff).

### Generation of 9-O-Acetylated Gangliosides as Standards for TLC

The 9-O-acetylated forms of GD2 and GD3 were generated by enzymatic in vitro synthesis using NeuD from C. jejuni, which allows the site-selective introduction of an O-acetyl group at position C9 of a terminal α2,8-linked sialic acid. GD3 (Sigma-Aldrich, 345752) and GD2 (Sigma-Aldrich, 345743) gangliosides (1 mM) were dissolved in MES buffer (50 mM) pH 6.5 or pH 7, containing acetyl-Coenzyme A (1 mM), MgCl₂ (10 mM) and dithiothreitol (1 mM) (Fluka, Buchs, Germany) Various concentrations of sodium cholate (Sigma-Aldrich, Steinheim, Germany), ranging from 0 to 0.2% (w/v) were added to the reaction. NeuD (100 mU) was added and the reaction mixture was incubated at 37° C. for 3 hours with stirring (300 rpm). The reaction was stopped by adding an equal volume of methanol and gangliosides were purified on Chromabond C18 columns (Macherey-Nagel), dried under a nitrogen stream and dissolved in chloroform/methanol (1:2, v/v).

### Extraction of Gangliosides

Total gangliosides were extracted from transfected CHO cells by mixing 107 cells with 3 ml chloroform/methanol (1:2, v/v) and sonic dispersion. After twenty pulses given by a Sonifier S-450 equipped with a cup horn (Branson), samples were incubated for 15 min in a bath sonicator. Debris were removed by centrifugation (1,600×g for 10 min) and the supernatant was transferred into a new tube. After adjusting a final ratio of chloroform/methanol/water of 4:8:5 (v/v/v), samples were centrifuged (1,600×g for 10 min) and the upper phase containing the ganglioside fraction was desalted on a Chromabond C18 column (Macherey-Nagel). Gangliosides were dried under a nitrogen stream, dissolved in 20 μl of chloroform/methanol (1:2, v/v) and stored at −20° C.

### High-Performance Thin-Layer Chromatography (HPTLC) and Immunostaining

Total gangliosides of an equivalent of 2×10⁶ cells or 0.2 μg of the indicated ganglioside standards were spotted on Nano-DURASIL-20 (0.2 mm silica gel 60) HPTLC plates (Macherey-Nagel) and chromatographed in chloroform/methanol/H₂O (50:40:10, v/v/v) containing 0.05% calcium chloride. HPTLC plates were dried and chromatographed twice in 0.5% poly(isobutyl methacrylate) (Sigma-Aldrich) in hexane, which was prepared from a 25% stock solution in chloroform (w/v). Plates were dried and incubated overnight at 37° C. in PBS. After blocking with 2% BSA (w/v) in PBS for 1 h at room temperature, plates were incubated with the following primary antibodies diluted in PBS: Mouse IgG3 anti-9-OAcGD2 mAb 8B6 (10 μg/ml; OGD2 Pharma), mouse IgM anti-9-OAcGD3 mAb M-T6004 (1:40; Thermo Scientific, MA1-34707), mouse IgG2a anti-GD2 mAb ME361 (15 μg/ml; Kerafast EWI023), or mouse IgG3 anti-GD3 mAb R24 (10 μg/ml; purified by protein A affinity chromatography from cell culture supernatant of R24 hybridoma cells ATCC HB-8445). HPTLC plates were washed three times with PBS and incubated for 1 h at room temperature with goat anti-mouse IgM IRDye 800CW-conjugate (1:20,000; LI-COR Biosciences, 926-32280) or goat anti-mouse IgG IRDye 800CW-conjugate (1:10,000; LI-COR Biosciences, 926-32210). HPTLC plates were washed with PBS and bound antibodies were detected by infrared imaging using an Odyssey Imaging System (LI-COR Biosciences).

RNA Extraction, cDNA Synthesis and qPCR

Gene expression was evaluated using real-time qPCR analysis after RNA extraction and cDNA synthesis. Total RNA was extracted using the Nucleospin RNA II kit (Macherey-Nagel, Duren, Germany) The amount of extracted RNA was quantified using a DeNovix DS-11 spectrophotometer (DeNovix Inc., Wilmington, DE, USA) and the purity of the RNA was checked by the ratio of the absorbance at 260 and at 280 nm. Total RNA was subjected to reverse transcription using the Maxima First Strand cDNA Synthesis Kit (ThermoFisher Scientific, Villeneuve d'Ascq, France) according to the protocol provided by the manufacturer. The oligonucleotide sequences (Eurogentec, Seraing, Belgium) used as primers for the PCR reactions are given in SEQ ID NO: 31 and SEQ ID NO: 32. qPCR and subsequent data analysis were performed using the Mx3005p Quantitative System (Stratagene, La Jolla, CA, USA). PCR reaction (25 μL) contained 12.5 μL of the 2X Brilliant SYBR Green qPCR Mastermix (Thermo Fischer Scientific, Rockford, USA), 300 nM of primers and 4 μL of cDNA (1:40). DNA amplification was performed with the following thermal cycling profile: initial denaturation at 94° C. for 10 min, 40 cycles of amplification (denaturation at 94° C. for 30 s, annealing at Tm for 30 s, and extension at 72° C. for 30 s) and a final extension at 72° C. for 5 min. Hypoxanthineguanine PhosphoRibosylTransferase (HPRT) gene was used to normalize the expression of genes of interest. The fluorescence monitoring occurred at the end of each cycle. The analysis of amplification was performed using the Mx3005p software. The specificity of the amplification was checked by recording the dissociation curves. The efficiency of amplification was checked by serial dilutions of cDNA from SK-MEL-28 cells and was between 97 and 102%. All experiments were performed in triplicate. The quantification was performed by the method described by Pfaffl (Pfaffl, M.W., 2001, Nucleic Acids Res. 29(9), e45).

### Immunocytochemistry and Confocal Microscopy

Transfected cells were grown on glass coverslips fixed for 15 min in 4% paraformaldehyde in 0.1 M sodium phosphate buffer. Cells were washed thrice with PBS and membrane permeabilization was performed in 5 μg/mL digitonin in PBS for 20 min. After saturation in blocking buffer, cells were incubated with either with the anti-GD2 or anti-OAcGD2, or anti-V5-tag mAbs at 20 μg/mL for 1 h followed by the secondary antibody for 1 h. Cells were washed and mounted in fluorescent mounting medium (Dako, Carpetaria, CA, USA). Stained slides were analyzed under a Zeiss LSM 700 confocal microscope. The same settings were used for all acquisitions to ensure the comparability of the data obtained.

### MTS Assay

Cell growth was analyzed using the MTS reagent (Promega, Charbonnières-les-bains, France) according to the manufacturer's instructions. Briefly, cells were seeded in 96 well plates in 0%, 1% or 5% FCS containing media in which MTS reagents were added. The proliferation rate was measured by the absorbance of MTS reagent at 490 nm at 24 h, 48 h, 72 h, and 96 h after seeding.

### Transwell Assays

Migration and invasion properties of cells were measured by transwell assays using migration chambers or invasion chambers (Dutscher, Brumath, France). Cells were seeded in 24-well plates containing either migration or invasion chambers in serum-free media. After 24 h incubation at 37° C., cells were fixed 4% paraformaldehyde in 0.1 M sodium phosphate buffer and non-migratory/invasive cells were swapped with cotton swabs. Nuclei were counterstained with DAPI and membrane were mounted on the slide with fluorescent mounting medium (Dako, Carpetaria, CA, USA). Nuclei were counted under Leica microscope.

### Statistical Analysis

Statistical difference was assessed using unpaired t-test or ordinary one-way Anova.

### Results

### CASD1 is Essential for the 9-O-Acetylation of GD2

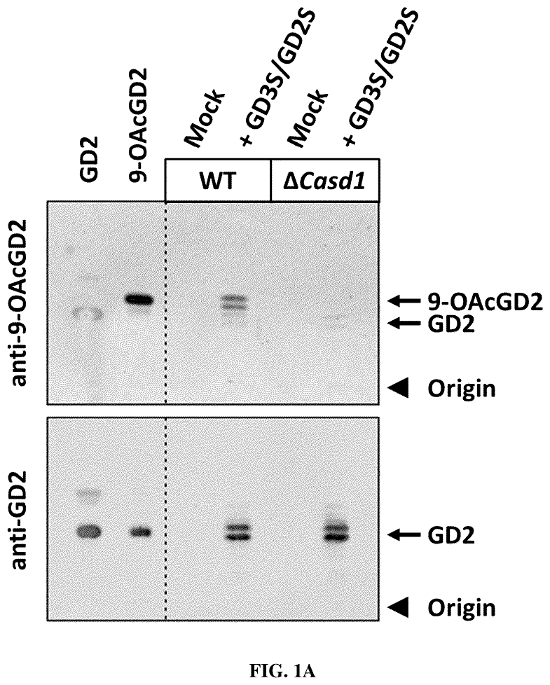

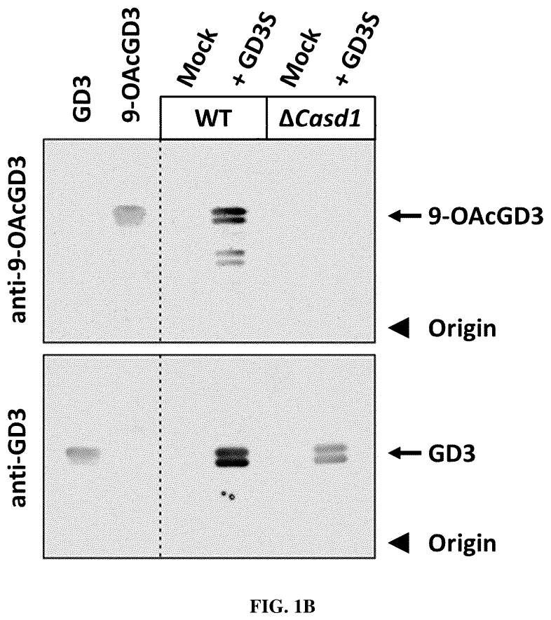

Prior to deciphering the role of CASD1 in breast cancer cells, the inventors dissected the biosynthesis of 9-OAcGD2 in CHO cells, a well-defined cellular system. CHO cells display mainly the mono-sialyl ganglioside GM3, are easy to transfect and known to produce 9-OAcGD3 upon expression of GD3S. Using CRISPR/Cas9-mediated genome editing, the inventors generated CHOΔCasd1 cells by introducing a frameshift mutation in exon 2. To produce GD2, CHO wild type (WT) and CHOΔCasd1 cells were transiently transfected with a bicistronic plasmid that allows co-expression of GD3S and GD2S. Total gangliosides were extracted from transfected cells and analyzed by thin-layer chromatography (TLC). Upon transfection of the expression plasmid, but not of empty vector (mock), GD2 was detected in both CHO-WT and CHOΔCasd1 cells (FIG. 1A, lower panel). The formation of 9-OAcGD2 was observed in CHO-WT, but not in Casd1-deficient cells (FIG. 1A, upper panel), demonstrating that the biosynthesis of 9-OAcGD2 critically relies on CASD1. In addition, the formation of GD3 and 9-OAcGD3 in GD3S expressing CHO cells was monitored (FIG. 1B). The deletion of Casd1 in CHO cells also prevented the formation of 9-OAcGD3.

### CASD1 Expression is Ubiquitous Among Breast Cancer Cells

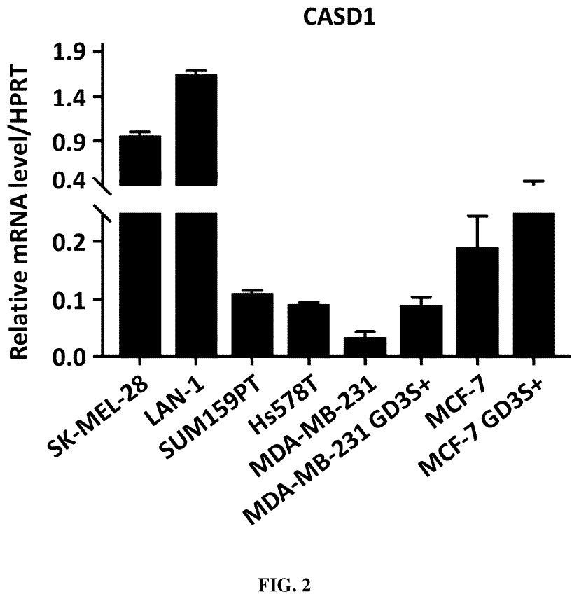

Results in CHO cells suggest that the expression of 9-OAcGD2, the major O-acetylated ganglioside species in breast cancer cells, is CASD1 dependent. CASD1 expression in breast cancer cells was next studied. The human protein atlas reveals that CASD1 is expressed in almost all healthy and cancer tissues (http://www.proteinatlas.org/ENSG00000127995 -CASD1/tissue). qPCR experiments were performed in order to quantify the expression of CASD1 in different breast cancer cells. The inventors used SUM159PT, Hs578T, and 2 clones derived from MDA-MB-231 (MDA-MB-231 GD3S+) and MCF-7 (MCF-7 GD3S+) breast cancer cell lines overexpressing GD3 synthase and high levels of complex gangliosides. SK-MEL-28 melanoma cells and LAN-1 neuroblastoma cells expressing high levels of O-acetylated gangliosides were used as controls. The results presented in FIG. 2 indicate that CASD1 expression is ubiquitous among breast cancer cells confirming the human protein atlas data. Among breast cancer cell lines tested, CASD1 is more expressed in MCF-7 and MCF-7 GD3S+, compared to MDA-MB-231, MDA-MB-231 GD3S+SUM159PT and Hs578T cells. The level of CASD1 expression is distinctly higher in SK-MEL-28 and LAN-1 compared to breast cancer cells. SUM129PT, a triple negative breast cancer cell line derived from anaplastic carcinoma, was chosen for this study. The inventors' previous data show a moderate expression of GD2 and OAcGD2, and CASD1 (FIG. 2) suggesting that SUM149PT is suitable for both depletion and overexpression of CASD1.

### Reduction of CASD1 Expression

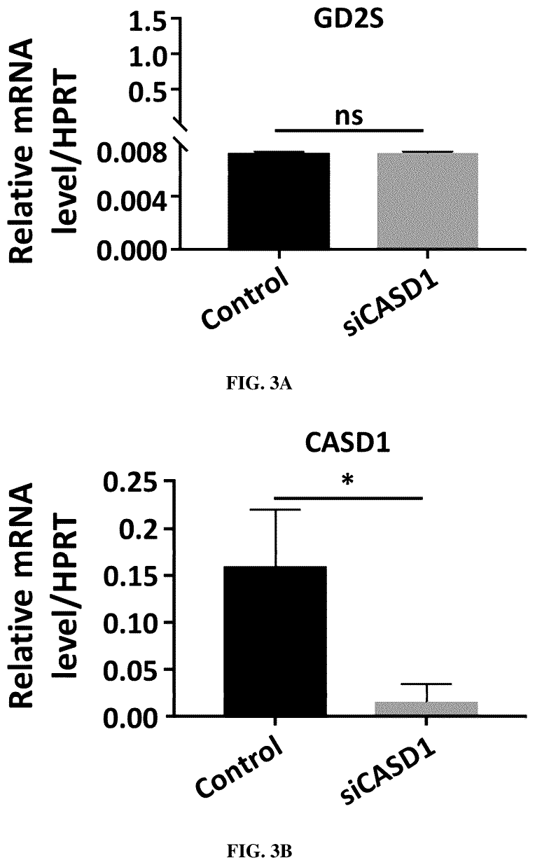

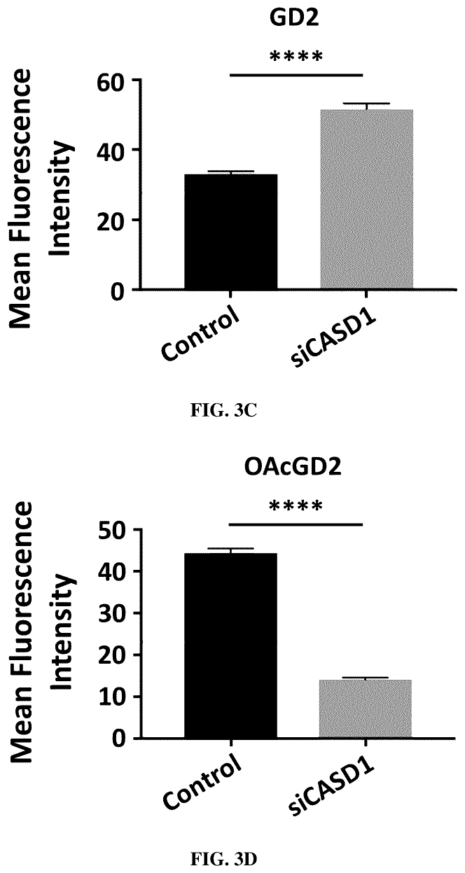

The reduction of CASD1 expression in SUM159PT was performed by transient transfection using siRNA strategy. The expression levels of GD2S (B4GALNT1) and CASD1 genes were determined by qPCR experiments and normalized to HPRT gene expression. Transfected cells exhibit a decrease of CASD1 gene expression (FIG. 3A) (up to 50%) while GD2 synthase gene expression is unchanged compared to control cells (FIG. 3B). The effect of CASD1 depletion on OAcGD2 expression was evaluated by immunofluorescence and confocal microscopy experiments. OAcGD2 expression was reduced in CASD1-depleted cells compared to control cells. The mean fluorescence intensity calculated based on multiple images showed that transfected cells exhibit an increased GD2 expression (FIG. 3C), but a 75% decrease in OAcGD2 expression compared to control cells (FIG. 3D). The inventors concluded that a 50% reduction of CASD1 gene expression lead to a 75% decrease of OAcGD2 expression in transiently transfected cells compared to SUM159PT control cells. The stable depletion of CASD1 expression using shRNA strategy was performed twice. Nevertheless, transfected cells did not grow after several passages in antibiotic-containing medium (data not shown) and stable CASD1 depletion could not be achieved in SUM159PT breast cancer cells.

### Transient Overexpression of CASD1 in SUM159PT Breast Cancer Cells

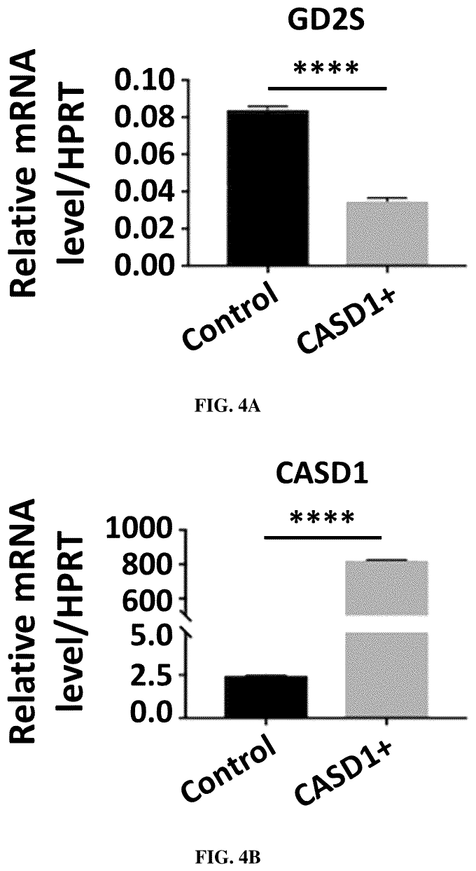

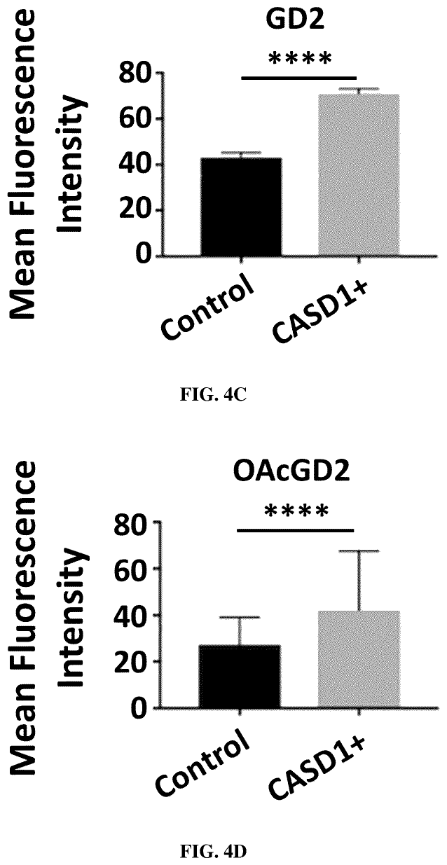

Overexpression of CASD1 (CASD1+) in SUM159PT cells was performed using a plasmid that allows the expression of human CASD1 with an N-terminal V5-epitope. In these experiments, CASD1 and GD2 synthase (GD2S) gene expression was assessed by qPCR experiments and the effect of CASD1 overexpression on OAcGD2 expression was studied by immunocytochemistry and confocal microscopy. CASD1 mRNA expression level showed approximately a 3000-fold increase in transfected cells compared to control cells (FIG. 4B). GD2 synthase expression remained unchanged between controls and transfected cells (FIG. 4A). The efficiency of transfection was checked using an anti-V5-tag antibody and ganglioside expression with either anti-GD2 or anti-OAcGD2 antibodies. CASD1 transfected cells exhibited an increase in OAcGD2 and GD2 expression compared to control cells. Mean fluorescence intensity quantified for each condition showed that overexpression of CASD1 increased both GD2 (FIG. 4C) and OAcGD2 (FIG. 4D) expression by 60% and 55%, respectively. The inventors concluded that, as observed for the transient inhibition of CASD1 gene expression, the transient overexpression of CASD1 in SUM159PT showed an effect on OAcGD2 expression. Since the stable depletion of CASD1 by shRNA in SUM159PT cells remained unsuccessful (data not shown), stable overexpression was considered.

### Stable Overexpression of CASD1 in SUM159PT Breast Cancer Cells

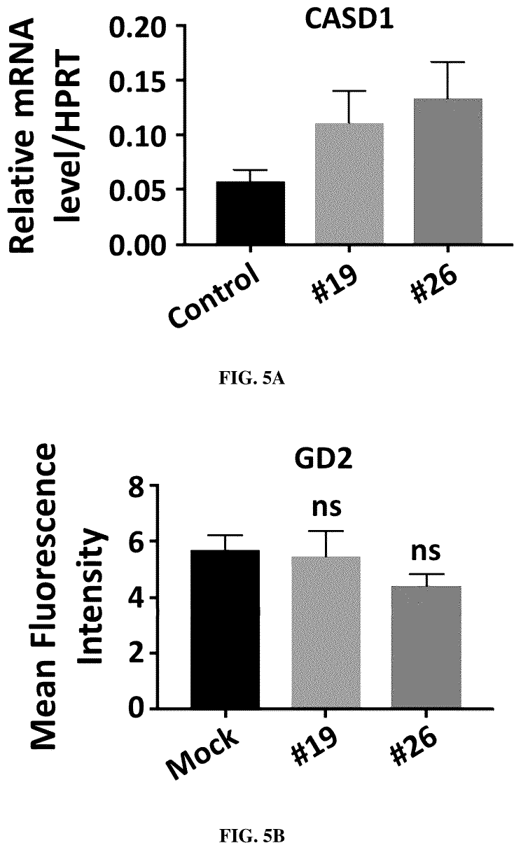

Stable transfectants overexpressing CASD1 (SUM159PT CASD1+) was produced using the plasmid pcDNA3.1 V5-tag-CASD1-cMyc and clones were isolated after antibiotic selection and limiting dilution cloning. From the 28 clones pre-selected, 12 clones were maintained during proliferation monitoring. CASD1 expression levels in these clones were assessed by qPCR experiments, confirming the overexpression of CASD1 in CASD1+clones compare to controls (data not shown). Selection of CASD1+clones among the 12 clones isolated has been performed by the analysis of GD2 and OAcGD2 expression using immunocytochemistry and confocal microscopy. Two CASD1+clones exhibiting high CASD1 gene expression and OAcGD2 ganglioside expression were used to study the biological properties. The level of expression of CASD1, OAcGD2 and GD2 of the two selected clones (clone #19 and clone #26) is depicted in FIG. 5. CASD1 mRNA expression was 2-fold and 3-fold-increased in clone #19 and in clone #26 compared to control cells, respectively (FIG. 5A). Mean fluorescence intensity quantified shows an increased level of OAcGD2 expression in clones #19 and #26 compared to the control (FIG. 5C), whereas the expression of GD2 remained unchanged (FIG. 5B).

### Biological Properties of SUM159PT CASD1+

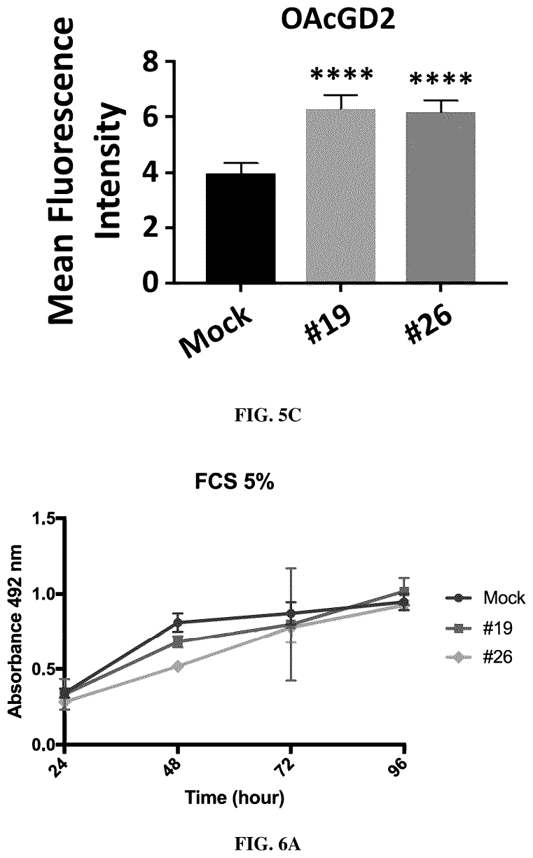

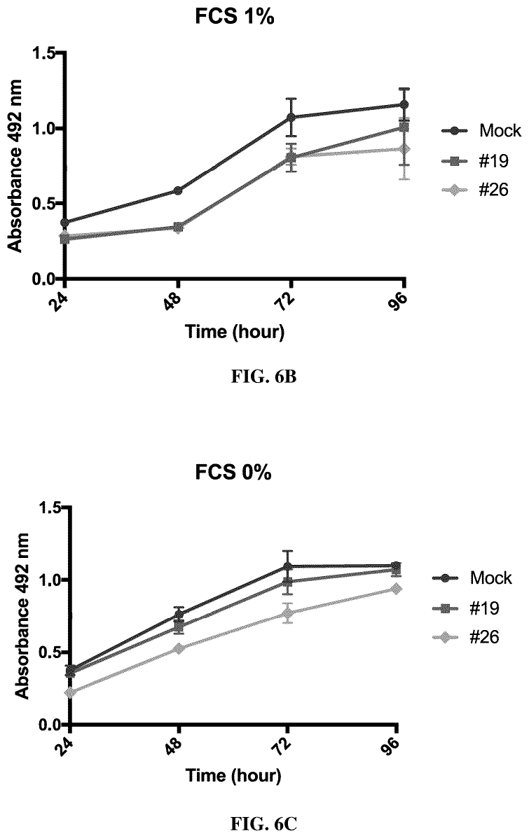

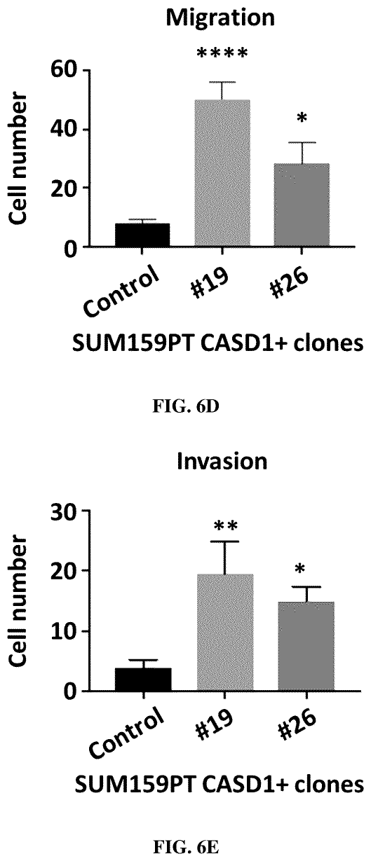

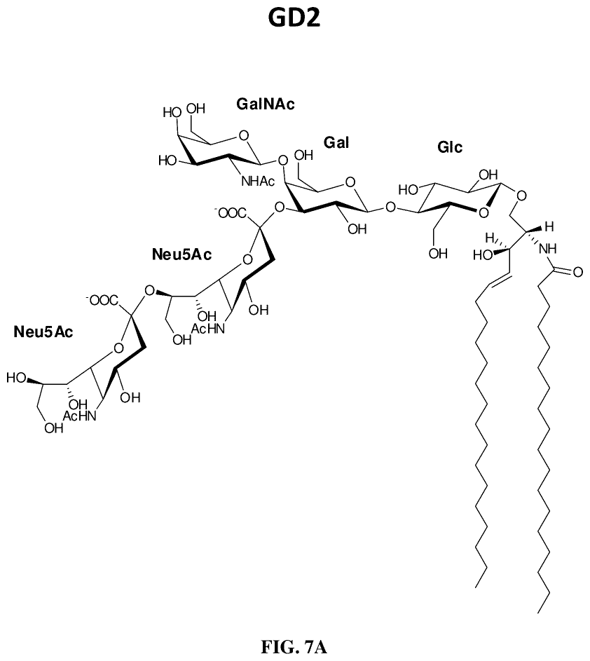

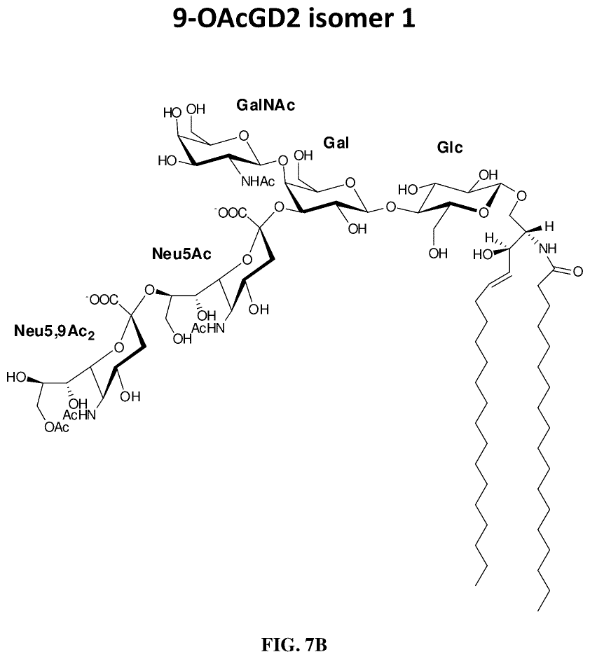

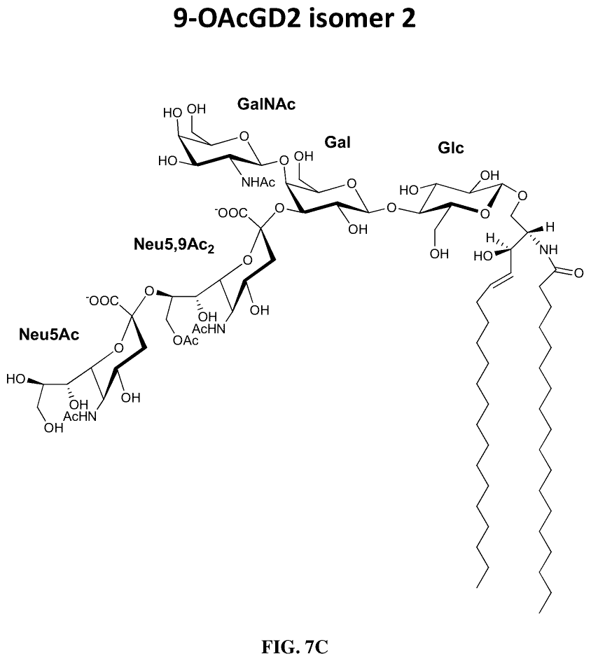

Biological properties of the SUM159PT CASD1+clones were studied by MTS and Transwell assays, to assess their proliferation and migration/invasion capabilities, respectively. SUM159PT CASD1+clones did not exhibit differential growth properties compared to their control counterpart, regardless of the percentage of fetal calf serum in the culture medium (FIGS. 6A, B, C). However, both clones showed increased migration (FIG. 6D) and invasion (FIG. 6E) capabilities in serum free media. The migration capabilities of SUM159PT CASD1+clones increased twice compared to their control counterpart (FIG. 6D). The invasion activity of clone #26 was doubled compared to control while this activity increased up to 10 folds in clone #19 compared to control (FIG. 6E).

### Discussion

Ganglioside O-acetylation results from the enzymatic action of a SOAT on a sialic acid residue. Recent studies have highlighted the importance of OAcGD2 as a marker and therapeutic target of interest in neuro-ectoderm derived cancers, including breast cancer. Deciphering GD2 O-acetylation mechanisms and the involvement of CASD1 in OAcGD2 biosynthesis in breast cancer is therefore of utmost importance.

### CASD1 is Involved in GD2 9-O-Acetylation in CHO Cells and in SUM159PT Cells.

In this study, the inventors first used CHO cell lines that do not naturally express b-series gangliosides, as a model to study CASD1 activity on gangliosides. Ganglioside expression can be modulated in these CHO cell lines, either by overexpressing GD3S required for GD3 expression, or both GD3S and GD2S for more complex ganglioside biosynthesis. Consequently, the CHO WT and CHOΔCasd1 cell lines are suitable models to study CASD1 SOAT activity on different gangliosides. The use of these cell lines allowed us to conclude that no O-acetylated ganglioside was detected in CHOΔCasd1 cells, highlighting the critical role of CASD1 in both GD3 and GD2 9-O-acetylation. These data also demonstrate that CASD1 is the unique SOAT involved in GD3 and GD2 9-O-acetylation in CHO cells.

Since there are no breast cancer cellular models available with a knockout for CASD1, the modulation of CASD1 expression was adopted as the strategy to assess the potential SOAT activity of CASD1 on GD2 O-acetylation in SUM159PT breast cancer cell line. Transient overexpression or depletion of CASD1 in SUM159PT cells modulated OAcGD2 expression: RNAi silencing of CASD1 induced a 70% decrease of OAcGD2 expression, whereas CASD1 overexpression increased OAcGD2 expression (50% increase). GD2 levels were either decreased (when CASD1 is overexpressed) or unchanged (when CASD1 is depleted). The inventors' previous structural analysis had allowed to identify 9-OAcGD2 as the major O-acetylated ganglioside species. Altogether, these data show that CASD1 is essential for GD2 9-O-acetylation in breast cancer cells, as demonstrated in CHO cells.

### Influence of CASD1 and OAcGD2 on Breast Cancer Cell Properties

30 clones overexpressing CASD1 have been isolated and assessed for OAcGD2 expression. Two clones were selected according to their level of OAcGD2/CASD1 overexpression. These clones exhibited higher migrative and invasive capacities with no modification of their proliferation rates, suggesting a role of OAcGD2 in breast cancer migration and invasion. Although O-acetylated gangliosides such as OAcGD3 and OAcGD2 are now considered as TACAs, there is very little data in the literature regarding their roles in cancer cell biology. OAcGD3 protects leukemic blasts, Jurkat cells and glioblastoma cells from apoptosis. Moreover, increased levels of 9-O-acetylated Neu5Ac corresponding notably to elevated 9-OAcGD3 were detected in acute lymphocytic leukemia (ALL) cells that developed resistance against vincristine or nilotinib, two drugs with different cytotoxic mechanisms. Treatment of ALL cells by a sialate acetyl esterase that cleaved the 9-O-acetyl residues from sialic acids made these cells more sensitive to both drugs. SIAE overexpression in hamster melanoma cells induced a loss of OAcGD3, altered cell morphology, a slower growth rate, and lower melanogenesis activity compared to controls. Previous studies suggest a role of OAcGD2 in cancer cell properties, for example an anti-OAcGD2 mAb c.8B6 monoclonal antibody inhibited glioblastoma and neuroblastoma cell proliferation in vitro and in vivo. The inventors described here higher migrative and invasive capacities of SUM159PT clones overexpressing CASD1 and 9-OAcGD2, with no modification in their proliferation rates. Importantly, CASD1 overexpression could modulate the expression of other O-acetylated gangliosides or sialylated glycosphingolipids (globo, lacto/neolacto series), which could also modify the biological properties of cancer cells.

CASD1 is ubiquitously expressed in all tissues and cells according to the Human Protein Atlas. In agreement, all breast cancer cell lines tested in this study express CASD1 at variable levels.

For now, CASD1 is mentioned only in very few publications in Pubmed (NCBI), showing the limited knowledge available regarding the physiological role of CASD1. The difficulties encountered for cloning and isolation of SOAT render the deciphering of O-acetylated ganglioside biosynthesis mechanisms complicated. The inventors' data indicate a role of CASD1 in GD2 O-acetylation in breast cancer cells and a CASD1-dependent pathway for both 9-OAcGD2 and 9-0AcGD3 in SUM159PT breast cancer cells and in CHO cells. In addition, increased tumorigenic properties of breast cancer cells over-expressing CASD1 and OAcGD2 were observed.

Altogether, the inventors' data allow to identify new markers and therapeutic targets for cancer treatment.

### Example 2: Biomarkers of OAcGD2 Expression

### Materials and Methods

### Cell Culture

Cell culture reagents were purchased from Lonza (Verviers, Belgium). Cells were routinely grown in monolayer culture and maintained at 37° C. in an atmosphere of 5% CO2. The human breast cancer cell line MDA-MB-231 GD3S+cells were grown in Dulbecco's modified Eagle's medium (DMEM) supplemented with 10% heat-inactivated fetal calf serum, 2 mmol/L L-glutamine and 1 mM sodium pyruvate.

### SiRNA Reverse Transfection

2.5 μL/well of 500 nM siGenome siRNA (Dharmacon/Horizon) was printed into blackwalled 384 well cell carrier ultra plates (Perkin Elmer) with Velocity 11 (Agilent) as described before (Chia et al., 2012, Mol. Syst. Biol., 8: 629.). Reverse siRNA transfection was performed by pre-mixing 0.1 μL of Dharmafect 1 transfection reagent (#T-2001-03, Horizon) with 7.4 μL of Optimem for 5 minutes. Addition of mixture to the siRNA plate was then performed with small multidrop combi cassette (Thermo-Fisher) and was left for complexation for 20 minutes with shaking. Addition of 2750 cells in 40 μL of DMEM supplemented with 10% FCS per well was then performed with multidrop combi standard cassette at medium speed (Thermo-Fisher). siRNA targeting-GD2S (L-011279-00-0020, Horizon), Polo like kinase 1 (PLK1; L-003290-00-0020, Horizon) and on-targeting pool (D-001810-10-20, Horizon) were used as screen controls.

### Immunofluorescence Staining and Automated Images Acquisition

After 72 h of incubation, transfected cells were fixed with 50 μL per well of 4% paraformaldehyde in 2% sucrose and 0.1M sodium phosphate buffer, 15 mM at 37° C. Cells were washed once with Hepes buffer 0.2M pH 7.4 and membrane permeabilization was performed in 5 μg/ml of digitonine in Hepes buffer for 20 min. Blocking was performed for 1 h with blocking buffer containing 0.2% gelatin, 2% BSA and 2% FCS. Aspiration of any liquid was performed with a 384 channels aspiration manifold at constant distance height from bottom of the well (V&P Scientific Inc). Sodium hydroxide treatment at 1 mM was added to selective control non-transfected well for the deacetylation of sialic acid. Staining of OAcGD2 was performed by incubation of 8B6 mAbs followed by suitable anti-mouse conjugated Alexa Fluor 488 secondary antibody at 1/500 dilution (Thermo Fisher Scientific). Each antibody was incubated successively for 1 h each on a 1 cm-span orbital shaker at 150 rpm. Nuclei were counterstained with 1 μg/ml Hoescht Thermo Fisher Scientific. Washings after antibody incubation were performed three times with 0.2M Hepes at pH 7.4 and 5 minutes shaking. All dispensing steps were performed with multidrop combi standard cassette. Stained plates were subjected to sequential channel acquisition for Hoechst/Alexa 488 with high content spinning disk confocal imager: phenix Opera (Perkin Elmer). Eight fields per well were acquired with 20X NA 1.0 water immersion objective with default laser power and exposure settings.

### Segmentation Pipeline for the Selection of the Hits

An OAcGD2 expression metric was derived with total cell thresholded fluorescence intensity obtained by immunodetection with 8B6 mAb in MDA-MB-231 GD3S+cells and was normalized with Hoeschst nuclei counts. Pools of four siRNAs per gene were arrayed in a series of 384-well plates. The segmentation pipeline applied for analysis of data derived with Columbus (Perkin Elmer) image analysis software and consisted of few module blocks: a basic flatfield correction for each image. Nuclei count detection with method B excluding nuclei object<50 μm² and with a 0.4 common intensity threshold. OAcGD2 signal detection with Image region-based algorithm with a threshold of 0.6 and with multiple objects detection, file hole algorithm on objects and exclusion of object size<2000 square pixel. The calculation of OAcGD2 fluorescent signal metric was then derived with the sum of pixel intensity for all objects over 2000 square pixels size divided by the nuclei number for all 8 fields image per well. The exclusion of objects with area less than 2000 square pixel size was applied to subfilter antibody artefacts.

### Results

### Key Controls

Key controls were designed to validate consistency of our workflow and to normalize plate to plate variations. In this screening, siRNA Non-Targeting pool (siNT) was added to empty wells of each 384-well plate as a negative control, siRNA targeting-Polo like kinase 1 (siPLK1) was used as siRNA transfection control. Finally, siRNA targeting GD2 synthase (siGD2S) was used as a modulator of OAcGD2 staining fluorescent signal. siPLK1 induced over 95% decrease in nuclei count as compared to NT transfected wells and confirmed efficient siRNA transfection in all plates tested. Nuclei count between siNT-transfected wells or non-transfected control wells were very similar highlighting the specific siPLK1 killing mediated effect and the very low level of transfection toxicity induced by our transfection reagents.

SiGD2S mediates a reduced intensity of OAcGD2 fluorescent signal in all plates tested when compared to signal in siNT control wells but showed some changes in silencing performance between the first screen replicate versus the second screen replicate. Due to this variability, the chemical treatment was used to control the modulation of OAcGD2 fluorescent signal. Sodium hydroxide has been shown previously to deacetylate all acetyl groups present on the cell surface and blocked efficiently antigen recognition by 8B6 mAb. Fixed cells in selected control wells were thus treated with NaOH 0.1M before primary antibody staining. OAcGD2 staining obtained on NaOH-treated wells was consistently abolished when compared to siNT transfected wells. The Z factor for siGD2S versus siNT was equal to 0.30 whereas the Z factor for NaOH treated wells versus siNT was around 0.70. Since Z factor readout with siNT and NaOH treated wells showed better consistency, these 2 key controls were used to calibrate screen data for normalization.

### Formatting Results

SiRNA that showed high toxicity in the wells (total nuclei counts<1000) were excluded from the analysis. The number of wells affected by toxicity constituted fewer than 5% of total siRNA tested. To minimize variations between plate data, each datapoint was normalized with the alternative score dependent on plate mean values of control siNT and plate mean values of NaOH treated controls wells (Moreau et al., 2011, Cell, 146:303-317) by applying the following formula:

$$Alternative score = \frac{Xi - {\overline{X}}_{siNT}}{{\overline{X}}_{siNT} - {\overline{X}}_{NaOH}}$$

The cutoff for the selection of OAcGD2 up or downregulating hits was defined with the first derivative approach (Moreau et al., 2011, Cell, 146:303-317). Genes were ranked according to their alternative score value from the minimum to the maximum. The cutoffs were designed before the largest spike at lowest ranks and highest ranks of the first derivative.

### Selection of Hits

Pearson correlation (r or R²) factor was calculated on the basis of alternative scores on both replicates datapoints. In this screening experiment, we obtained r=0.79 and R²=0.63 showing that the linear correlation between the two replicates was acceptable and that the screen outcome was reasonably reproducible. Five genes were identified as upregulating OAcGD2 expression (Table 1).

TABLE 1
Genes modulating OAcGD2 expression
in MDA-MB-231 GD3S+ cells

| | | Replicate 1 alt | Replicate 2 alt | OAcGD2 |
|---|---|---|---|---|
| Gene | NMID | score | score | modulation |
| CERK | NM_022766 | 5.24 | 6.19 | Upregulation |
| PIK3C2A | NM_002645 | 13.10 | 8.13 | Upregulation |
| PDK3 | NM_005391 | 5.84 | 2.23 | Upregulation |
| MERTK | NM_006343 | 4.99 | 2.44 | Upregulation |
| NME3 | NM_002513 | 5.52 | 5.46 | Upregulation |

### Discussion

The OAcGD2 siRNA screen was analyzed based on the fluorescence intensity obtained by immunodetection using 8B6 mAb in MDA-MB-231 GD3S+cells. Results were replicated and analyzed by combining first derivatives cutoff method and visual confirmation of hits on both replicates leading to the identification of 5 hits upregulating OAcGD2 expression. Results obtained could be interpreted based on the identification of hits but also on the images acquired. Images obtained from the transfection of the MDA-MB-231 GD3S+using siRNA targeting the different genes selected from our screen revealed significant variations of cellular morphology for the hits upregulating OAcGD2 with an intracellular and membrane staining pattern. Cells transfected with siRNA like siCERK and siPI3KC2A showed extended shape. Modifications of cell morphology after siRNA transfection can occur frequently depending on the depleted gene.

### Example 3: Transcriptomic Analyses

### Materials and Methods

### Transcriptomics Analyses

Data are TCGA datasets obtained from SurvExpress. Analyses were performed using SurvExpress optimized algorithm. Hazard ratio was calculated in patient populations computationally identified as high or low expression level of the gene of interest (i.e., CASD1, CERK, PIK3C2A, B4GALTN1, ST8SIA1) based on individual signature expression in TCGA datasets by SurvExpress optimized algorithm. The datasets analyzed were Sarcoma (SARC); Pheochromocytoma and Paraganglioma (PCPG); Uterine Corpus Endometrial Carcinoma (UCEC); Thyroid carcinoma (THCA); Thymoma (THYM); Testicular Germ Cell Tumors (TGCT); Stomach adenocarcinoma (STAD); Skin Cutaneous Melanoma (SKCM); Prostate adenocarcinoma (PRAD); Pancreatic adenocarcinoma (PAAD); Ovarian serous cystadenocarcinoma (OV); Lung squamous cell carcinoma (LUSC); Lung adenocarcinoma (LUAD); Liver hepatocellular carcinoma (LIHC); Kidney PAN cancer (KIPAN); Acute Myeloid Leukemia (LAML); Head and Neck squamous cell carcinoma (HNSC); Uveal Melanoma (UVM); Esophageal carcinoma (ESCA); Colon and Rectum adenocarcinoma (COADREAD); Cervical squamous cell carcinoma and endocervical adenocarcinoma (CESC); Breast invasive carcinoma (BRCA); Gliomas (GBM and LGG); Bladder Urothelial Carcinoma (BLCA); Cholangiocarcinoma (CHOL); Adrenocortical carcinoma (ACC).

### Results

The relationship between CASD1, B4GALNT1, ST8SIA1, CERK, PIK3C2A gene expression and patient overall survival was investigated in the TCGA datasets using the SurvExpress online tool. Hazard ratios represent the probability of patient death, where a hazard ratio of two means that a patient from the high expression group has twice the probability of dying compared to a patient from the low expression group. We analyzed the impact of CASD1, B4GALNT1, ST8SIA1, CERK, PIK3C2A gene expression, alone or in combination, on patient survival.

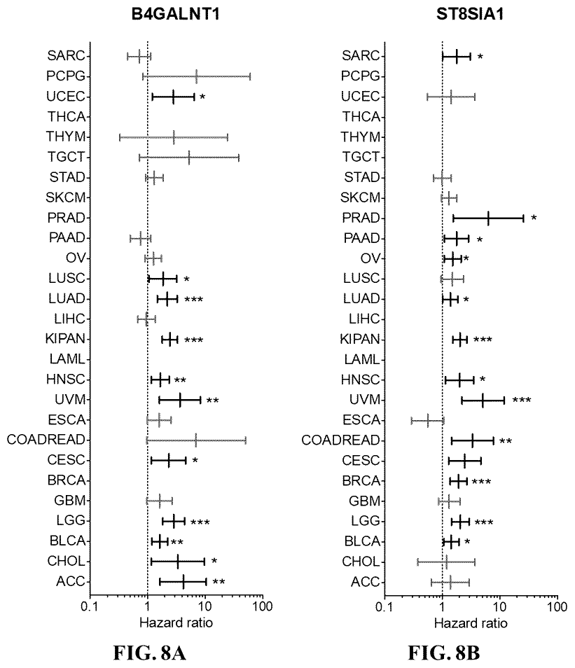

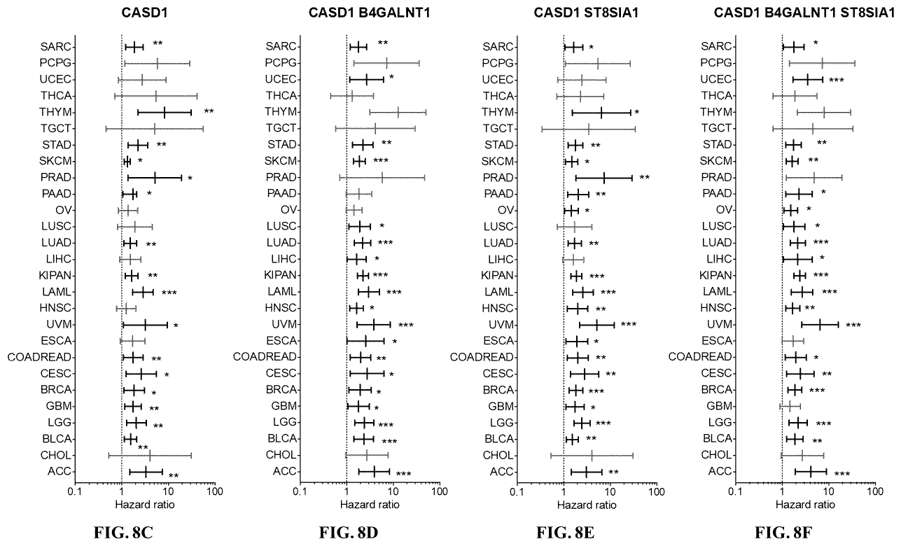

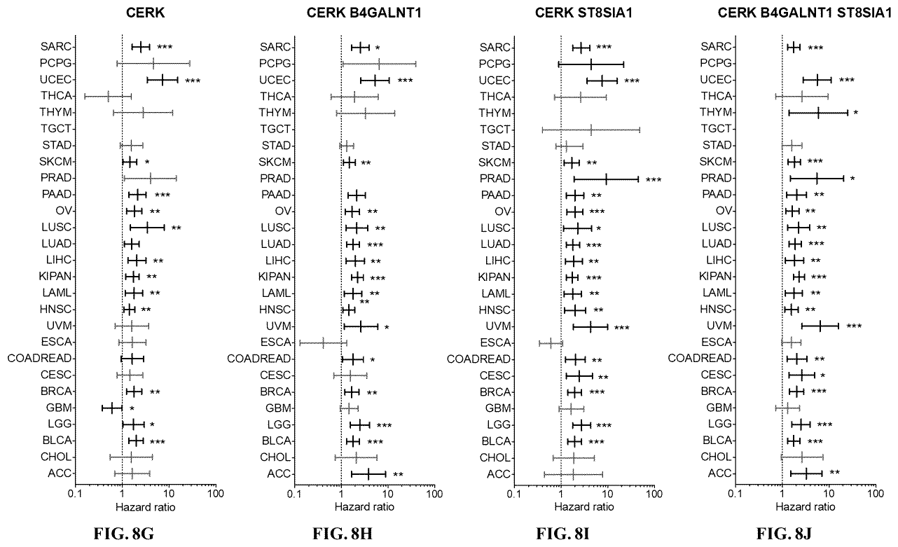

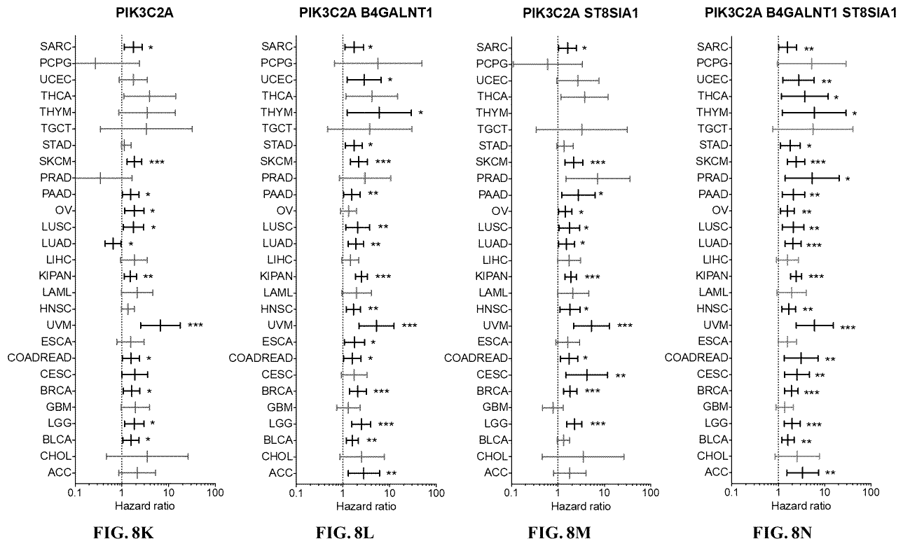

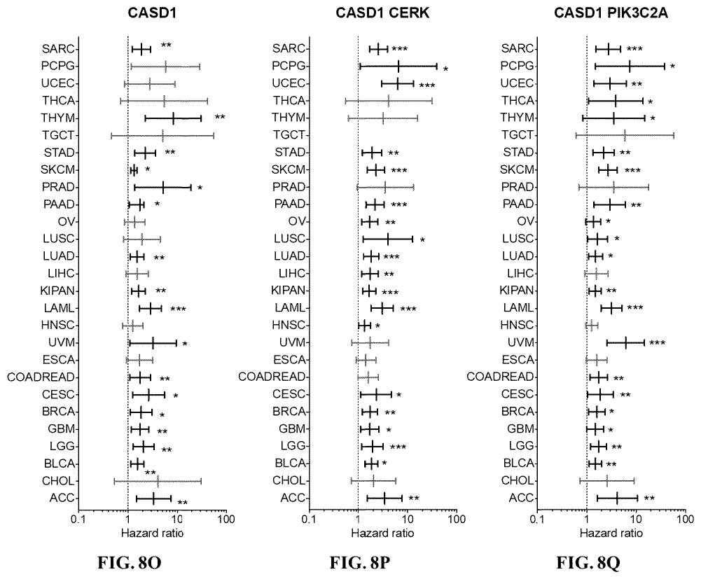

As shown on FIG. 8A, high B4GALNT1 gene expression was correlated with poor prognosis in 11 cancer types out of 22 including uterine corpus endometrial carcinoma (UCEC), Lung squamous cell carcinoma (LUSC), lung adenocarcinoma (LUAD), kidney cancer (KIPAN), head and neck squamous cell carcinoma (HNSC), Uveal Melanoma (UVM), Cervical squamous cell carcinoma and endocervical adenocarcinoma (CESC), gliomas (LGG), bladder urothelial carcinoma (BLCA), Cholangiocarcinoma (CHOL), and Adrenocortical carcinoma (ACC).

As shown on FIG. 8B, high ST8SIA1 gene expression was correlated with poor prognosis in 12 cancer types out of 21 which are Sarcoma (SARC), Prostate adenocarcinoma (PRAD), Pancreatic adenocarcinoma (PAAD), Ovarian serous cystadenocarcinoma (OV), Lung adenocarcinoma (LUAD), Kidney PAN cancer (KIPAN), Head and Neck squamous cell carcinoma (HNSC), Uveal melanoma (UVM), Colon and Rectum adenocarcinoma (COADREAD), Breast invasive carcinoma (BRCA), gliomas (LGG) and Bladder Urothelial Carcinoma (BLCA).

As shown on FIG. 8C, high CASD1 gene expression was associated with poorer survival in 17 cancer types out of 27 including sarcoma (SARC), lung adenocarcinoma (LUAD), colon and rectum adenocarcinoma (COADREAD), breast invasive carcinoma (BRCA), and glioblastoma (GBM and LGG). We analyzed CASD1 gene expression in combination with one or two glycosyltransferases encoding genes: B4GALNT1 and ST8SIA1.

Most cancer types kept a poor prognostic (14 of 17) when CASD1 and B4GALNT1 genes were both highly expressed (see FIG. 8D). Moreover, this combination induced an increase of the hazard ratio in 5 other cancer types, in which high gene expression of both CASD1 and B4GALNT1 was associated with poorer survival: uterine corpus endometrial carcinoma (UCEC), liver hepatocellular carcinoma (LIHC), lung squamous cell carcinoma (LUSC), head and neck squamous cell carcinoma (HNSC), and esophageal carcinoma (ESCA).

As shown on FIG. 8E, combination of high expression of both CASD1 and ST8SIA1 genes was associated with poorer outcome than high CASD1 gene expression alone in the same cancer types. High CASD1 and ST8SIA1 gene expression increased the hazard ratio in ovarian serous cystadenocarcinoma (OV), head and neck squamous cell carcinoma (HNSC) and esophageal carcinoma (ESCA).

High expression of the three CASD1, B4GALNT1 and ST8SIA genes was associated with poorer prognosis in 19 out of 27 TCGA datasets (see FIG. 8F).

As shown on FIG. 8G, high CERK gene expression was associated with poorer survival in 13 cancer types of 26 including sarcoma (SARC), uterine corpus endometrial carcinoma (UCEC), Skin Cutaneous Melanoma (SKCM), Pancreatic adenocarcinoma (PAAD), Ovarian serous cystadenocarcinoma (OV), Lung squamous cell carcinoma (LUSC), Liver hepatocellular carcinoma (LIHC), Kidney PAN cancer (KIPAN), Acute Myeloid Leukemia (LAML), Head and Neck squamous cell carcinoma (HNSC), Breast invasive carcinoma (BRCA), gliomas (LGG), and Bladder Urothelial Carcinoma (BLCA).

As shown on FIG. 8H, high expression of both CERK and B4GALNT1 genes worsened the prognostic of 4 cancer types: Lung adenocarcinoma (LUAD), uveal melanoma (UVM), Colon and Rectum adenocarcinoma (COADREAD), and adrenocortical carcinoma (ACC).

As shown on FIG. 8I, high expression of both CERK and ST8SIA1 genes was associated with poorer outcome in 18 cancer types. High CERK/ST8SIA1 gene expression increased the hazard ratio of prostate adenocarcinoma (PRAD), Lung adenocarcinoma (LUAD), uveal melanoma (UVM), Colon and Rectum adenocarcinoma (COADREAD) and endocervical adenocarcinoma (CESC). High expression of the 3 CERK/B4GALNT1/ST8SIA genes was associated with poorer prognosis in 20 out of 25 TCGA datasets (see FIG. 8J).

As shown on FIG. 8K, high PIK3C2A gene expression was associated with poorer survival in 11 cancer types out of 27.

High expression of PIK3C2A gene in combination with B4GALNT1 (FIG. 8L) worsened the prognostic of 6 cancer types: uterine corpus endometrial carcinoma (UCEC), thymoma (THYM), stomach adenocarcinoma (STAD), Lung adenocarcinoma (LUAD), head and neck squamous cell carcinoma (HNSC), esophageal carcinoma (ESCA) and adrenocortical carcinoma (ACC).

High expression of both PIK3C2A and ST8SIA1 genes was associated with poorer outcome in 13 cancer types (FIG. 8M). High PIK3C2A/ST8SIA1 gene expression increased the hazard ratio of lung adenocarcinoma (LUAD), head and neck squamous cell carcinoma (HNSC) and cervical squamous cell carcinoma and endocervical adenocarcinoma (CESC).

High expression of the 3 genes PIK3C2A/B4GALNT1/S T8SIA was associated with poorer prognosis in 20 out of 27 TCGA datasets (FIG. 8N).

High CASD1 gene expression was correlated with poor survival in 17 TCGA datasets (FIG. 8C and FIG. 8O), high CERK gene expression in 13 TCGA datasets (FIG. 8G) and high PIK3C2A gene expression in 11 TCGA datasets (FIG. 8K).

As shown on FIG. 8P, high expression of both CASD1 and CERK genes was associated with poorer prognosis in 19 out of 26 TCGA datasets. High expression of both CASD1 and CERK worsened the prognostic of 6 cancer types, as compared to high expression of CASD1 alone: Pheochromocytoma and Paraganglioma (PCPG), Uterine Corpus Endometrial Carcinoma (UCEC), Ovarian serous cystadenocarcinoma (OV), Lung squamous cell carcinoma (LUSC), Liver hepatocellular carcinoma (LIHC), and Head and Neck squamous cell carcinoma (HNSC).

As shown on FIG. 8Q, high expression of both CASD1 and PIK3C2A genes was associated with poor prognosis in 21 out of 27 TCGA datasets. High expression of both CASD1 and PIK3C2A worsened the prognosis of 5 cancer types, as compared to high expression of CASD1 alone: Pheochromocytoma and Paraganglioma (PCPG), Uterine Corpus Endometrial Carcinoma (UCEC), Thyroid carcinoma (THCA), Ovarian serous cystadenocarcinoma (OV), and Lung squamous cell carcinoma (LUSC).

In conclusion, our data show that high expression level of CASD1 correlated with poor survival in many cancer types, and may thus be used as a prognostic biomarker in these cancers.

Moreover, a combined high expression level of 2 or 3 genes correlated with poorer survival in more cancer types than the individual genes, showing that the use of combined biomarkers is relevant as a prognostic tool.

### Example 4: Effect of CERK Inhibition on GD2 O-Acetylation and Migratory Properties in Breast Cancer Cells Line MDA-MB231 GD3S+

### Materials and Methods

### Cell Culture

MDA-MB231 GD3S+cells were obtained as described in Cazet et al. (Biol Chem. 2009; 390(7):601-609). Cells were routinely grown in monolayer culture and maintained at 37° C. in an atmosphere of 5% CO₂. Cells were grown in Dulbecco's modified Eagle's medium (DMEM, Lonza) supplemented with 10% heat-inactivated fetal calf serum, 2 mmol/L L-Glutamine.

siRNA Transfection

Transfections were performed with 10 μM of siRNA control non-targeting (D001810-10-20, Horizon), siRNA targeting CASD1 (L-016926-01-0010, Horizon), or two different siRNA targeting CERK: CERK2 (D-004061-02-0010, Horizon), or CERK4 (D-004061-02-0010, Horizon) and 4 μL of RNAimax (#137781, Thermo fischer Scientific) in 500 μL of UltraMEM (Lonza). In 6-well plates, 150,000 cells were grown in 1,5 mL of DMEM and transfection mix. Cells were collected 72 hours after transfection for qPCR or immunocytochemistry experiments.

RNA Extraction, cDNA Synthesis and qPCR

Total RNA was extracted from cells using the Nucleospin RNA II kit (Macherey-Nagel, Germany). The amount of extracted RNA was quantified using a DeNovix DS-11 spectrophotometer (DeNovix Inc., USA) and the purity of the RNA was checked by the ratio of the absorbance at 260 and 280 nm. Total RNA was subjected to reverse transcription using the Maxima First Strand cDNA Synthesis Kit (ThermoFisher Scientific, Villeneuve d'Ascq, France) according to the protocol provided by the manufacturer. The oligonucleotide sequences (Eurogentec, Seraing, Belgium) used as primers for the PCR reactions are given in SEQ ID NO: 31 and SEQ ID NO: 32. qPCR and subsequent data analysis were performed using the AriaMx Quantitative System (Stratagene, La Jolla, CA, USA). PCR reaction (25 μL) contained 12.5 μL of the 2X Luna Master Mix (NEB), 300 nM of primers and 4 μL of cDNA (1:40). DNA amplification was performed with the following thermal cycling profile: initial denaturation at 94° C. for 10 min, 40 cycles of amplification (denaturation at 94° C. for 20 s, annealing at Tm for 20 s, and extension at 60° C. for 30 s). Hypoxanthine-guanine PhosphoRibosylTransferase (HPRT) gene was used to normalize the expression of genes of interest (oligonucleotides sequences used as PCR primers reactions are given in SEQ ID NO: 54 and SEQ ID NO: 55). The fluorescence monitoring occurred at the end of each cycle. The analysis of amplification was performed using the AriaMx 6.0 software. For each primer pair, the specificity of the amplification was checked by recording the dissociation curves. All experiments were performed in triplicate. The quantification was performed by the method described by Pfaffl (Nucleic Acids Research, 2001; 29(9):e45).

### CERK Inhibitor Treatment

Cells were seeded in 6-well plates (150,000 cells/well) with coverslips in 2 mL of DMEM medium. After 24 hours, the medium was replaced with DMEM containing 1 μM of the CERK inhibitor NVP231 (SigmaN9289). After 2 h, 4 h, 6 h, 8 h, 20 h, 24 h or 48 h of CERK inhibitor treatment, cells were collected for immunocytochemistry experiments.

### Immunocytochemistry and Confocal Microscopy

Transfected cells were grown on glass coverslips and were fixed for 20 min in 4% paraformaldehyde. Cells were washed three times with PBS 1X and membrane permeabilization was performed in 5 μg/mL digitonine in PBS 1X for 20 min. After 3 washes, cells were saturated in PBS 1X-BSA 0.5% blocking buffer. Coverslips were transferred in humid chamber and cells were incubated 2 hours with anti-OAcGD2 monoclonal antibody 8B6 mouse IgG3 (OGD2 Pharma, Nantes, France) at 20 μg/mL. Cells were washed three times with PBS 1X-BSA 0.5% and incubated 1 hour with secondary antibody Alexa Fluor 488 donkey anti-mouse IgG at 3 μg/mL (Invitrogen). After 3 washes, cells were incubated with 1 μg/mL of DAPI (Sigma, #D9542) for 7 min. Coverslips were mounted in fluorescent mounting medium (Dako). Coverslips were observed under the A1 Nikon confocal microscope with a 60X oil immersion objective. The green fluorescence was acquired with λex=488 nm and λem=500-530 nm, DAPI with λex=350 nm and λem=460 nm. Images were processed with ImageJ and backgrounds generated by secondary antibody alone were deducted. Mean fluorescence intensity was calculated with macro using ImageJ.

### Transwell Assays

Cells were seeded in 12-well plates containing migration chamber in serum-free medium. Below the chamber, medium with serum was added into the wells in the presence or absence of 1 μM of NVP231. After 24 hours incubation at 37° C., wells were fixed with 4% paraformaldehyde and non-migratory cells were swapped with cotton swabs. Cells were washed three times and nuclei were stained with DAPI. Membranes were cut out and mounted between glass slide and coverslip with fluorescent mounting medium. Nuclei were counted using A1 Nikon confocal microscopy.

### Results

In order to study the effect of CERK inhibition on O-acetylation of the GD2 ganglioside expression and on the migration capacity of the breast cancer cell line MDA-MB231 GD3S+, two strategies were used: CERK inhibition using a CERK inhibitory and using siRNA directed against CERK mRNA.

### Effect of a CERK Inhibitor

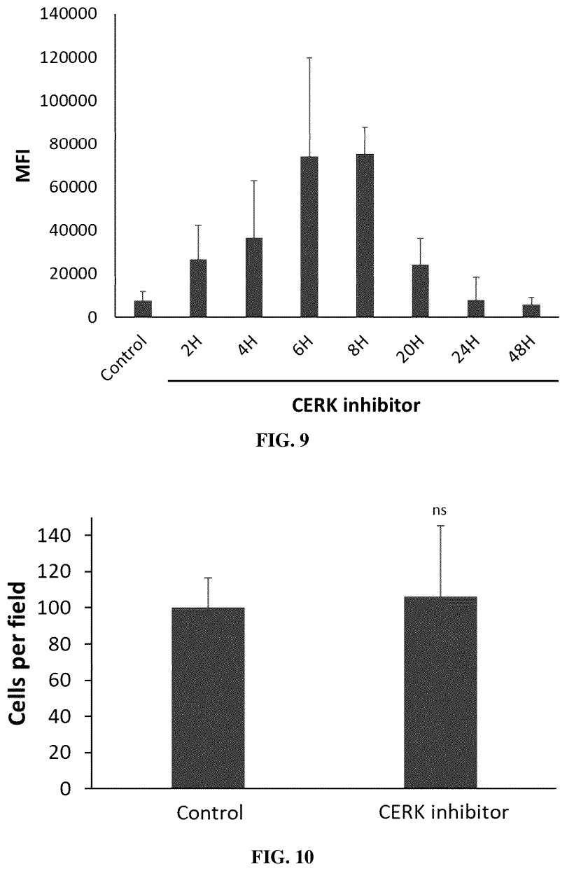

Breast cancer cells MDA-MB-231 GD3S+, overexpressing the GD3 synthase, were treated with a CERK inhibitor for 2, 4, 6, 8, 20, 24 or 48 hours. At the end of the culture, cells were stained by immunocytochemistry using an antibody specifically recognizing the O-acetylated GD2 ganglioside, and the mean fluorescence intensity was measured by confocal microscopy. As shown on FIG. 9, CERK inhibition induced a transitory overexpression of OAcGD2, with a gradual increase of OAcGD2 expression from 2 to 8 hours, followed by a gradual decrease after 8 hours of treatment.

The effect of CERK inhibition on the migration capacity of the cells was also evaluated. Breast cancer cells MDA-MB-231 GD3S+were cultured in plates containing migration chamber in the presence or absence of the CERK inhibitor. After 24 hours of culture, cells were counted to evaluate migration. As shown on FIG. 10, treatment with the CERK inhibitor had no effect on the migration capacity of the cells. The number of cells counted in the bottom chamber of the Transwell plate was similar between cells treated with the CERK inhibitor or untreated.

These data demonstrate that CERK inhibition with the CERK inhibitor increased OAcGD2 expression in MDA-MB-231 GD3S+cells, but did not increase the migration capacity of the cells.

Effect of a siRNA Targeting CERK

Breast cancer cells MDA-MB-231 GD3S+were transiently transfected using either a control siRNA (siControl), a siRNA directed against CASD1 mRNA (siCASD1) or a siRNA directed against CERK mRNA (siCERK2 or siCERK4).

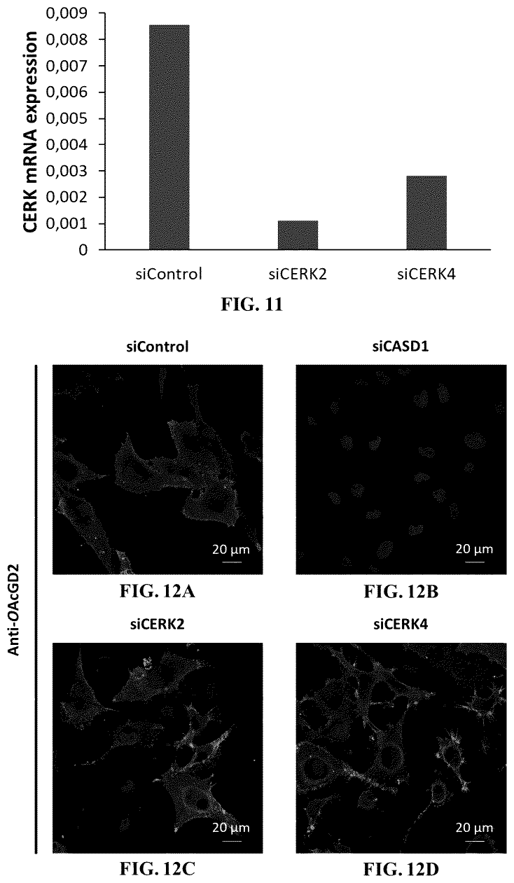

First, the efficiency of the siRNA directed against CERK mRNA (siCERK2 and siCERK4) was evaluated by quantification of CERK mRNA expression by qPCR. As shown on FIG. 11, both CERK siRNA, siCERK2 and siCERK4, were able to decrease the expression level of CERK mRNA as compared to the control siRNA (siControl).

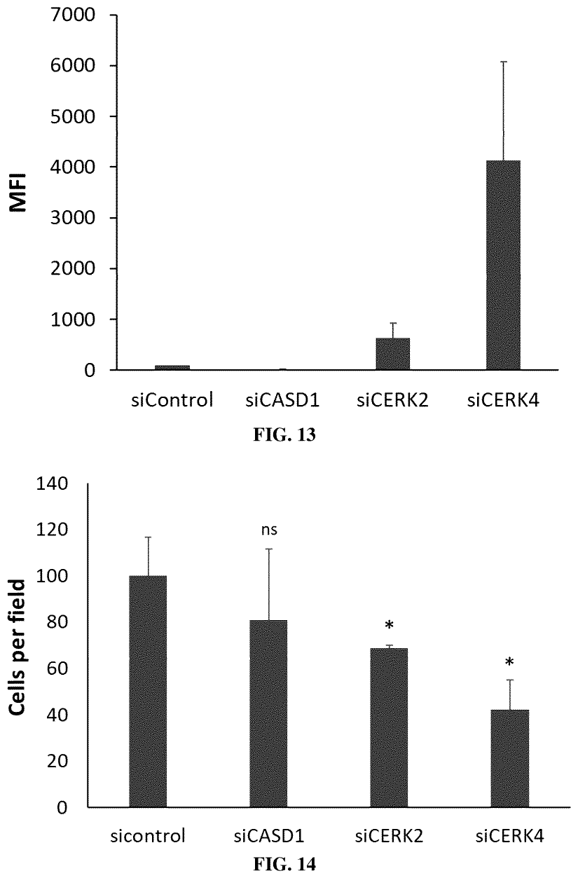

Then, the expression of the O-acetylated GD2 ganglioside was measured on the cells by immunocytochemistry using an antibody specifically recognizing the O-acetylated GD2 ganglioside. Cells were analyzed by confocal microscopy. FIGS. 12A-D show representative confocal microscopy photographs of cells transfected with the following siRNA: control siRNA (FIG. 12A), siRNA CASD1 (FIG. 12B), siRNA CERK2 (FIG. 12C) and siRNA CERK4 (FIG. 12D). Mean fluorescence intensity was also measured for each condition and is plotted on FIG. 13. As shown on the images and by quantification, inhibition of CASD1 mRNA using a siRNA directed against CASD1 mRNA (FIGS. 12B and 13) decreased OAcGD2 expression, as compared to the control siRNA (siControl, FIGS. 12A and 13). On the other hand, inhibition of CERK mRNA using a siRNA directed against CERK mRNA (siCERK2, FIGS. 12C and 13, and siCERK4, FIGS. 12D and 13) increased the expression of the O-acetylated GD2 ganglioside, as compared to the control siRNA (siControl, FIGS. 12A and 13).

Additionally, the migration capacity of the cells transiently transfected with the siRNA was evaluated. As shown on FIG. 14, both CERK siRNA (siCERK2 and siCERK4) induced a significant decrease of the cell migration capacity, as compared to the control siRNA (siControl), while CASD1 siRNA (siCASD1) did not significantly impact cell migration.

These data demonstrate that CERK inhibition using CERK siRNA induced an increase of the O-acetylated GD2 ganglioside expression in breast cancer cell line MDA-MB231 GD3S+, but decreased the migratory capacity of these cells.

1-14. (canceled)

15. An in vitro method of selecting a subject suffering from a cancer expressing the O-acetylated-GD2 ganglioside for treatment targeting said cancer, said method comprising the steps of:
a1) measuring CASD1 expression level as a biomarker in a biological sample of the subject;
a1′) optionally, measuring the expression level of at least one biomarker selected from the group consisting of GD2S, GD3S, OAcGD2, GD2, CERK, PIK3C2A, PDK3, MERTK and NME3, in a biological sample of the subject; and
b) based on the level measured at step a1), and optionally at step a1′), selecting said subject to undergo treatment targeting said cancer expressing the O-acetylated-GD2 ganglioside.

16. An in vitro method of monitoring the response of a subject suffering from a cancer expressing the O-acetylated-GD2 ganglioside to a treatment targeting said cancer, said method comprising the steps of:
a1) measuring CASD1 expression level as a biomarker in a biological sample of the subject;
a1′) optionally, measuring the expression level of at least one biomarker selected from the group consisting of GD2S, GD3S, OAcGD2, GD2, CERK, PIK3C2A, PDK3, MERTK and NME3, in a biological sample of the subject; and
b) based on the level measured at step a1), and optionally at step a1′), monitoring the response of said subject to said treatment.

17. An in vitro method of diagnosing a cancer expressing the O-acetylated-GD2 ganglioside in a subject, said method comprising the steps of:
a1) measuring CASD1 expression level as a biomarker in a biological sample of the subject;
a1′) optionally, measuring the expression level of at least one biomarker selected from the group consisting of GD2S, GD3S, OAcGD2, GD2, CERK, PIK3C2A, PDK3, MERTK and NME3, in a biological sample of the subject; and
b) based on the level measured at step a1), and optionally at step a1′), diagnosing a cancer expressing the O-acetylated-GD2 ganglioside in said subject.

18. The in vitro method according to claim 15, further comprising a step a2) of comparing the expression level measured at step a1), and/or optionally at step a1′), with a threshold value.

19. The in vitro method according to claim 18, wherein the subject is selected to undergo a treatment targeting said cancer expressing the O-acetylated-GD2 ganglioside, or is diagnosed as suffering from a cancer expressing the O-acetylated-GD2 ganglioside, if the expression level measured at step a1), and/or optionally at step a1′), is higher than the threshold value.

20. The in vitro method according to claim 15, wherein the biological sample is selected from the group consisting of a blood sample, a serum sample, a plasma sample, a urine sample, a tissue sample from a biopsy and a cell sample from a biopsy.

21. The in vitro method according to claim 15, wherein the expression level measured at step a1), and/or optionally at step a1′), is measured at the DNA or RNA level, preferably by RT-PCR, RT-qPCR, Northern Blot, hybridization techniques, microarrays or sequencing.

22. The in vitro method according to claim 15, wherein the expression level measured at step a1), and/or optionally at step a1′), is measured at the protein level, preferably by FACS, immunohistochemistry, mass spectrometry, western blot associated with cell fractionation, enzyme-linked immunosorbent assay (ELISA), sandwich ELISA, fluorescent-linked immunosorbent assay (FLISA), enzyme immunoassay (EIA), radioimmunoassay (RIA) or image analysis.

23. The in vitro method according to claim 15, wherein said treatment comprises an antibody that binds to the O-acetylated-GD2 ganglioside.

24. The in vitro method according to claim 15, wherein said cancer expressing the O-acetylated-GD2 ganglioside is characterized by the presence of cells expressing the O-acetylated-GD2 ganglioside at their cell surface in the subject.

25. The in vitro method according to claim 15, wherein said cancer expressing the O-acetylated-GD2 ganglioside is selected from the group consisting of neuroblastoma, glioma (including glioblastoma), retinoblastoma, Ewing's family of tumors, sarcoma (including rhabdomyosarcoma, osteosarcoma, leiomyosarcoma, liposarcoma, and fibrosarcoma), lung cancer (including small cell lung cancer), breast cancer, melanoma (including uveal melanoma), metastatic renal carcinoma, head and neck cancer, hematological cancers (including leukemia, Hodgkin lymphoma, non-Hodgkin lymphoma and myeloma), colorectal cancer, pancreatic cancer, prostate cancer, liver cancer, bladder cancer, gastric/stomach cancer, cervical cancer, endometrial cancer, neuroendocrine cancer, esophageal cancer, ovarian cancer, skin cancer, kidney cancer, soft tissue sarcoma, adrenal cancer, testicular cancer, thymic cancer (including thymoma) and thyroid cancer.

26. A method for treating a cancer expressing the O-acetylated-GD2 ganglioside in a subject, comprising administering to a subject in need thereof a therapeutical effective amount of an inhibitor of CASD1.

27. A method for treating a cancer expressing the O-acetylated-GD2 ganglioside in a subject, comprising administering to a subject in need thereof a therapeutical effective amount of an inhibitor of a biomarker selected from the group consisting of CERK, PIK3C2A, PDK3, MERTK, NME3 and EZH2, in combination with a therapy targeting the O-acetylated-GD2 ganglioside.


SEQUENCE LISTING (55 sequences)

SEQ ID NO: 1 - protein, 797 aa, Homo sapiens
  CASD1 human protein
MAALAYNLGKREINHYFSVRSAKVLALVAVLLLAACHLASRRYRGNDSCEYLLSSGRFLGEKVWQPHSCMMHKYKISEAKNCLVDKHIAFIGDSRIRQLFYSFVKIINPQFKEEGNKHENIPFEDKTASVKVDFLWHPEVNGSMKQCIKVWTEDSIAKPHVIVAGAATWSIKIHNGSSEALSQYKMNITSIAPLLEKLAKTSDVYWVLQDPVYEDLLSENRKMITNEKIDAYNEAAVSILNSSTRNSKSNVKMFSVSKLIAQETIMESLDGLHLPESSRETTAMILMNVYCNKILKPVDGSCCQPRPPVTLIQKLAACFFTLSIIGYLIFYIIHRNAHRKNKPCTDLESGEEKKNIINTPVSSLEILLQSFCKLGLIMAYFYMCDRANLFMKENKFYTHSSFFIPIIYILVLGVFYNENTKETKVLNREQTDEWKGWMQLVILIYHISGASTFLPVYMHIRVLVAAYLFQTGYGHFSYFWIKGDFGIYRVCQVLFRLNFLVVVLCIVMDRPYQFYYFVPLVTVWFMVIYVTLALWPQIIQKKANGNCFWHFGLLLKLGFLLLFICFLAYSQGAFEKIFSLWPLSKCFELKGNVYEWWFRWRLDRYVVFHGMLFAFIYLALQKRQILSEGKGEPLFSNKISNFLLFISVVSFLTYSIWASSCKNKAECNELHPSVSVVQILAFILIRNIPGYARSVYSSFFAWFGKISLELFICQYHIWLAADTRGILVLIPGNPMLNIIVSTFIFVCVAHEISQITNDLAQIIIPKDNSSLLKRLACIAAFFCGLLILSSIQDKSKH

SEQ ID NO: 2 - protein, 533 aa, Homo sapiens
  GD2 synthase human protein
MWLGRRALCALVLLLACASLGLLYASTRDAPGLRLPLAPWAPPQSPRRPELPDLAPEPRYAHIPVRIKEQVVGLLAWNNCSCESSGGGLPLPFQKQVRAIDLTKAFDPAELRAASATREQEFQAFLSRSQSPADQLLIAPANSPLQYPLQGVEVQPLRSILVPGLSLQAASGQEVYQVNLTASLGTWDVAGEVTGVTLTGEGQADLTLVSPGLDQLNRQLQLVTYSSRSYQTNTADTVRFSTEGHEAAFTIRIRHPPNPRLYPPGSLPQGAQYNISALVTIATKTFLRYDRLRALITSIRRFYPTVTVVIADDSDKPERVSGPYVEHYLMPFGKGWFAGRNLAVSQVTTKYVLWVDDDFVFTARTRLERLVDVLERTPLDLVGGAVREISGFATTYRQLLSVEPGAPGLGNCLRQRRGFHHELVGFPGCVVTDGVVNFFLARTDKVREVGFDPRLSRVAHLEFFLDGLGSLRVGSCSDVVVDHASKLKLPWTSRDAGAETYARYRYPGSLDESQMAKHRLLFFKHRLQCMTSQ

SEQ ID NO: 3 - protein, 356 aa, Homo sapiens
  GD3 synthase human protein
MSPCGRARRQTSRGAMAVLAWKFPRTRLPMGASALCVVVLCWLYIFPVYRLPNEKEIVQGVLQQGTAWRRNQTAARAFRKQMEDCCDPAHLFAMTKMNSPMGKSMWYDGEFLYSFTIDNSTYSLFPQATPFQLPLKKCAVVGNGGILKKSGCGRQIDEANFVMRCNLPPLSSEYTKDVGSKSQLVTANPSIIRQRFQNLLWSRKTFVDNMKIYNHSYIYMPAFSMKTGTEPSLRVYYTLSDVGANQTVLFANPNFLRSIGKFWKSRGIHAKRLSTGLFLVSAALGLCEEVAIYGFWPFSVNMHEQPISHHYYDNVLPFSGFHAMPEEFLQLWYLHKIGALRMQLDPCEDTSLQPTS

SEQ ID NO: 4 - protein, 537 aa, Homo sapiens
  CERK human protein
MGATGAAEPLQSVLWVKQQRCAVSLEPARALLRWWRSPGPGAGAPGADACSVPVSEIIAVEETDVHGKHQGSGKWQKMEKPYAFTVHCVKRARRHRWKWAQVTFWCPEEQLCHLWLQTLREMLEKLTSRPKHLLVFINPFGGKGQGKRIYERKVAPLFTLASITTDIIVTEHANQAKETLYEINIDKYDGIVCVGGDGMFSEVLHGLIGRTQRSAGVDQNHPRAVLVPSSLRIGIIPAGSTDCVCYSTVGTSDAETSALHIVVGDSLAMDVSSVHHNSTLLRYSVSLLGYGFYGDIIKDSEKKRWLGLARYDFSGLKTFLSHHCYEGTVSFLPAQHTVGSPRDRKPCRAGCFVCRQSKQQLEEEQKKALYGLEAAEDVEEWQVVCGKFLAINATNMSCACRRSPRGLSPAAHLGDGSSDLILIRKCSRFNFLRFLIRHTNQQDQFDFTFVEVYRVKKFQFTSKHMEDEDSDLKEGGKKRFGHICSSHPSCCCTVSNSSWNCDGEVLHSPAIEVRVHCQLVRLFARGIEENPKPDSHS

SEQ ID NO: 5 - protein, 1686 aa, Homo sapiens
  PIK3C2A human protein
MAQISSNSGFKECPSSHPEPTRAKDVDKEEALQMEAEALAKLQKDRQVTDNQRGFELSSSTRKKAQVYNKQDYDLMVFPESDSQKRALDIDVEKLTQAELEKLLLDDSFETKKTPVLPVTPILSPSFSAQLYFRPTIQRGQWPPGLPGPSTYALPSIYPSTYSKQAAFQNGFNPRMPTFPSTEPIYLSLPGQSPYFSYPLTPATPFHPQGSLPIYRPVVSTDMAKLFDKIASTSEFLKNGKARTDLEITDSKVSNLQVSPKSEDISKFDWLDLDPLSKPKVDNVEVLDHEEEKNVSSLLAKDPWDAVLLEERSTANCHLERKVNGKSLSVATVTRSQSLNIRTTQLAKAQGHISQKDPNGTSSLPTGSSLLQEVEVQNEEMAAFCRSITKLKTKFPYTNHRTNPGYLLSPVTAQRNICGENASVKVSIDIEGFQLPVTFTCDVSSTVEIIIMQALCWVHDDLNQVDVGSYVLKVCGQEEVLQNNHCLGSHEHIQNCRKWDTEIRLQLLTFSAMCQNLARTAEDDETPVDLNKHLYQIEKPCKEAMTRHPVEELLDSYHNQVELALQIENQHRAVDQVIKAVRKICSALDGVETLAITESVKKLKRAVNLPRSKTADVTSLFGGEDTSRSSTRGSLNPENPVQVSINQLTAAIYDLLRLHANSGRSPTDCAQSSKSVKEAWTTTEQLQFTIFAAHGISSNWVSNYEKYYLICSLSHNGKDLFKPIQSKKVGTYKNFFYLIKWDELIIFPIQISQLPLESVLHLTLFGILNQSSGSSPDSNKQRKGPEALGKVSLPLFDFKRFLTCGTKLLYLWTSSHTNSVPGTVTKKGYVMERIVLQVDFPSPAFDIIYTTPQVDRSIIQQHNLETLENDIKGKLLDILHKDSSLGLSKEDKAFLWEKRYYCFKHPNCLPKILASAPNWKWVNLAKTYSLLHQWPALYPLIALELLDSKFADQEVRSLAVTWIEAISDDELTDLLPQFVQALKYEIYLNSSLVQFLLSRALGNIQIAHNLYWLLKDALHDVQFSTRYEHVLGALLSVGGKRLREELLKQTKLVQLLGGVAEKVRQASGSARQVVLQRSMERVQSFFQKNKCRLPLKPSLVAKELNIKSCSFFSSNAVPLKVTMVNADPMGEEINVMFKVGEDLRQDMLALQMIKIMDKIWLKEGLDLRMVIFKCLSTGRDRGMVELVPASDTLRKIQVEYGVTGSFKDKPLAEWLRKYNPSEEEYEKASENFIYSCAGCCVATYVLGICDRHNDNIMLRSTGHMFHIDFGKFLGHAQMFGSFKRDRAPFVLTSDMAYVINGGEKPTIRFQLFVDLCCQAYNLIRKQTNLFLNLLSLMIPSGLPELTSIQDLKYVRDALQPQTTDAEATIFFTRLIESSLGSIATKFNFFIHNLAQLRFSGLPSNDEPILSFSPKTYSFRQDGRIKEVSVFTYHKKYNPDKHYIYVVRILREGQIEPSFVFRTFDEFQELHNKLSIIFPLWKLPGFPNRMVLGRTHIKDVAAKRKIELNSYLQSLMNASTDVAECDLVCTFFHPLLRDEKAEGIARSADAGSFSPTPGQIGGAVKLSISYRNGTLFIMVMHIKDLVTEDGADPNPYVKTYLLPDNHKTSKRKTKISRKTRNPTFNEMLVYSGYSKETLRQRELQLSVLSAESLRENFFLGGVTLPLKDFNLSKETVKWYQLTAATYL

SEQ ID NO: 6 - protein, 406 aa, Homo sapiens
  PDK3 human protein
MRLFRWLLKQPVPKQIERYSRFSPSPLSIKQFLDFGRDNACEKTSYMFLRKELPVRLANTMREVNLLPDNLLNRPSVGLVQSWYMQSFLELLEYENKSPEDPQVLDNFLQVLIKVRNRHNDVVPTMAQGVIEYKEKFGFDPFISTNIQYFLDRFYTNRISFRMLINQHTLLFGGDTNPVHPKHIGSIDPTCNVADVVKDAYETAKMLCEQYYLVAPELEVEEFNAKAPDKPIQVVYVPSHLFHMLFELFKNSMRATVELYEDRKEGYPAVKTLVTLGKEDLSIKISDLGGGVPLRKIDRLFNYMYSTAPRPSLEPTRAAPLAGFGYGLPISRLYARYFQGDLKLYSMEGVGTDAVIYLKALSSESFERLPVFNKSAWRHYKTTPEADDWSNPSSEPRDASKYKAKQ

SEQ ID NO: 7 - protein, 999 aa, Homo sapiens
  MERTK human protein
MGPAPLPLLLGLFLPALWRRAITEAREEAKPYPLFPGPFPGSLQTDHTPLLSLPHASGYQPALMFSPTQPGRPHTGNVAIPQVTSVESKPLPPLAFKHTVGHIILSEHKGVKFNCSISVPNIYQDTTISWWKDGKELLGAHHAITQFYPDDEVTAIIASFSITSVQRSDNGSYICKMKINNEEIVSDPIYIEVQGLPHFTKQPESMNVTRNTAFNLTCQAVGPPEPVNIFWVQNSSRVNEQPEKSPSVLTVPGLTEMAVFSCEAHNDKGLTVSKGVQINIKAIPSPPTEVSIRNSTAHSILISWVPGFDGYSPFRNCSIQVKEADPLSNGSVMIFNTSALPHLYQIKQLQALANYSIGVSCMNEIGWSAVSPWILASTTEGAPSVAPLNVTVFLNESSDNVDIRWMKPPTKQQDGELVGYRISHVWQSAGISKELLEEVGQNGSRARISVQVHNATCTVRIAAVTRGGVGPFSDPVKIFIPAHGWVDYAPSSTPAPGNADPVLIIFGCFCGFILIGLILYISLAIRKRVQETKFGNAFTEEDSELVVNYIAKKSFCRRAIELTLHSLGVSEELQNKLEDVVIDRNLLILGKILGEGEFGSVMEGNLKQEDGTSLKVAVKTMKLDNSSQREIEEFLSEAACMKDFSHPNVIRLLGVCIEMSSQGIPKPMVILPFMKYGDLHTYLLYSRLETGPKHIPLQTLLKFMVDIALGMEYLSNRNFLHRDLAARNCMLRDDMTVCVADFGLSKKIYSGDYYRQGRIAKMPVKWIAIESLADRVYTSKSDVWAFGVTMWEIATRGMTPYPGVQNHEMYDYLLHGHRLKQPEDCLDELYEIMYSCWRTDPLDRPTFSVLRLQLEKLLESLPDVRNQADVIYVNTQLLESSEGLAQGSTLAPLDLNIDPDSIIASCTPRAAISVVTAEVHDSKPHEGRYILNGGSEEWEDLTSAPSAAVTAEKNSVLPGERLVRNGVSWSHSSMLPLGSSLPDELLFADDSSEGSEVLM

SEQ ID NO: 8 - protein, 169 aa, Homo sapiens
  NME3 human protein
MICLVLTIFANLFPAACTGAHERTFLAVKPDGVQRRLVGEIVRRFERKGFKLVALKLVQASEELLREHYAELRERPFYGRLVKYMASGPVVAMVWQGLDVVRTSRALIGATNPADAPPGTIRGDFCIEVGKNLIHGSDSVESARREIALWFRADELLCWEDSAGHWLYE

SEQ ID NO: 9 - DNA, 32 bp, Artificial Sequence
  CASD1 PCR primer
gctcgggatccgcggctctggcctacaacctg

SEQ ID NO: 10 - DNA, 37 bp, Artificial Sequence
  CASD1 PCR primer
gctcgctcgagatgttttgatttatcttgaatggatg

SEQ ID NO: 11 - DNA, 54 bp, Artificial Sequence
  V5 epitope pre-hybridized oligonucleotide
agcttcgaatgggtaagcctatccctaaccctctcctcggtctcgattctacgg

SEQ ID NO: 12 - DNA, 54 bp, Artificial Sequence
  V5 epitope pre-hybridized oligonucleotide
gatcccgtagaatcgagaccgaggagagggttagggataggcttacccattcga

SEQ ID NO: 13 - DNA, 42 bp, Artificial Sequence
  Myc epitope pre-hybridized oligonucleotide
tcgaggaacaaaaactcatctcagaagaggatctgaattaat

SEQ ID NO: 14 - DNA, 42 bp, Artificial Sequence
  Myc epitope pre-hybridized oligonucleotide
ctagattaattcagatcctcttctgagatgagtttttgttcc

SEQ ID NO: 15 - DNA, 33 bp, Artificial Sequence
  S94A mutagenesis primer
gcatttattggagatgccagaattcgtcaattg

SEQ ID NO: 16 - DNA, 33 bp, Artificial Sequence
  S94A mutagenesis primer
caattgacgaattctggcatctccaataaatgc

SEQ ID NO: 17 - DNA, 80 bp, Artificial Sequence
  ST8SIA1 PCR primer (G3DS)
gctaagcttcgaatgggtaagcctatccctaaccctctcctcggtctcgattctacgggtaccagcccctgcgggcgggc

SEQ ID NO: 18 - DNA, 31 bp, Artificial Sequence
  ST8SIA1 PCR primer (G3DS)
gctgcggccgcctaggaagtgggctggagtg

SEQ ID NO: 19 - protein, 20 aa, Artificial Sequence
  self-cleaving 2A peptide of equine rhinitis A virus
QCTNYALLKLAGDVESNPGP

SEQ ID NO: 20 - DNA, 25 bp, Artificial Sequence
  GD3S/self-cleaving 2A peptide/GD2S PCR primer
atagcggccgcatgagcccctgcgg

SEQ ID NO: 21 - DNA, 29 bp, Artificial Sequence
  GD3S/self-cleaving 2A peptide/GD2S PCR primer
gctctctagatcactcggcggtcatgcac

SEQ ID NO: 22 - DNA, 23 bp, Artificial Sequence
  exon 2-specific target sequence
ttgcatttatcggagattccagg

SEQ ID NO: 23 - DNA, 18 bp, Artificial Sequence
  Target region PCR primer
gctgtgcctaacagtttg

SEQ ID NO: 24 - DNA, 19 bp, Artificial Sequence
  Target region PCR primer
tggcaagtttttccatgag

SEQ ID NO: 25 - DNA, 24 bp, Artificial Sequence
  Target region PCR primer
tgaagcaaagaattgccttgtaga

SEQ ID NO: 26 - DNA, 26 bp, Artificial Sequence
  Target region PCR primer
cttatttccttcttctttaaactggg

SEQ ID NO: 27 - DNA, 18 bp, Artificial Sequence
  CASD1 PCR primer
atgttcacaacgccacgg

SEQ ID NO: 28 - DNA, 18 bp, Artificial Sequence
  CASD1 PCR primer
caggaaccatccacaggc

SEQ ID NO: 29 - DNA, 33 bp, Artificial Sequence
  NeuD PCR primer
cgccgcggatccgaaaaaataaccttaaaatgc

SEQ ID NO: 30 - DNA, 44 bp, Artificial Sequence
  NeuD PCR primer
gtccgctcgagttaaaatagattaaaaattttttttgattttag

SEQ ID NO: 31 - DNA, 20 bp, Artificial Sequence
  CASD1 PCR primer
gtggattttctgtggcatcc

SEQ ID NO: 32 - DNA, 20 bp, Artificial Sequence
  CASD1 PCR primer
aagcgcttcactgctaccat

SEQ ID NO: 33 - DNA, 21 bp, Artificial Sequence
  B4GALNT1 PCR primer (G2DS)
cagcgctctagtcacgattgc

SEQ ID NO: 34 - DNA, 20 bp, Artificial Sequence
  B4GALNT1 PCR primer (G2DS)
ccacggtaaccgttgggtag

SEQ ID NO: 35 - DNA, 19 bp, Artificial Sequence
  ST8SIA1 PCR primer (G3DS)
gcgatgcaatctccctcct

SEQ ID NO: 36 - DNA, 20 bp, Artificial Sequence
  ST8SIA1 PCR primer
ttcccgaattatgctgggat

SEQ ID NO: 37 - protein, 12 aa, Artificial Sequence
  8B6 mAb LC-CDR1
QSLLKNNGNTFL

SEQ ID NO: 38 - protein, 9 aa, Artificial Sequence
  8B6 mAb LC-CDR3
SQSTHIPYT

SEQ ID NO: 39 - protein, 8 aa, Artificial Sequence
  8B6 mAb HC-CDR1
EFTFTDYY

SEQ ID NO: 40 - protein, 10 aa, Artificial Sequence
  8B6 mAb HC-CDR2
IRNRANGYTT

SEQ ID NO: 41 - protein, 10 aa, Artificial Sequence
  8B6 mAb HC-CDR3
ARVSNWAFDY

SEQ ID NO: 42 - protein, 112 aa, Artificial Sequence
  SITE 1: X=D or E
  Humanized anti-OAcGD2 VL consensus sequence
  SITE 2: X=V or I
  SITE 9: X=L or A
  SITE 10: X=S or T
  SITE 12: X=P or S
  SITE 13: X=V or L or A
  SITE 14: X=S or T
  SITE 15: X=L or P or V
  SITE 17: X=D or Q or E
  SITE 18: X=Q or P or R
  SITE 19: X=A or V
  SITE 20: X=S or T
  SITE 21: X=I or L
  SITE 22: X=S or T
  SITE 25: X=S or A
  SITE 29: X=L or V
  SITE 30: X=L or V
  SITE 32: X=N or S
  SITE 33: X=N or Q
  SITE 34: X=G or A or S
  SITE 35: X=N or Y or S
  SITE 36: X=T or N or S
  SITE 37: X=F or Y
  SITE 39: X=H or N or S or A or Y or G
  SITE 41: X=Y or F
  SITE 42: X=L or Q
  SITE 44: X=K or R
  SITE 47: X=Q or K
  SITE 48: X=S or A or V
  SITE 50: X=K or Q or R
  SITE 51: X=L or R or V
  SITE 55: X=K or G or L
  SITE 56: X=V or A or G
  SITE 58: X=N or T
  SITE 60: X=L or D or A
  SITE 61: X=S or T
  SITE 63: X=V or I
  SITE 65: X=D or A or S
  SITE 75: X=Y or D
  SITE 79: X=K or T
  SITE 81: X=S or N
  SITE 82: X=R or S
  SITE 83: X=V or L
  SITE 84: X=E or Q
  SITE 85: X=A or P
  SITE 88: X=L or V or F
  SITE 89: X=G or A
  SITE 90: X=V or T
  SITE 92: X=F or Y
  SITE 94: X=S or M or Q
  SITE 96: X=S or A
  SITE 97: X=T or Y
  SITE 98: X=H or Q or N
  SITE 99: X=I or T or Q
  SITE 100: X=P or S
  SITE 105: X=G or Q
XXVMTQSPXXLXXXXGXXXXXXCRXSQSXXKXXXXXXLXWXXQXPGXXPXXLIYXXSXRXXGXPXRFSGSGSGTXFTLXIXXXXXEDXXXYXCXQXXXXXYTFGXGTKVEIK

SEQ ID NO: 43 - protein, 119 aa, Artificial Sequence
  SITE 1: X=E or Q
  Humanized anti-OAcGD2 VH consensus sequence
  SITE 5: X=V or L
  SITE 13: X=Q or K
  SITE 16: X=G or R
  SITE 23: X=A or T
  SITE 24: X=T or A
  SITE 26: X=E or G
  SITE 30: X=T or S or G
  SITE 32: X=Y or H
  SITE 35: X=T or H or N or S
  SITE 37: X=V or I
  SITE 48: X= L or V
  SITE 49: X= G or S
  SITE 50: X= F or Y
  SITE 51: X= I or T
  SITE 54: X= R or K or S
  SITE 55: X= A or S
  SITE 56: X=N or S
  SITE 57: X= G or A or S
  SITE 58: X= Y or G
  SITE 59: X= T or I
  SITE 60: X= T or I
  SITE 61: X= E or Y
  SITE 63: X= N or A
  SITE 64: X= P or A or D
  SITE 76: X= N or G
  SITE 77: X= S or A
  SITE 79: X= S or N
  SITE 80: X= I or S or T
  SITE 81: X= L or T or A
  SITE 89: X= R or K or Q
  SITE 90: X= T or A
  SITE 95: X= V or I or L
  SITE 99: X= A or T
  SITE 105: X=A or Y
  SITE 114: X= T or L
  SITE 115: X= L or V
XVQLXESGGGLVXPGXSLRLSCXXSXFTFXDXYMXWXRQAPGKGLEWXXXXRNXXXXXXXXYXXSVKGRFTISRDXXKXXXYLQMNSLXXEDTAXYYCXRVSNWXFDYWGQGTXXTVSS

SEQ ID NO: 44 - protein, 119 aa, Artificial Sequence
  Anti-OAcGD2 VH49A
EVQLVESGGGLVQPGRSLRLSCTTSEFTFTDYYMTWVRQAPGKGLEWLGFIRNRANGYTTEYNPSVKGRFTISRDNSKSILYLQMNSLKTEDTAVYYCARVSNWAFDYWGQGTLVTVSS

SEQ ID NO: 45 - protein, 119 aa, Artificial Sequence
  Anti-OAcGD2 VH72A
EVQLVESGGGLVQPGGSLRLSCATSEFTFTDYYMTWVRQAPGKGLEWLGFIRNRANGYTTEYNPSVKGRFTISRDNSKNSLYLQMNSLKTEDTAVYYCARVSNWAFDYWGQGTLVTVSS

SEQ ID NO: 46 - protein, 119 aa, Artificial Sequence
  Anti-OAcGD2 VH49BHS
EVQLVESGGGLVQPGRSLRLSCTTSEFTFTDYYMTWVRQAPGKGLEWLGFIRNKANGYTTEYNPSVKGRFTISRDNSKSILYLQMNSLKTEDTAVYYCARVSNWAFDYWGQGTLVTVSS

SEQ ID NO: 47 - protein, 119 aa, Artificial Sequence
  Anti-OAcGD2 VH72BHNPS
EVQLVESGGGLVQPGGSLRLSCATSEFTFSDYYMTWVRQAPGKGLEWLGFIRNKANGYTTEYNPSVKGRFTISRDNSKNSLYLQMNSLKTEDTAVYYCARVSNWAFDYWGQGTLVTVSS

SEQ ID NO: 48 - protein, 112 aa, Artificial Sequence
  Anti-OAcGD2 VL30A
DVVMTQSPLSLPVTLGQPASISCRSSQSLLKNNGNTFLHWYQQRPGQSPRLLIYKVSNRLSGVPDRFSGSGSGTDFTLKISRVEAEDVGVYFCSQSTHIPYTFGGGTKVEIK

SEQ ID NO: 49 - protein, 112 aa, Artificial Sequence
  Anti-OAcGD2 VL 28A
DVVMTQSPLSLPVTPGEPASISCRSSQSLLKNNGNTFLHWYLQKPGQSPQLLIYKVSNRLSGVPDRFSGSGSGTDFTLKISRVEAEDVGVYFCSQSTHIPYTFGQGTKVEIK

SEQ ID NO: 50 - protein, 112 aa, Artificial Sequence
  Anti-OAcGD2 VL 28Bs01/A2
DVVMTQSPLSLPVTPGEPASISCRSSQSLLKSNANTFLHWYLQKPGQSPQLLIYKVSNRLSGVPDRFSGSGSGTDFTLKISRVEAEDVGVYYCMQSTHIPYTFGQGTKVEIK

SEQ ID NO: 51 - protein, 112 aa, Artificial Sequence
  Humanized anti-OAcGD2 VL consensus sequence
  SITE 2: X=V or I
  SITE 14: X=S or T
  SITE 15: X=L or P
  SITE 17: X=D or Q or E
  SITE 18: X=Q or P
  SITE 30: X=L or V
  SITE 32: X=N or S
  SITE 34: X= G or A
  SITE 37: X=F or Y
  SITE 41: X=Y or F
  SITE 42: X=L or Q
  SITE 44: X=K or R
  SITE 50: X=K or Q or R
  SITE 60: X=L or A
  SITE 75: X=Y or D
  SITE 88: X=L or V
  SITE 92: X=F or Y
  SITE 94: X=S or M
  SITE 105: X=G or Q
DXVMTQSPLSLPVXXGXXASISCRSSQSLXKXNXNTXLHWXXQXPGQSPXLLIYKVSNRXSGVPDRFSGSGSGTXFTLKISRVEAEDXGVYXCXQSTHIPYTFGXGTKVEIK

SEQ ID NO: 52 - protein, 119 aa, Artificial Sequence
  Humanized anti-OAcGD2 VH consensus sequence
  SITE 16: X=G or R
  SITE 23: X=A or T
  SITE 24: X=T or A
  SITE 26: X=E or G
  SITE 30: X=T or S or G
  SITE 32: X=Y or H
  SITE 35: X=T or S
  SITE 54: X= R or K
  SITE 56: X=N or S
  SITE 57: X= G or S or A
  SITE 58: X= Y or G
  SITE 60: X= T or I
  SITE 61: X= E or Y
  SITE 63: X= N or A
  SITE 64: X= P or A
  SITE 79: X= S or N
  SITE 80: X= I or S
  SITE 81: X= L or A
  SITE 89: X= R or K
  SITE 99: X= A or T
  SITE 114: X= T or L
  SITE 115: X= L or V
EVQLVESGGGLVQPGXSLRLSCXXSXFTFXDXYMXWVRQAPGKGLEWLGFIRNXAXXXTXXYXXSVKGRFTISRDNSKXXXYLQMNSLXTEDTAVYYCXRVSNWAFDYWGQGTXXTVSS

SEQ ID NO: 53 - protein, 751 aa, Homo sapiens
  EZH2 human protein
MGQTGKKSEKGPVCWRKRVKSEYMRLRQLKRFRRADEVKSMFSSNRQKILERTEILNQEWKQRRIQPVHILTSVSSLRGTRECSVTSDLDFPTQVIPLKTLNAVASVPIMYSWSPLQQNFMVEDETVLHNIPYMGDEVLDQDGTFIEELIKNYDGKVHGDRECGFINDEIFVELVNALGQYNDDDDDDDGDDPEEREEKQKDLEDHRDDKESRPPRKFPSDKIFEAISSMFPDKGTAEELKEKYKELTEQQLPGALPPECTPNIDGPNAKSVQREQSLHSFHTLFCRRCFKYDCFLHRKCNYSFHATPNTYKRKNTETALDNKPCGPQCYQHLEGAKEFAAALTAERIKTPPKRPGGRRRGRLPNNSSRPSTPTINVLESKDTDSDREAGTETGGENNDKEEEEKKDETSSSSEANSRCQTPIKMKPNIEPPENVEWSGAEASMFRVLIGTYYDNFCAIARLIGTKTCRQVYEFRVKESSIIAPAPAEDVDTPPRKKKRKHRLWAAHCRKIQLKKDGSSNHVYNYQPCDHPRQPCDSSCPCVIAQNFCEKFCQCSSECQNRFPGCRCKAQCNTKQCPCYLAVRECDPDLCLTCGAADHWDSKNVSCKNCSIQRGSKKHLLLAPSDVAGWGIFIKDPVQKNEFISEYCGEIISQDEADRRGKVYDKYMCSFLFNLNNDFVVDATRKGNKIRFANHSVNPNCYAKVMMVNGDHRIGIFAKRAIQTGEELFFDYRYSQADALKYVGIEREMEIP

SEQ ID NO: 54 - DNA, 20 bp, Artificial Sequence
  HPRT PCR primer (forward)
gccagactttgttggatttg

SEQ ID NO: 55 - DNA, 25 bp, Artificial Sequence
  HPRT PCR primer (reverse)
ctctcatcttaggctttgtattttg